(19) United States
(12) Patent Application Publication
(10) Pub. No.: US 2025/0230436 A1
(43) Pub. Date: Jul. 17, 2025

(54) RNA CIRCULARIZATION

(71) Applicant: SUZHOU ABOGEN BIOSCIENCES CO., LTD, Suzhou, Jiangsu (CN)

(72) Inventors: Shaojun QI, Suzhou, Jiangsu (CN); Peng GAO, Suzhou, Jiangsu (CN); Bo YING, Suzhou, Jiangsu (CN)

(21) Appl. No.: 18/853,398

(22) PCT Filed: Dec. 29, 2023
(86) PCT No.: PCT/CN2023/143083

§ 371 Date: Oct. 1, 2024

(30) Foreign Application Priority Data
Dec. 29, 2022 (WO) PCT/CN2022/143232
Mar. 31, 2023 (WO) PCT/CN2023/085331
Sep. 1, 2023 (WO) PCT/CN2023/116485

(51) Int. Cl. C12N 15/113; C12N 15/67

(52) U.S. Cl. CPC C12N 15/113; C12N 15/67; C12N 2310/124; C12N 2310/532

(57) ABSTRACT

The disclosure relates to novel RNA ribozyme constructs encoding foreign proteins or functional RNAs, with a circularization system based on group I introns, which are capable of self-circularizing with high efficiency without introducing extraneous fragments, as well as to methods of using the constructs to make circular RNAs.

### CROSS REFERENCE TO RELATED APPLICATIONS

The present application claims priority to International Application Nos. PCT/CN2022/143232 filed on Dec. 29, 2022, PCT/CN2023/085331 filed on Mar. 31, 2023 and PCT/CN2023/116485 filed on Sep. 1, 2023. The contents of the above-referenced applications are hereby incorporated by reference in their entireties.

### FIELD

The disclosure relates to novel RNA constructs encoding foreign proteins or functional RNAs, with a circularization system based on group I introns, which are capable of self-circularizing with high efficiency without introducing extraneous fragments, as well as to methods of using the constructs to make circular RNAs.

### BACKGROUND

Messenger RNA (mRNA) is a type of single-stranded RNA involved in protein synthesis. In vitro transcribed (IVT) mRNAs have recently attracted much attention as novel agents with great therapeutic potential. Especially, the successful use of mRNA vaccines for COVID-19 has proven the safety and efficacy of mRNA therapeutic agents in vivo. Because of its short development cycle, flexibility in design, and strong immune activation, mRNA vaccines have been rapidly validated for their safety and efficacy in combating infectious diseases such as Covid-19. However, the use of mRNA in nonvaccine therapies such as protein replacement is limited by several factors including mRNA stability, poor persistence of expression in vivo, immunogenicity, and limited range of expressing cell types. Linear single-stranded mRNA requires adding a 5′ cap and 3′ polyA tail or even incorporating modified nucleotides like 1 my to guarantee stability and expression levels in vivo while reducing the risk of unwanted immunogenicity. Moreover, even with these modifications, mRNA is susceptible to exonuclease digestion, resulting in a short half-life both in vitro and in vivo.

Circular RNA (circRNA) is a type of single-stranded RNA which forms a 3′-5′ covalently closed loop. CircRNAs are created by a non-canonical splicing process termed “backsplicing”, whereby the spliceosome fuses a splice donor site in a downstream exon (5′ splice site) to a splice acceptor site in an upstream exon (3′ splice site). Unlike linear mRNAs, circRNAs do not require a 5′-cap or 3′-poly (A) tail for their stability. The closed ring structure of circRNAs protects them from exonuclease-mediated degradation, rendering them resistant to several mechanisms of RNA turnover and having a 2.5-fold longer half-life compared to their linear mRNA counterparts. Moreover, circRNAs have beneficial features not shared by mRNAs, such as reduced immunogenicity and extended translation duration. For these reasons, circRNAs have been explored as therapeutic agents.

CircRNAs are generally noncoding, as they lack the 5′-cap structure, but several studies have provided evidence that some circRNAs can be translated into proteins. Engineered circRNA with cap-independent translation elements such as internal ribosome entry sites (IRES) or N⁶-methyladenosine (m6A) modifications can also facilitate protein translation in vivo. Like mRNAs, circRNAs can also be delivered via lipid nanoparticles (LNPs) to provide in vivo expression, which may be more sustained than linear mRNAs.

CircRNAs can be generated post-transcriptionally in living cells by plasmids carrying minigene sequences. Since spliceosome-mediated backsplicing is a major mechanism of circularization in vivo, most circRNA minigenes have at least exonic regions containing the sequence to be circularized, as well as 5′ and 3′ flanking intronic sequences containing splicing motifs. However, this vector transcription-dependent circularization can still produce variable amounts of unwanted heterologous by-products that cannot be easily identified or purified in vivo. In addition, this approach requires plasmid vectors to be efficiently delivered into the nucleus, making technical development difficult, while double-stranded DNAs also carry the risk of integrating into the genome.

Protein ligase and ribozyme assays are commonly used for in vitro preparation of circRNAs. Enzyme ligation-mediated circularization usually requires a complementary splint (a DNA or RNA oligo) to bring both ends of the RNA molecule closer and then catalysis by several enzymes from bacteriophage T4, including T4 DNA ligase, T4 RNA ligase 1, and T4 RNA ligase 2. However, all these ligase-mediated circularizations are relatively inefficient, especially for large RNA molecules. In addition, the generation of intermolecular end-joining by-products in the ligation reaction cannot be avoided entirely, leading to complicated system optimization and unfavorable production-scale-up.

Ribozyme-mediated RNA circularizations can also be performed by the permuted intron and exon (PIE) method based on the group I intron or group II intron self-splicing system.

The group I introns are naturally occurring cis-splicing ribozymes that can splice an RNA transcript and remove themselves from the primary transcript by autocatalyzing two consecutive trans-esterification reactions and joining the two flanking exons (see FIG. 58). Native group I introns do not require assistance from the spliceosome or other proteins to self-splice but rely on magnesium and free guanosine nucleotides to initiate and complete the reaction. This process leads to ligation of the exons flanking the intron and circularization of the internal intron to generate an intronic circRNA.

Helices P1 to P9 (and the intervening junctions and loops) assemble to form the catalytic core of group I introns. In general, helix P1 comprises at least 4-6 base pairs from the 5′ intron and 5′ exon, ending with a conserved G-U wobble base pair (5′-GNNNNN-3′ in intron or 5′-NNNNNU-3′ in exon), which contributes to 5′ splice site recognition. In addition, the P1 extension region (or “P1ex”) is important for the 5′ splicing reaction rate and splicing site recognition. The sequence ‘GNNNNN’ is also known as the internal guide sequence (IGS). For some group I introns, helix P10 is formed after the first step of splicing and involves base pairing between the 3′ intron and 3′ exon. The 3′ splice site is partially recognized through a conserved guanine at the 3′ end of the intron, termed Omega G (ωG). In some cases, the 3′ splice site accuracy can be improved by introducing or enhancing P9.0 or even P9.2 structures.

Previous studies provided a permuted intron-exon (PIE) splicing strategy using a modified group I intron, including placement of the 5′ half of the group I intron to the tail of the exon and transferring the remaining 3′ half to the head of the same exon. See, e.g., R. A. Wesselhoeft, P. S. Kowalski, D. G. Anderson, Engineering circular RNA for potent and stable translation in eukaryotic cells. Nat Commun 9, 2629 (2018); M. Puttaraju, M. D. Been, Group I permuted intron-exon (PIE) sequences self-splice to produce circular exons. Nucleic Acids Res 20, 5357-5364 (1992). This method achieves RNA circularization by a regular group I intron self-splicing reaction that includes two transesterifications at defined splice sites. Attack of the 5′ splice site by free GTP leads to the release of the 3′ end sequence (5′ half intron) of the PIE construct (first transesterification). The free 3′-OH group of the newly generated 3′ half exon attacks the 3′ splice site in the second transesterification reaction. This leads to the release of circRNA and 3′ half intron. Compared with enzymatic ligation, the PIE method can be used to circularize larger linear RNA precursors, it does not require additional protein ligase, and the reaction conditions and purification methods are easier to develop and optimize.

Circular RNAs encoding foreign proteins synthesized by the PIE method have been validated both in vitro and in vivo and retain the characteristics of low immunogenicity and longer translation duration, which broaden their applications. Based on these advantages, the PIE system is currently the most studied and widely used method for RNA circularization. Although the PIE system can achieve circularization of long fragments more efficiently than ligase-mediated methods, the splicing reaction introduces additional fragments (E1, E2, and spacer) from phage or Anabaena exons that may activate immune responses.

A PIE-group II intron system can achieve scarless circularization by optimizing exon binding sites (EBS) sequences to match the intron binding sites (IBS). However, the alteration of EBS greatly impacts splicing efficiency; complicated optimization and testing are required to guarantee efficient splicing, and the EBS-IBS pairs may in some cases be incompatible. The PIE system splits the ribozyme into two parts placed at the RNA construct's 3′ and 5′ terminals, which requires that the ribozyme fragments at both ends are correctly folded and spatially brought closer to form the complete ribozyme catalytic domain. However, the structure of the internal sequences may interfere with the ribozyme structure at both ends, which requires additional spacer sequences to separate the internal sequences and the ribozyme fragments at both ends.

There is a need for ribozyme-mediated circularization approaches that are simpler, faster, safer, more accurate, and more efficient than conventional processes.

### BRIEF SUMMARY

In an aspect, the disclosure provides novel RNA constructs (also referred to as “circular RNA precursors”) encoding foreign proteins or functional RNAs, with a circularization system based on group I introns, different from the PIE constructs, e.g., in having an intact ribozyme core. The RNA constructs are capable of self-circularizing with high efficiency without introducing extraneous fragments.

The novel RNA construct comprising,
a first recognizer sequence (R1) comprising a first pairing sequence;
a nucleotide sequence of interest (GOI) comprising a target site at its 3′ end;
a ribozyme core sequence operably linked to an internal guide sequence (IGS), wherein the ribozyme core sequence encodes a ribozyme core having the catalytic activity of a group I intron ribozyme; and
a second recognizer sequence (R2) comprising a second pairing sequence substantially complementary to the first pairing sequence;
wherein
the 5′ end nucleotide of the IGS and the 3′ end nucleotide of the target site form a non-Watson-Crick base pair to define a 5′ splice site;
R1 and R2 are positioned at opposite ends of the RNA construct, such that hybridization of the first and second pairing sequences results in the formation of a duplex-containing structure to define a 3′ splice site;
the GOI is positioned 5′ to the ribozyme core sequence and IGS; and
the RNA construct is capable of generating a circular RNA comprising the nucleotide sequence of interest through the catalytic activity of the ribozyme core.

This design retains the complete core domain of the ribozyme, which is more conducive to the correct folding of the ribozyme than the traditional PIE system. This method can also achieve circularization of the nucleotide sequence of interest without inclusion of exogenous sequence residues by mimicking the formation of a P1 duplex, selecting an arbitrary sequence (for example, ‘nnnnnu’ or ‘nnnnnc’) in a nucleotide sequence to be circularized as the target site sequence (simply guaranteeing that the target site sequence is unique in the RNA construct) and placing the sequence downstream the target site to the 3′ end of the nucleotide sequence to be circularized at the 5′ region of the GOI and the sequence from the 5′ end of the nucleotide sequence to be circularized to the target site at the 3′ region of the GOI, and then designing a corresponding IGS.

In some embodiments, the RNA construct comprises, from 5′ end to 3′ end,
R1 comprising a first pairing sequence and a 3′ end nucleotide ‘N’ (ωN);
GOI comprising a target site at its 3′ end,
IGS;
Ribozyme core sequence; and
R2 comprising a second pairing sequence;
wherein
ωN is any naturally occurring or modified nucleotide; and
the first pairing sequence and the second pairing sequence are substantially complementary to form a duplex-containing structure upstream of the ωN to define the 3′ splice site.

The disclosure further provides an RNA construct comprising, from 5′ end to 3′ end,
a first recognizer sequence (R1) comprising a nucleotide sequence ‘(Nₓ)ₛ(N_{y})ₜ(ωN)’ at its 3′ end;
a nucleotide sequence of interest (GOI) comprising a target site at its 3′ end;
an internal guide sequence (IGS);
a ribozyme core sequence encoding a ribozyme core which has the catalytic activity of a group I intron ribozyme; and a second recognizer sequence (R2) comprising a nucleotide sequence ‘(nₓ)_{w}’;
wherein
the 5′ end nucleotide of the IGS and the 3′ end nucleotide of the target site form a non-Watson-Crick base pair to define a 5′ splice site;
ωN, ‘Nₓ’, ‘nₓ’, and ‘N_{y}’ are each independently any naturally occurring or modified nucleotide;
t is an integer of 0-20;
s and w are each independently an integer of 1-200;
‘(Nₓ)ₛ’ and ‘(nₓ)_{w}’ are substantially complementary to form a duplex-containing structure upstream of the ωN to define a 3′ splice site; and
the RNA construct is capable of generating a circular RNA comprising the nucleotide sequence of interest through the catalytic activity of the ribozyme core.

In some embodiments, ωN is guanine (ωG).

In some embodiments, the ribozyme core sequence comprises a nucleotide sequence encoding the scaffold domain and catalytic domain of a group I intron; preferably, the ribozyme core sequence comprises or consists of the sequence from the IGS end to the sequence before the 5′ half of P9.0 duplex of a group I intron.

In some embodiments, the ribozyme core sequence is derived from a group IC1 (e.g., from Tetrahymena sp. (e.g., T. thermophile, T. cosmopolitanis, T. hyperangularis, T. malaccensis or T. pigmentosa) or Pneumocystis sp. (e.g., Pneumocystis carinii), IC2, IC3 (e.g., from Anabaena sp. PCC7120 or Azoarcus sp. BH72) or IA2 (e.g., from Bacteriophage Twort) intron.

The disclosure also provides an RNA construct comprising, from 5′ end to 3′ end,
a first nucleotide sequence comprising a sequence from a nucleotide ‘N_{q}’ to the 3′ end of a group I intron,
a nucleotide sequence of interest (GOI) comprising a target site at its 3′ end,
an internal guide sequence (IGS), and
a second nucleotide sequence comprising a sequence from the IGS end to a nucleotide ‘Nₚ’ of a group I intron;
wherein
the 5′ end nucleotide of the IGS and the 3′ end nucleotide of the target site form a non-Watson-Crick base pair to define a 5′ splice site;
‘Nₚ’ and ‘N_{q}’ are independently selected from any nucleotide from the 3′ end nucleotide of the 5′ half to the 5′ end nucleotide of the 3′ half of P9.0 duplex of the group I intron, and
‘Nₚ’ is located upstream of ‘N_{q}’ in the group I intron.

The disclosure also provides DNA constructs encoding the novel RNA constructs and methods of making circular RNAs using the novel constructs.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF DRAWINGS

FIG. 1 shows a schematic diagram of essential RNA sequence elements based on the cis-splicing circularization system.

FIG. 2 shows a schematic diagram of the circularization element with ribozyme P as an example according to some embodiments. The 5′ and 3′ recognizer sequences (Recognizer 1 and Recognizer 2) can simulate the formation of a P9.0 duplex mimic structure with at least two base pairs. Dashed boxes indicate unnecessary circularization elements.

FIG. 3 shows a schematic diagram of circularization elements with ribozyme T as an example according to some embodiments. R1 can pair with R2 to form a P9.0 duplex mimic, a P9.2 duplex mimic as well as a double-stranded region through base pairing between Homology Arm 1 (5′ homology arm) and Homology Arm 2 (3′ homology arm). Dashed boxes indicate unnecessary circularization elements.

FIG. 4A shows the GOI sequence structure when the target site ‘NNNNNU’ is located in the internal ribosome entry site (IRES) according to some embodiments. Dashed boxes indicate unnecessary circularization elements.

FIG. 4B shows the GOI sequence structure when the target site ‘NNNNNU’ is located in the open reading frame (ORF) according to some embodiments. Dashed boxes indicate unnecessary circularization elements.

FIG. 5 shows the sequence elements in the IGS region and the sequence elements in the GOI that form base pairs with the IGS according to some embodiments. Dashed boxes indicate unnecessary circularization elements.

FIG. 6A shows a schematic diagram of the circularization elements designed with ribozyme T according to some embodiments, where the backsplicing site is located in the ORF-GFP. Dashed boxes indicate unnecessary circularization elements.

FIG. 6B shows a schematic diagram of the circularization elements designed with ribozyme T according to some embodiments, where the backsplicing site is inside the IRES.

FIG. 7A shows a fragment analysis for the products of IVT of the circRNA precursor depicted in FIG. 6A with different Mg²⁺ concentrations. Circularized RNA (the peak on the left, represented by a circle) and remaining precursors (the peak on the right, represented by a curve) are indicated.

FIG. 7B shows the effect of Mg²⁺ concentrations on circularization rate (% CircRNA in total-FA data) in IVT system. The dotted line indicates the 40% circularization rate.

FIG. 7C shows the effect of Mg²⁺ concentrations on yield (total RNA) in IVT system. The dotted line indicates 200 μg yield.

FIG. 8A shows the migration mode of the purified products in the 2% E-Gel EX system for about 18 minutes. The product types of the corresponding bands are indicated on the right, respectively. RNase R digests almost all the linear RNAs but not circular RNAs, allowing circular RNAs to be enriched.

FIG. 8B shows the purity of the product with or without RNase R digestion detected by FA.

FIG. 9 shows the cell expression detection (FITC-GFP) of products prepared under different Mg²⁺ concentrations before and after treatment with RNase R.

FIG. 10 shows a schematic diagram of the circularization elements designed with ribozyme P as an example according to some embodiments, where the backsplicing site is located inside the IRES. Dashed boxes indicate unnecessary circularization elements.

FIG. 11 shows the migration mode of the purified products in the 2% E-Gel EX system for about 18 minutes. The product types of the corresponding bands are indicated on the right, respectively. RNase R digests almost all the linear RNAs but not circular RNAs, allowing circular RNAs to be enriched.

FIG. 12 shows the purity of the product with or without RNase R digestion detected by FA.

FIG. 13 shows a schematic diagram of the circularization elements designed based on ribozyme T with the IGS and target site split to the precursor's 5′ and 3′ regions, respectively, and the backsplicing site is located inside the IRES. Dashed boxes indicate unnecessary circularization elements.

FIG. 14 shows the migration mode of the purified products in the 2% E-Gel EX system for about 18 minutes. The lines indicate the product types of the corresponding bands, respectively. RNase R digests almost all the linear RNAs but not circular RNAs, allowing circular RNAs to be enriched.

FIG. 15 shows the effect of Mg²⁺ concentrations on circularization rate (% CircRNA in total-FA data) in IVT system; the dotted line indicates the 40% circularization rate.

FIG. 16 shows the migration mode of the purified products in the 2% E-Gel EX system for about 18 minutes. The lines indicate the product types of the corresponding bands, respectively. RNase R digestion allows circular RNAs to be enriched.

FIG. 17 shows the purity of the product with or without RNase R digestion detected by FA.

FIG. 18 shows the cell expression detection (FITC-GFP) of products prepared under different Mg²⁺ concentrations after treatment with RNase R.

FIG. 19 shows a schematic diagram of the circularization element with ribozyme P as an example according to some embodiments. The 5′ and 3′ recognizer sequences can simulate the formation of a P9.0 duplex mimic structure with at least two base pairs, including wobble base-pair G-U. Dashed boxes indicate nonessential circularization elements.

FIG. 20 shows the migration mode of the purified products in the 2% E-Gel EX system for about 18 minutes. The lines indicate the product types of the corresponding bands, respectively. RNase R digestion allows circular RNAs to be enriched.

FIG. 21 shows the purity of the product with or without RNase R digestion detected by FA.

FIG. 22 shows the cell expression detection (FITC-GFP) of products prepared under different Mg²⁺ concentrations after treatment with RNase R.

FIG. 23 shows a schematic diagram of the circularization element with ribozyme T as an example according to some embodiments, where the P9.2 is removed. Dashed boxes indicate nonessential circularization elements.

FIG. 24 shows the migration mode of the purified products in the 2% E-Gel EX system for about 18 minutes. The lines indicate the product types of the corresponding bands, respectively. RNase R digestion allows circular RNAs to be enriched.

FIG. 25 shows the purity of the product with or without RNase R digestion detected by FA.

FIG. 26 shows the cell expression detection (FITC-GFP) of products prepared under different Mg²⁺ concentrations before and after treatment with RNase R.

FIG. 27 shows a schematic diagram of the circularization elements with ribozyme T as an example according to some embodiments. The P1 duplex formed between the target site and IGS includes a C-A wobble base pair. Dashed boxes indicate nonessential circularization elements.

FIG. 28 shows the migration mode of the purified products in the 2% E-Gel EX system for about 18 minutes. The lines indicate the product types of the corresponding bands, respectively. RNase R digestion allows circular RNAs to be enriched.

FIG. 29 shows the purity of the product with or without RNase R digestion detected by FA.

FIG. 30 shows a schematic diagram of the circularization element with ribozyme T as an example according to some embodiments. The recognizers can also mediate circularization without P9.2 and homology arm elements.

FIG. 31 shows the migration mode of the purified products in the 2% E-Gel EX system for about 18 minutes. The lines indicate the product types of the corresponding bands, respectively. RNase R digestion allows circular RNAs to be enriched.

FIG. 32 shows a schematic diagram of the circularization element with ribozyme T as an example according to some embodiments. The 5′ and 3′ recognizer sequences can also mediate circularization without Spacers. Dashed boxes indicate nonessential circularization elements.

FIG. 33 shows the migration mode of the purified products in the 2% E-Gel EX system for about 18 minutes. The lines indicate the product types of the corresponding bands, respectively. RNase R digestion allows circular RNAs to be enriched. The product purity of FA analysis is presented in percentage form.

FIG. 34 shows the cell expression detection (FITC-GFP) of products prepared under different Mg²⁺ concentrations before and after treatment with RNase R.

FIG. 35 shows a schematic diagram of the circularization element with ribozyme P as an example according to some embodiments. The recognizers can also mediate circularization without homology arm elements and spacers. Dashed boxes indicate nonessential circularization elements.

FIG. 36 shows the migration mode of the purified products in the 2% E-Gel EX system for about 18 minutes. The lines indicate the product types of the corresponding bands, respectively. RNase R digestion allows circular RN to be enriched.

FIG. 37 shows a schematic diagram of the circularization element with ribozyme T as an example according to some embodiments. The recognizers can also mediate circularization without homology arm elements and linkers. Dashed boxes indicate nonessential circularization elements.

FIG. 38 shows the migration mode of the purified products in the 2% E-Gel EX system for about 18 minutes. The lines indicate the product types of the corresponding bands, respectively. RNase R digestion allows circular RNAs to be enriched. The product purity of FA analysis is presented in percentage form.

FIG. 39 shows the fluorescence image of GFP expression in Huh7 cells transfected with circularized samples (before and after RNase R treatment).

FIG. 40 shows a schematic diagram of the circularization element with ribozyme T as an example according to some embodiments. The recognizers cannot effectively mediate circularization when no paired structure is present. Dashed boxes indicate nonessential circularization elements.

FIG. 41 shows the migration mode of the purified products in the 2% E-Gel EX system for about 18 minutes. The lines indicate the product types of the corresponding bands, respectively.

FIG. 42 shows the fluorescence image of GFP expression in Huh7 cells transfected with circularized samples (before and after RNase R treatment).

FIG. 43 shows a schematic diagram of the circularization element with ribozyme T as an example according to some embodiments. The reintroduction of paired structures in R1 and R2 can restore circularization. Dashed boxes indicate nonessential circularization elements.

FIG. 44 shows the migration mode of the purified products in the 2% E-Gel EX system for about 18 minutes. The lines indicate the product types of the corresponding bands, respectively. RNase R digestion allows circular RNAs to be enriched. The product purity of FA analysis is presented in percentage form.

FIG. 45 shows the fluorescence image of GFP expression in Huh7 cells transfected with circularized samples (before and after RNase R treatment).

FIG. 46 shows a schematic diagram of the circularization element with ribozyme T as an example according to some embodiments. The 5′ and 3′ nucleotide sequences that constitute the native P9.0 duplex are swapped. Dashed boxes indicate nonessential circularization elements.

FIG. 47 shows the migration mode of the purified products in the 2% E-Gel EX system for about 18 minutes. The lines indicate the product types of the corresponding bands, respectively. RNase R digestion allows circular RNAs to be enriched. The product purity (CircRNA plus Nicked RNA, representing splicing efficiency) of FA analysis is presented in percentage form.

FIG. 48 shows the fluorescence image of GFP expression in Huh7 cells transfected with circularized samples (before and after RNase R treatment).

FIG. 49 shows a schematic diagram of the circularization element with ribozyme A as an example according to some embodiments. Dashed boxes indicate nonessential circularization elements.

FIG. 50 shows the migration mode of the purified products in the 2% E-Gel EX system for about 18 minutes. The lines indicate the product types of the corresponding bands, respectively. RNase R digestion allows circular RNAs to be enriched.

FIG. 51 shows the expression of firefly luciferase in A549 cells transfected with the circularized sample. Data are presented as relative luminescence units (RLU).

FIG. 52 shows a schematic diagram of the circularization element with ribozyme T as an example according to some embodiments. Dashed boxes indicate nonessential circularization elements.

FIG. 53 shows the migration mode of the purified products in the 2% E-Gel EX system for about 18 minutes. The lines indicate the product types of the corresponding bands, respectively. RNase R digestion allows circular RNAs to be enriched. The product purity (Circ plus Nicked, indicating splicing efficiency) of FA analysis is presented in percentage form.

FIG. 54 shows the protein expression detection (FITC-GFP) in Huh7 cells transfected with circularized samples (before and after RNase R treatment).

FIG. 55 shows a schematic diagram of the circularization element with ribozyme T as an example according to some embodiments. Arc with arrows indicates possible structural pairing between sequence elements. Dashed boxes indicate nonessential circularization elements.

FIG. 56 shows the migration mode of the purified products in the 2% E-Gel EX system for about 18 minutes. The lines indicate the product types of the corresponding bands, respectively. RNase R digestion allows circular RNAs to be enriched. The product purity (Circ plus Nicked, indicating splicing efficiency) of FA analysis is presented in percentage form.

FIG. 57 shows the protein expression detection (FITC-GFP) in Huh7 cells transfected with circularized samples (before and after RNase R treatment).

FIG. 58 shows a schematic diagram illustrating the two-step transesterification reaction of group I intron in self-splicing (adapted from Vicens Q. and Cech T. R., Trends Biochem Sci. 2006 January; 31 (1): 41-51.). (a) The 5′ splice site (marked by a conserved G·U pair) undergoes nucleophilic attack (arrow) by the 3′-OH group of a guanosine (or GMP or GTP) cofactor bound to the intron at the G-binding site. Lower- and uppercase characters stand for exon and intron sequences, respectively. (b) After the reaction, this guanosine is covalently linked to the 5′ end of the intron. (c) During a conformational change, the guanosine is displaced from the G-binding site by the 3′-terminal omega G (ΩG) that marks the 3′ splice site. The 3′-OH group of the terminal residue of the 5′exon attacks the 3′ splice site in a reaction that is chemically equivalent to the reverse of step 1. (d) The 5′ and 3′ exons are ligated and the intron is released. The group I intron is shown adopting its conserved secondary structure in black; the shaded box delimits its catalytic core.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless indicated otherwise, the scientific and technological terminologies used herein refer to meanings commonly understood by a person skilled in the art. Also, the terminologies and experimental procedures used herein relating to protein and nucleotide chemistry, molecular biology, cell and tissue cultivation, microbiology, immunology, all belong to terminologies and conventional methods generally used in the art. For example, the standard DNA recombination and molecular cloning technology used herein are well known to a person skilled in the art, and are described in details in the following references: Sambrook, J., Fritsch, Efland Maniatis, T., Molecular Cloning: A Laboratory Manual; Cold Spring Harbor Laboratory Press: Cold Spring Harbor, 1989. In the meantime, in order to better understand the present invention, definitions and explanations for the relevant terminologies are provided below.

Unless otherwise indicated, the term “at least” preceding a series of elements is to be understood to refer to every element in the series. Those skilled in the art will recognize or be able to ascertain using no more than routine experimentation, many equivalents to the specific embodiments of the present application. Such equivalents are intended to be encompassed by the present application.

As used herein, the expression “comprising”, “including”, “containing” or “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

It must be noted that as used herein and in the appended claims, the singular forms “a”, “an” and “the” include plural reference unless the context clearly dictates otherwise.

Unless otherwise stated, any numerical values, such as a concentration or a concentration range described herein, are to be understood as being modified in all instances by the term “about”. Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

The terms “polynucleotide”, “nucleic acid sequence”, “nucleotide sequence”, and “nucleic acid fragment” are used interchangeably to refer to a polymer of RNA or DNA that is single- or double-stranded, optionally containing synthetic, non-natural or modified nucleotides.

As used herein, a nucleotide in a nucleotide sequence is referred to by the single letter designation of its nucleobase as follows: “A (a)” for adenine or deoxyadenine (for RNA or DNA, respectively), “C (c)” for cytosine or deoxycytosine, “G (g)” for guanine or deoxyguanine, “U (u)” for uracil, “T (t)” for deoxythymine, “R” for purines (A or G), “Y” for pyrimidines (C or T), “I” for hypoxanthine, and “N” or “n” for any nucleotide. Although a nucleotide sequence may be represented as a DNA sequence (comprising T(s)), when referring to RNA, one skilled in the art can readily determine the corresponding RNA sequence (i.e., replacing T with U), and vice versa.

As used herein, “operably linked”, when referring to a first nucleotide sequence that is operably linked with a second nucleotide sequence, means a situation when the first nucleotide sequence is placed in a functional relationship with the second nucleotide sequence.

As used herein, the term “cis-splicing” refers to splicing from the same nucleic acid strand.

As used herein, the term “back-splicing site” or “backsplicing site” when used with reference to a circular RNA, refers to a dinucleotide served as the point of reconnection during the back-splicing process, resulting in the two ends of a linear nucleotide sequence joining to form the circular RNA.

As used herein, the term “splice site” refers to a dinucleotide between which a phosphodiester bond is cleaved during RNA circularization.

As used herein, the terms “native” and “naturally-occurring” mean the typical form of an organism, strain, gene or characteristic as it occurs in nature as distinguished from mutant or variant forms. For example, a naturally occurring group I intron or native nucleotide sequence of a group I intron may be present in and isolated from a natural source, and is not intentionally modified by human manipulation.

As used herein, the first nucleotide starting from the 5′ end of a nucleotide sequence is designated as the 5′ end nucleotide and is numbered as nucleotide 1 of the nucleotide sequence. Similarly, the last nucleotide starting from the 5′ end of a nucleotide sequence is designated as the 3′ end nucleotide of the nucleotide sequence.

As is understood by those skilled in the art, “upstream” is toward the 5′ direction of a nucleotide sequence and “downstream” is toward the 3′ direction of a nucleotide sequence.

Unless indicated otherwise, the expression “from 5′ end to 3′ end” means that the listed elements of a nucleotide sequence are present in a 5′ to 3′ direction and does not limit the length of the nucleotide sequence and elements therein. Thus, such an expression does not exclude any other elements located upstream, downstream and/or inbetween of the listed elements.

Unless indicated otherwise, a first nucleotide sequence (or a nucleotide) is “at the 5′ end” or “at the 3′ end” of a second nucleotide sequence refers to the terminal position of the first nucleotide sequence (or the nucleotide) within the second nucleotide sequence. While a first nucleotide sequence (or a nucleotide) is “in the 5′ region” or “in the 3′ region” of a second nucleotide sequence or a similar expression means the first nucleotide sequence (or the nucleotide) is located at a position adjacent to the 5′ or 3′ end nucleotide of the second nucleotide sequence but not necessarily at the 5′ or 3′ end of the second nucleotide sequence.

Secondary structures of RNAs can be predicted and/or determined from the nucleotide sequence by RNA structure prediction tools such as RNAfold (http://rna.tbi.univie.ac.at/cgi-bin/RNAWebSuite/RNAfold.cgi) or RNAstructure (https://rna.urmc.rochester.edu/RNAstructureWeb/index.html).

As used herein, the term “pair with” refers to two nucleic acid strands or two regions on the same nucleic acid strand form a duplex-containing structure through Watson-Crick base pairing and/or non-Watson-Crick base pairing. The expression “form”, “can form”, “may form” or the like is open-ended and inclusive, and do not exclude additional unrecited structures. For example, the expression “the 5′ and 3′ flanking sequences can pair with each to form a double-stranded region” means that a double-stranded region is formed through base pairing between at least a portion of the nucleotides in the 5′ and 3′ flanking sequences, but do not exclude any other structure may be formed by the 5′ flanking sequence and 3′ flanking sequence alone or in combination.

As used herein, the term “complementary” refers to Watson-Crick base pairing and/or non-Watson-Crick base pairing. As used herein, the term “reverse complementary” refers to base pairing is formed between a first nucleotide sequence in the 5′ to 3′ direction and a second nucleotide sequence in the 3′ to 5′ direction. Base pairings between two reverse complementary nucleotide sequences include Watson-Crick base pairing and/or non-Watson-Crick base pairing. Preferably, all base pairings between two reverse complementary nucleotide sequences are Watson-Crick base pairings. A “reverse complement” of a given nucleotide sequence can be obtained by reversing the order of all the nucleotides in the nucleotide sequence and then replacing all the nucleotides with their respective Watson-Crick complementary nucleotides.

The degree of complementarity between two nucleotide sequences can be indicated by the percentage of nucleotides in a first nucleotide sequence which can form hydrogen bonds (e.g., Watson-Crick base pairing) with a second nucleotide sequence (e.g., about 50%, about 60%, about 70%, about 80%, about 90%, and 100% complementary). Two nucleotide sequences are “reverse complementary” or “perfectly complementary” if all the contiguous nucleotides of a first nucleotide sequence form hydrogen bonds with the same number of contiguous nucleotides in a second nucleotide sequence.

As used herein, the term “at least partially (reverse) complementary” or “substantially complementary” means that at least about 50% (e.g., at least about 60%, at least about 65%, at least about 70%, at least about 75%, at least about 80%, at least about 85%, at least about 90%, at least about 95%, about 100%) nucleotides of a nucleotide sequence (e.g., a 5′ homology arm sequence) can form base pairs with another nucleotide sequence (e.g., a 3′ homology arm sequence). Two substantially complementary nucleotide sequences (for example, two homology arm sequences) may share a sufficient level of sequence identity to one another's reverse complement to allow hybridization occurs. Two nucleotide sequences (for example, two homology arm sequences) are “substantially complementary” or “at least partially complementary” if the two nucleotide sequences are at least 50% (e.g., at least 50%, at least 55%, at least 60%, at least 65%, at least 70%, at least 75%, at least 80%, at least 85%, at least 90%, at least 95%, at least 97%, at least 98%, at least 99%, or 100%) complementary over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleotide sequences hybridize under at least moderate, or, in some embodiments high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook, J., Molecular Cloning: A Laboratory Manual, Cold Spring Harbor Laboratory Press; 4th edition (Jun. 15, 2012). High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (optionally in combination with EDTA). Additional details and an explanation of stringency of hybridization reactions are provided in, e.g., Sambrook, supra; and Ausubel et al., eds., Short Protocols in Molecular Biology, 5th ed., John Wiley & Sons, Inc., Hoboken, N.J. (2002).

As used herein, two “homology arm sequences” or “homology arms” complement, or are complementary, to one another when the two regions share a sufficient level of sequence identity to one another's reverse complement to allow hybridization occurs. As used herein, a “homology arm sequence” is any contiguous sequence that can form base pairs with preferably at least about 50% (e.g., at least about 60%, at least about 65%, at least about 70%, at least about 75%, at least about 80%, at least about 85%, at least about 90%, at least about 95%, about 100%) of another sequence (another homology arm sequence) in the RNA construct.

As used herein, a “spacer” refers to a nucleotide sequence separating two other elements (segments) along a polynucleotide sequence. A spacer may be of any length. For example, A spacer may be of 1-100 nucleotides, preferably 2-50 nucleotides in length. A spacer may comprise a defined or random nucleotide sequence.

As used herein, the term “Watson-Crick base pairing” refers to a hydrogen-bond pairing occurs between adenine and thymine (A-T) (DNA) or uracil (A-U) (RNA), or guanine and cytosine (G-C).

As used herein, the term “wobble base pairing” refers to a type of non-Watson-Crick base pairing. Wobble base pairing may be formed between hypoxanthine and uracil (I-U, I for inosine), guanine and uracil (G-U), adenine and cytosine (A-C), hypoxanthine and adenine (I-A), or hypoxanthine and cytosine (I-C), but not limited to.

As used herein, the term “base pair” or “base pairing” refers to two nitrogenous bases that are connected by hydrogen bonds. A base pair can be a Watson-Crick base pair or a non-Watson-Crick base pair. Examples of non-Watson-Crick base pairs may include but not limited to wobble base pairs and Hoogsteen base pairs. Among the most frequent of wobble base pairs are G-T (U) base pairing and A-C base pairing. Other non-Watson-Crick base pairs include but are not limited to C-U, A-G (or I) and A-A.

As used herein, the term “stem-loop”, also known as a “hairpin”, refers to a secondary structure that can occur in single-stranded nucleic acids. The stem-loop may occur when two regions of the same strand pair with each other to form a double-stranded region that ends in an unpaired loop.

As used herein, the terms “duplex”, “double-stranded region” and “helix” are used interchangeably to refer to a double-stranded structure comprising at least one base pair. A duplex may comprise a Watson-Crick base pair, a non-Watson-Crick base pair or a combination thereof.

As used herein, the term “duplex mimic” refers to a double-stranded structure that functionally mimics the native duplex structure of a group I intron ribozyme. A duplex mimic may comprise at least one base pair. A duplex mimic may comprise a Watson-Crick base pair, a non-Watson-Crick base pair or a combination thereof. The sequences forming a duplex mimic preferably are but not limited to the corresponding native ribozyme sequences and can be truncated or designed as other alternative sequences.

As used herein, the term “free energy” refers to the energy released by folding an unfolded nucleic acid molecule (e.g., RNA or DNA, etc.), or, conversely, the amount of energy that must be added in order to unfold a folded nucleic acid molecule (e.g., RNA or DNA, etc.). The “minimum free energy (MFE)” of a nucleic acid molecule (e.g., DNA, RNA, etc.) describes the lowest value of free energy observed for the nucleic acid molecule when assessed for various secondary structures thereof. The more negative free energy a structure has, the more likely is its formation.

As used herein, the term “melting temperature (Tm)” refers to the temperature at which about 50% of double-stranded nucleic acid structures (e.g., DNA/DNA, DNA/RNA, or RNA/RNA duplexes) denature and dissociate to single-stranded structures. The melting temperature of a particular nucleic acid molecule can be determined using thermodynamic analyses and algorithms described herein and known in the art (see, e.g., Kibbe W. A., Nucleic Acids Res., 35 (Web Server issue): W43-W46 (2007). doi: 10.1093/nar/gkm234; and Dumousseau et al., BMC Bioinformatics, 13:101 (2012). doi.org/10.1186/1471-2105-13-101).

When referring to a nucleotide sequence or protein sequence, the term “identity” is used to denote similarity between two sequences. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection. Another algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

As used herein, a “recombinant” nucleic acid (e.g., a recombinant group I intron) refers to a non-naturally occurring nucleic acid resulted from artificial modifications, such as mutagenesis that is distinguishable from naturally occurring nucleic acids found in natural sources.

As used herein, definitions of IGS and the paired regions, for example, P1, P2, P4-P6, P3-P9, P9.0, P9a, P9b, P9.1, P9.1a, P9.2 and P10 duplexes and P1 extension, of a group I intron are known in the art and can be determined, for example, with reference to the following documents: Burke J. M. et al., Structural conventions for group I introns. Nucleic Acids Res. 1987 September; 15 (18): 7217-21; Stahley M. R. and Strobel S. A., RNA splicing: group I intron crystal structures reveal the basis of splice site selection and metal ion catalysis, Current Opinion in Structural Biology, 2006 16 (3): 319-326; and Woodson S. A. Structure and assembly of group I introns. Curr Opin Struct Biol. 2005 15 (3): 324-30. Representative sequences and secondary structures of group I introns are available, for example, on website https://crw2-comparative-rna-web.org/group-i-introns/.

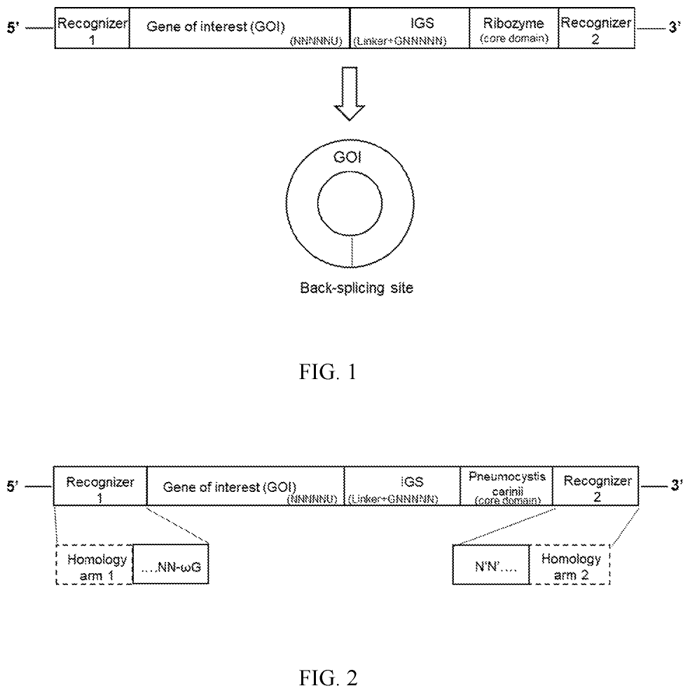

The essential sequence elements of the novel cis-splicing mediated RNA circularization system based on group I introns are shown in FIG. 1.

The nucleotide sequence of interest comprises a target site (e.g., ‘NNNNNU’) that can pair with the interanl guide sequence (IGS) (e.g., ‘GNNNNN’) to determine the 5′ splice site. The 5′ recognizer sequence (R1) comprises a first pairing sequence and a 3′ end nucleotide ‘N’ (also referred to as “ωN”). In some particular embodiments, the 3′ end nucleotide ‘N’ is guanine (also referred to as “ωG”). The 3′ recognizer sequence (R2) comprises a second pairing sequence that can pair with the first pairing sequence to form a duplex which helps to determine the 3′ splice site downstream the ωN. In some particular embodiments, the ribozyme core is capable of catalyzing the formation of a circular RNA comprising the nucleotide sequence of interest by joining the nucleotide immediately downstream the ωN (i.e., the nucleotide at the ωN+1 position in the RNA construct) and the 3′ end nucleotide of the target site (e.g., the 3′ end ‘U’ if the target site is ‘NNNNNU’).

R1 may further comprise a 5′ flanking sequence. R2 may further comprise a 3′ flanking sequence. The 5′ and 3′ flanking sequences may pair with each other to form a double-stranded region which promotes the 5′ and 3′ ends of the RNA construct to be close and thereby helping to determine the duplex required for the 3′ splice site.

In one aspect, the disclosure provides an RNA construct (Construct 1) comprising, from 5′ end to 3′ end:
a first recognizer sequence (R1) comprising a first pairing sequence;
a nucleotide sequence of interest (GOI) comprising a target site at its 3′ end;
a ribozyme core sequence operably linked to an internal guide sequence (IGS), wherein the ribozyme core sequence encodes a ribozyme core having the catalytic activity of a group I intron ribozyme; and
a second recognizer sequence (R2) comprising a second pairing sequence substantially complementary to the first pairing sequence;
wherein
the 5′ end nucleotide of the IGS and the 3′ end nucleotide of the target site form a non-Watson-Crick base pair to define a 5′ splice site;
R1 and R2 are positioned at opposite ends of the RNA construct, such that hybridization of the first and second pairing sequences results in formation of a duplex-containing structure to define a 3′ splice site;
the GOI is positioned 5′ to the ribozyme core sequence and IGS; and
the RNA construct is capable of generating a circular RNA comprising the nucleotide sequence of interest through the catalytic activity of the ribozyme core.

In some embodiments, the RNA construct comprises, from 5′ end to 3′ end,
R1 comprising a first pairing sequence and a 3′ end nucleotide ‘N’ (ωN);
GOI comprising a target site at its 3′ end;
IGS;
Ribozyme core sequence; and
R2 comprising a second pairing sequence;
wherein
ωN is any naturally occurring or modified nucleotide;
the 5′ end nucleotide of the IGS and the 3′ end nucleotide of the target site form a non-Watson-Crick base pair to define the 5′ splice site; and
the first and second pairing sequences are substantially complementary to form a duplex-containing structure upstream of the ωN to define the 3′ splice site.

In some embodiments, ωN is guanine (ωG).

Group I introns may be categorized into 14 subgroups including IA1-3, IB1-4, IC1-3, ID and IE1-3. The self-splicing group I intron useful in the present disclosure may be obtained or derived from any organism, such as, for example, fungi, bacteria, bacteriophages, and eukaryotic viruses. Examples of group I introns useful in the present disclosure include, but are not limited to, group I introns derived from the following organisms: Enterobacteria phage T4, Bacteriophage Twort, Bacteriophage SPO1, Bacteriophage S3b, Bacillus anthracis, Clostridium botulinum, Tetrahymena thermophile (e.g., Ttch.L1925), Didymium iridis (e.g., Dir.S956-2), Diderma niveum, Dunaliella parva, Pneumocystis carinii, Physarum polycephalum (e.g., Ppo.L1925), Anabaena sp. PCC7120, Scytonema hofmanni, Agrobacterium tumefaciens, Synechocystis sp. PCC 6803, Synechococcus elongatus PCC 6301, Neurospora crassa, Candida albicans, Scytalidium cerradiumydiaces, Scytalidium dimidiatum, Pediadiaces Chlamydomonas nivalis, Chlorella vulgaris, Amoebidium parasiticum, Pediastrum biradiatum, Emericella nidulans, Saccharomyces cerevisiae, Schizosaccharomyces pombe, Azoarcus sp. BH72, Neochloris aquatica, and Symkania negevensis. See e.g., Vicens Q. et al., Toward predicting self-splicing and protein-facilitated splicing of group I introns. RNA. 2008 October; 14 (10): 2013-29; Tanner M. and Cech T., Activity and thermostability of the small self-splicing group I intron in the pre-tRNA (11e) of the purple bacterium Azoarcus. RNA. 1996 January; 2 (1): 74-83; Vicens Q. and Cech T. R., Atomic level architecture of group I introns revealed. Trends Biochem Sci. 2006 January; 31 (1): 41-51.; Hedberg A. and Johansen S. D., Nuclear group I introns in self-splicing and beyond. Mob DNA. 2013 Jun. 5; 4 (1): 17. A group I intron can be a naturally occurring or a recombinant group I intron. A recombinant group I intron can be obtained, for example, by deleting, inserting and/or substituting one or more nucleotides of a naturally occurring group I intron, as long as the self-splicing activity is retained.

The ribozyme core has the catalytic activity of a group I intron ribozyme means that the ribozyme core is able to catalyze self-splicing of the RNA construct with the help of IGS/target site determining the 5′ splice site, and the 5′ recognizer sequence (R1)/the 3′ recognizer sequence (R2) and ωN (e.g., ωG in some embodiments) determining the 3′ splice site like a group I intron ribozyme. In some embodiments, the resulting circular RNA is formed by connecting the 3′ end nucleotide of the target site and the nucleotide immediately downstream of the ωN (e.g., ωG in some embodiments).

In some embodiments, the ribozyme core sequence comprises a nucleotide sequence encoding the scaffold domain and catalytic domain of a group I intron. The scaffold domain may comprise P4-P6 (P4, P5 and P6) and the catalytic domain may comprise P3-P8 (P3, P7 and P8). In some embodiments, the ribozyme core sequence comprises or consists of the sequence from the IGS end (e.g., starting from a nucleotide downstream (e.g., immediately downstream) of the 3′ end nucleotide of the IGS) to the sequence before the P9.0 duplex (i.e., before the 5′ half of P9.0 duplex) of a group I intron.

In some embodiments, the ribozyme core sequence does not comprise the sequence for the P9.0 duplex of a group I intron. In some embodiments, the ribozyme core sequence does not comprise the sequence from the 5′ half of P9.0 duplex to the 3′ end nucleotide of a group I intron. In some embodiments, the ribozyme core sequence comprises the complete sequence between P1-P9.0 duplexes of a group I intron, excluding the sequences for the P1 and P9.0 duplexes. For example, in embodiments wherein the ribozyme core sequence is derived from a Pneumocystis carinii or Tetrahymena sp. group I intron, the ribozyme core sequence may comprise or consist of the sequence from the IGS end to the sequence before the P9.0 duplex (i.e., before the 5′ half of P9.0 duplex) of the group I intron.

The ribozyme core sequence may be derived from any group I intron, including but not limited to the group I introns as described above. In some embodiments, the ribozyme core sequence is derived from a group IC1 (e.g., from Tetrahymena sp. (e.g., T. thermophile, T. cosmopolitanis (e.g., GenBank accession number: X03107), T. hyperangularis (e.g., GenBank accession number: X03106), T. malaccensis (e.g., GenBank accession number: X03105) or T. pigmentosa (e.g., GenBank accession number: J01210)) or Pneumocystis sp. (e.g., Pneumocystis carinii), IC2, IC3 (e.g., from Anabaena sp. PCC7120 (e.g., GenBank accession number: M38692) or Azoarcus sp. BH72 (e.g., GenBank accession number: X66221)) or IA2 (e.g., from Bacteriophage Twort) intron. The group I intron from which the ribozyme core sequence is derived can be a naturally occurring group I intron or a recombinant group I intron. In some embodiments, the ribozyme core sequence comprises a nucleotide sequence having at least 80%, 85%, 90%, 91%, 92%, 93%, 94%, 95%, 96%, 97%, 98%, 99% or 100% sequence identity to the nucleotide sequence of a naturally occurring group I intron.

In some embodiments, the ribozyme core sequence is derived from a Pneumocystis sp. group I intron, e.g., a Pneumocystis sp. group I intron comprising a nucleotide sequence selected from SEQ ID NOs: 32-36. In a particular embodiment, the ribozyme core sequence is derived from a Pneumocystis carinii group I intron comprising the nucleotide sequence of SEQ ID NO: 32. In a particular embodiment, the ribozyme core sequence comprises or consists of the nucleotide sequence of SEQ ID NO: 19 or a nucleotide sequence having at least 95% sequence identity thereto.

In some embodiments, the ribozyme core sequence is derived from a Tetrahymena sp. group I intron, e.g., a Tetrahymena thermophile group I intron comprising the sequence of SEQ ID NO: 12. In a particular embodiment, the ribozyme core sequence comprises or consists of the nucleotide sequence of SEQ ID NO: 17 or a nucleotide sequence having at least 95% sequence identity thereto.

In some embodiments, the ribozyme core sequence is derived from an Anabaena sp. group I intron; for example, the ribozyme core sequence comprises or consists of the nucleotide sequence of SEQ ID NO: 48 or a nucleotide sequence having at least 95% sequence identity thereto.

The first and second pairing sequences can pair with each other to form a duplex-containing structure upstream of the ωN to define a 3′ splice site downstream the ωN. The duplex-containing structure may comprise at least one base pair and have a minimum free energy (MFE) of less than −18.9 KJ/mol and a melting temperature of at least 35.0° C. The free energy parameters can be determined using any method known in the art, for example, an RNA secondary structure predicting tool such as RNAfold and RNAstructure, or by experimental methods such as optical melting experiments, in conjunction with NMR or crystallography. Algorithms for determining MFE are further described in, e.g., Hajiaghayi et al., BMC Bioinformatics, 13:22 (2012); Mathews, D. H., Bioinformatics, Volume 21, Issue 10:2246-2253 (2005); and Doshi et al., BMC Bioinformatics, 5:105 (2004) doi 10.1186/1471-2105-5-105). Alternatively, the formation of a duplex-containing structure between the first and second pairing sequences can be predicted by determining the optimal secondary structure of the RNA construct of the present disclosure.

The most commonly used software programs, employed to predict the secondary RNA or DNA structures by MFE algorithms, make use of the so-called nearest-neighbor energy model. This model uses free energy rules based on empirical thermodynamic parameters (Mathews et al., J Mol Biol, 288:911-940 (1999); and Mathews et al., Proc Natl Acad Sci USA, 101:7287-7292 (2004)) and computes the overall stability of an RNA or DNA structure by adding independent contributions of local free energy interactions due to adjacent base pairs and loop regions.

In some embodiments, the duplex-containing structure may have a minimum free energy (MFE) of less than about −18.9 KJ/mol (e.g., less than about −17 KJ/mol, less than about −18 KJ/mol, less than about −18.9 KJ/mol, less than about −19 KJ/mol, less than about −20 KJ/mol, less than about −30 KJ/mol, less than about −40 KJ/mol). In some embodiments, the MFE is greater than about −90 KJ/mol (e.g., greater than about −85 KJ/mol, greater than about −80 KJ/mol, greater than about −70 KJ/mol, greater than about −60 KJ/mol, greater than about −50 KJ/mol, greater than about −40 KJ/mol). In some embodiments, the duplex-containing structure has a minimum free energy (MFE) of about −18.9 KJ/mol or less. In some embodiments, the duplex-containing structure has an MFE in the range of about −18.9 kJ/mol to about −90 KJ/mol.

In some embodiments, the duplex-containing structure has a melting temperature of at least 35.0° C. In some embodiments, the duplex-containing structure has a melting temperature of at least 35.0° C., but not more than about 85° C. In some embodiments, the RNA secondary structure has a melting temperature of at least 35° C., 36° C., 37° C., 38° C., 39° C., 40° C., 41° C., 42° C., 43° C., 44° C., 45° C., 46° C., 47° C., 48° C., 49° C. or greater. In some embodiments, the melting temperature is no more than about 85° C., no more than about 75° C., no more than about 70° C., no more than about 65° C., no more than about 60° C., no more than about 55° C., no more than about 50° C. or less.

The duplex-containing structure may comprise one or more base pairs, e.g., 1-200, 1-50, 5-45, 10-40, 15-35, 15-20, 3, 4, 5, 6, 7, 8, 9, 10, 11, 12, 13, 14, 15, 16, 17, 18, 19 or 20 base pairs, consecutive or interrupted by one or more mismatches. In some embodiments, the duplex-containing structure comprises at least two base pairs. In some preferable embodiments, the duplex-containing structure comprises at least two consecutive base pairs. For example, the duplex-containing structure may comprise 2-100, 3-80, 5-60, 5, 10, 11, 12, 13, 14, 15, 16, 17, 18, 19, 20, 21, 22, 23, 24, 25, 26, 27, 28, 29, 30, 35, 40, 45 or 50 consecutive base pairs. In some embodiments, at least one base pair is located immediately upstream of the ωN. In some preferable embodiments, 2-6 consecutive base pairs of the duplex-containing structure are located immediately upstream of the ωN. Examples of duplex-containing structures may include but are not limited to stem structures, stem-loop structures and stem-loop alternating structures.

The duplex-containing structure may comprise a Watson-Crick base pair, a non-Watson-Crick base pair or a combination thereof. The duplex-containing structure may comprise a base pair selected from A-U, G-C, G-A, A-A, U-U, A-C, G-U and a combination thereof. The duplex-containing structure may optionally comprise one or more structures selected from a bulge loop, an interior loop and a hairpin loop.

The first and second pairing sequences may independently comprise 1-100 nucleotides, for example, 2-90, 5-90, 10-80, 20-60, 30-50, 40-45, 2, 4, 5, 6, 7, 8, 9, 10, 11, 12, 15, 18, 20, or 25 nucleotides. In some embodiments, the first pairing sequence comprises 2-20, 2-12, 4-10, 6, 7 or 8 nucleotides. In some embodiments, the second pairing sequence comprises 2-100, 5-80, 8-60, 10-50, 15, 20, 30, 40, 50, 60, 70, 80, 90 or 100, preferably 5-80 or 8-60 nucleotides. The first and second pairing sequences may share a sufficient level of sequence identity to one another's reverse complement to allow the 5′ and 3′ ends of the RNA construct to form the duplex-containing structure. The percent sequence identity can be any percent of sequence identity that allows for hybridization to occur. In some embodiments, at least 50%, 60%, 65%, 70%, 75%, 80%, 85%, 90%, 95%, 96%, 97%, 98%, 99% or 100% nucleotides of the first pairing sequence form base pairs with the second pairing sequence. In some embodiments, the first pairing sequence is at least 50%, at least 55%, at least 60%, at least 65%, at least 70%, at least 75%, at least 80%, at least 85%, at least 90%, at least 95%, at least 97%, at least 98%, at least 99%, or 100% complementary to the second pairing sequence.

In some embodiments, the first pairing sequence comprises a sequence of at least 2 contiguous nucleotides, for example, a sequence of 2-100 contiguous nucleotides which is reverse complementary to a sequence of the same number of contiguous nucleotides in the second pairing sequence. In some preferable embodiments, the first pairing sequence comprises a sequence of 2-6 contiguous nucleotides, which is reverse complementary to a sequence of the same number of contiguous nucleotides in the second pairing sequence.

The base pairs formed between the first and second pairing sequences may be located anywhere upstream of the ωN, preferably upstream and adjacent to the ωN, for example, immediately upstream of the ωN (for example, at least one base pair is located at the ωN−1 position in the RNA construct), or located a few (e.g., 1-50, 10-40, 20-30, 1, 2, 3, 4, 5, 6, 7, 8, 9, 10, 11, 12, 13, 14, 15, 16, 17, 18, 19 or 20) nucleotides upstream of the ωN (for example, at least one base pair is located at the ωN−2, ωN−3, ωN−4, ωN−5, ωN−6, ωN−7, ωN−8, ωN−9 or ωN−10 position in the RNA construct). In some embodiments, ωN is guanine (ωG). As demonstrated in some embodiments of the present application, one or more base pairs formed in close proximity of the ωN, mimicking the P9.0 duplex in a native group I intron, are essential for higher circularization efficiency and more accurate splicing. Accordingly, in some preferable embodiments, the relative location of the duplex formed to the ωN in the RNA construct is substantially identical to that of the P9.0 duplex to the ωG in the group I intron from which the ribozyme core sequence is derived.

In some preferable embodiments, the first and second pairing sequences form at least one base pair upstream and adjacent to the ωN, such that base pairing between the first and second pairing sequences simulate the formation of a P9.0 duplex upstream the ωG in the native group I intron during the circularization reaction. The duplex formed adjacent to the ωN may be also referred to as a “P9.0 duplex mimic”.

The P9.0 duplex mimic may comprise at least one base pair. For example, the P9.0 duplex mimic may comprise 1, 2, 3, 4, 5, 6, 7, 8, 9 or 10 base pairs. Preferably, the P9.0 duplex mimic may comprise 2-6 consecutive base pairs. Preferably, the P9.0 duplex mimic comprises a substantially identical number of base pairs to that of the P9.0 duplex of the group I intron from which the ribozyme core sequence is derived. Those skilled in the art would be able to determine essential features for a P9.0 duplex mimic in view of the present disclosure and the prior art.

The P9.0 duplex mimic may comprise a Watson-Crick base pair, a non-Watson-Crick base pair or a combination thereof. In some embodiments, the non-Watson-Crick base pair is a wobble base pair. A preferable example of a non-Watson-Crick base pair may be a G-U wobble base pair. In a particular embodiment, the ribozyme core sequence is derived from a Pneumocystis carinii group I intron, the P9.0 duplex mimic may comprise a G-U wobble base pair.

In some embodiments, the first pairing sequence comprises a nucleotide ‘N₁’ that is able to form a base pair with a nucleotide ‘n₁’ of the second pairing sequence, wherein ‘N₁’ is located at an ωN-i position in the RNA construct, i is an integer of 1-21. In some particular embodiments, i is an integer of 1-11. In some preferable embodiments, i is 1 or 2.

In some embodiments, ‘N₁’ is the 3′ end nucleotide of a first contiguous sequence of 2-6 nucleotides in the first pairing sequence, ‘n₁’ is the 5′ end nucleotide of a second contiguous sequence in the second pairing sequence, wherein the first contiguous sequence is reverse complementary to the second contiguous sequence.

In some embodiments, ‘N₁’ is the 3′ end nucleotide of a first contiguous sequence of 2-6 nucleotides in the first pairing sequence, ‘n₁’ is the 5′ end nucleotide of a second contiguous sequence in the second pairing sequence, wherein the first contiguous sequence is reverse complementary to the second contiguous sequence, and i is an integer of 1-21. In some particular embodiments, i is an integer of 1-11. In some preferable embodiments, i is 1 or 2.

In some embodiments, R1 comprises a nucleotide sequence ‘(Nₓ)ₛ(N_{y})ₜ(ωN)’ at its 3′ end, and R2 comprises a nucleotide sequence ‘(nₓ)_{w}’; wherein, ωN, ‘Nₓ’, ‘nₓ’ and ‘N_{y}’ are each independently any naturally occurring or modified nucleotide, t is an integer of 0-20, s and w are each independently an integer of 1-200, and ‘(Nₓ)ₛ’ and ‘(nₓ)_{w}’ are substantially complementary to form a duplex-containing structure upstream of the ωN to define the 3′ splice site.

In some embodiments, ωN is guanine (ωG).

In some embodiments, t is an integer of 0-10. In some preferable embodiments, t is 0. In some other embodiments, t is 1.

In some embodiments, each of s and w is an integer of h which is selected from 2-6, ‘(Nₓ)ₕ’ and ‘(nₓ)ₕ’ are reverse complementary, and t is 0-20. In some particular embodiments, the ribozyme core sequence is derived from a group IC1 intron, for example, an Tetrahymena sp. (e.g., T. thermophile) or Pneumocystis sp. group I intron, and t is an integer of 0-20. In some particular embodiments, the ribozyme core sequence comprises or consists of the nucleotide sequence of SEQ ID NO: 17 or SEQ ID NO: 19 or a nucleotide sequence having at least 95% sequence identity thereto, and t is 0.

In some embodiments, R1 comprises a nucleotide sequence ‘N₁(N_{y})ₜG’ at its 3′ end, and R2 comprises a nucleotide ‘n₁’, wherein ‘G’ is the ωG, ‘N₁’, ‘n₁’ and ‘N_{y}’ are each independently any naturally occurring or modified nucleotide, t is an integer of 0-20, and ‘N₁’ and ‘n₁’ form a base pair. In some embodiments, t is 0. In some embodiments, the ribozyme core sequence is derived from a group IC1 intron, for example, an Tetrahymena sp. (e.g., T. thermophile) or Pneumocystis sp. group I intron, and t is 0.

In some other embodiments, t is an integer of 1-20, for example, 1, 2, 3, 4, 5, 6, 7, 8, 9, 10, 11, 12, 13, 14, 15, 16, 17, 18, 19 or 20. In some particular embodiments, for example, wherein the ribozyme core sequence is derived from a group IC3 intron, for example, an Azoarcus sp. group I intron (e.g., derived from Azoarcus sp. strain BH72), t is 1. In some particular embodiments, the ribozyme core sequence is derived from a group IC1 intron, for example, an Tetrahymena sp. (e.g., T. thermophile) group I intron, t is an integer of 1-10, preferably t is 1.

In some embodiments, R1 comprises a nucleotide sequence ‘N₂N₁ (N_{y})ₜ(G’ at its 3′ end, and R2 comprises a nucleotide sequence ‘n₁n₂’, wherein ‘G’ is the ωG, ‘N₁’, ‘n₁’, ‘N₂’, ‘n₂’ and ‘N_{y}’ are each independently any naturally occurring or modified nucleotide, t is an integer of 0-20, ‘N₁’ and ‘n₁’ form a first base pair, and ‘N₂’ and ‘n₂’ form a second base pair. In some embodiments, t is 0, R1 comprises a nucleotide sequence ‘N₂N₁G’ at its 3′ end. In some other embodiments, for example, wherein the ribozyme core sequence is derived from a group IC3 intron (e.g., an Azoarcus sp. or Annona cherimola group I intron), t is 1, R1 comprises a nucleotide sequence ‘N₂N₁N_{y}G’, wherein ‘N_{y}’ is any naturally occurring or modified nucleotide; for example, ‘N_{y}’ is ‘G’, ‘U’ or ‘A’. In some embodiments, the first and second base pairs are each selected from A-U, G-C, G-A, A-A, U-U, A-C, G-U and a combination thereof.

In some embodiments, the 5′ recognizer sequence (R1) may further comprise a 5′ flanking sequence located upstream of the first pairing sequence. In some embodiments, the 3′ recognizer sequence (R2) may further comprise a 3′ flanking sequence located downstream of the second pairing sequence. The 5′ flanking sequence and 3′ flanking sequence may pair with each other to form at least one RNA secondary structure that promotes the 5′ and 3′ ends of the RNA construct to be close. The at least one RNA secondary structure may comprise a double-stranded region formed by base pairing between the 5′ and 3′ flanking sequences, and optionally one or more structures selected from a bulge loop, an inteior loop and a hairpin loop. Examples of such RNA secondary structures include but are not limited to stem structures, stem-loop structures and stem-loop alternating structures. The 5′ and 3′ flanking sequences each may independently comprise 1-500 nucleotides, for example, 10-500, 20-400, 30-300, 40-200, 50-100, 60-90 or 70-80 nucleotides. In some embodiment, the 5′ and 3′ flanking sequences each independently comprises 3-400, 4-200, 5-150, 10-100 or 20-50 nucleotides.

The double-stranded region may comprise one or more base pairs, e.g., about 2-500, about 5-100, about 2-50, about 10-50 or about 20-30 base pairs, consecutive or interrupted by one or more mismatches. Preferably, the double-stranded region comprises 2-50 base pairs, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 base pairs. Preferable examples of the 5′ and 3′ flanking sequences may be homology arm sequences. For example, a double-stranded region can be formed by two homology arm sequences that are substantially reverse complementary.

In some embodiments, the 5′ flanking sequence comprises a 5′ homology arm sequence, and the 3′ flanking sequence comprises a 3′ homology arm sequence, and the 5′ and 3′ homology arm sequences are substantially complementary. In some embodiments, R1 further comprises a 5′ homology arm sequence located upstream of the first pairing sequence and R2 further comprises a 3′ homology arm sequence located downstream of the second pairing sequence, wherein the 5′ and 3′ homology arm sequences are substantially complementary. The 5′ and 3′ homology arm sequences each may independently comprise 5-50 nucleotides, for example, 5, 6, 7, 8, 9, 10, 15, 20, 25, 30, 35, 40, 45 or 50 nucleotides. In an embodiment, the 5′ and 3′ homology arm sequences are reverse complementary. In another embodiment, the 5′ and 3′ homology arm sequences are partially reverse complementary, for example, at least 50%, 60%, 65%, 70%, 75%, 80%, 85%, 90% or 95% or 99% nucleotides of the 5′ and 3′ homology arm sequences form base pairs. Preferably, the 5′ and 3′ homology arm sequences share a higher percent of identity to one another's reverse complement than they to a sequence located within the GOI and/or the ribozyme core sequence, such that formation of a double-stranded region between the 5′ and 3′ homology arm sequences is prioritized.

In some embodiments, the 5′ and 3′ flanking sequences, alone or in combination, may form one or more structures mimicking the native structures of the group I intron ribozyme. For example, in embodiments wherein the ribozyme core sequence is derived from a Tetrahymena sp. group I intron, the 5′ and 3′ flanking sequences, alone or in combination, may form one or more structures mimicking the native P9 (P9a/9b), P9.1, P9.1a or P9.2 duplex of the group I intron or a combination thereof. Preferably, the 5′ and 3′ flanking sequences in combination form a structure mimicking the P9.2 duplex of the group I intron.

The RNA construct according to the present disclosure can be derived from a group I intron by inserting a nucleotide sequence of interest between a 3′ fragment (corresponding to R1) and a 5′ fragment (corresponding to Ribozyme core-R2) of a group I intron, wherein the 3′ fragment and 5′ fragment in combination retain the self-splicing ability of the group I intron. Upon further investigation, the present inventor unexpectedly discovered that a 3′ end portion (e.g., a sequence from the 5′ half of P9.0 duplex to the 3′ end nucleotide) of a group I intron could be deleted and modified without disrupting the catalytic activity of the group I intron and the formation of a duplex-containing structure comprising any sequence between the 5′ and 3′ ends of the RNA construct is only required to facilitate circularization through the self-splicing activity of the ribozyme core.

Accordingly, the present disclosure provides, in another aspect, an RNA construct (Construct 2) comprising, from 5′ end to 3′ end,
a first nucleotide sequence comprising a sequence from a nucleotide ‘N_{q}’ to the 3′ end of a group I intron,
a nucleotide sequence of interest (GOI) comprising a target site at its 3′ end,
an internal guide sequence (IGS), and
a second nucleotide sequence comprising a sequence from the IGS end to a nucleotide ‘Nₚ’ of a group I intron;
wherein
the 5′ end nucleotide of the IGS and the 3′ end nucleotide of the target site form a non-Watson-Crick base pair to define a 5′ splice site;
‘Nₚ’ and ‘N_{q}’ are independently selected from any nucleotide from the 3′ end nucleotide of the 5′ half to the 5′ end nucleotide of the 3′ half of P9.0 duplex of the group I intron, and
‘Nₚ’ is located upstream of ‘N_{q}’ in the group I intron.

The group I intron can be a group I intron as described above. Preferably, the group I intron is a group IC1 (e.g., from Tetrahymena sp. (e.g., T. thermophile, T. cosmopolitanis, T. hyperangularis, T. malaccensis or T. pigmentosa) or Pneumocystis sp. (e.g., Pneumocystis carinii), IC2, IC3 (e.g., from Anabaena sp. PCC7120 or Azoarcus sp. BH72) or IA2 (e.g., from Bacteriophage Twort) intron. In an embodiment, the group I intron is a Pneumocystis sp. group I intron comprising a nucleotide sequence selected from SEQ ID NOs: 32-36. In another embodiment, the group I intron is a Tetrahymena thermophila group I intron comprising the nucleotide sequence of SEQ ID NO: 12. In another embodiment, the group I intron is an Anabaena sp. group I intron comprising the nucleotide sequence of SEQ ID NO: 49.

‘Nₚ’ and ‘N_{q}’ are selected such that a P9.0 duplex mimic can be formed between R1 and R2. The first and second nucleotide sequences in combination retain the self-splicing ability of the group I intron, but not necessarily constitute the full-length of the group I intron. For example, the first and second nucleotide sequences in combination may lack one or more duplexes that is not a P9.0 duplex in the P9 domain of the group I intron. For example, the first and second nucleotide sequences in combination may lack one or more duplexes selected from a P9a duplex, a P9b duplex, a P9.1 duplex, a P9.1a duplex and a P9.2 duplex, when applicable. Preferably, the first and second nucleotide sequences in combination comprise at least one duplex selected from a P9a duplex, a P9b duplex, a P9.1 duplex, a P9.1a duplex and a P9.2 duplex, when applicable.

In some embodiments, the group I intron is a Pneumocystis carinii group I intron comprising the nucleotide sequence of SEQ ID NO: 32, ‘Nₚ’ and ‘N_{q}’ are independently selected from any nucleotide from nucleotide 316 (U316) to nucleotide 342 (G342) of SEQ ID NO: 32. In some embodiments, the group I intron is a Tetrahymena thermophila group I intron comprising the nucleotide sequence of SEQ ID NO: 12, ‘Nₚ’ and ‘N_{q}’ are independently selected from any nucleotide from nucleotide 313 (A313) to nucleotide 411 (U411) of SEQ ID NO: 12. In some embodiments, the group I intron is an Anabaena sp. group I intron comprising the nucleotide sequence of SEQ ID NO: 49, ‘Nₚ’ and ‘N_{q}’ are independently selected from any nucleotide from nucleotide 212 (C212) to nucleotide 243 (G243) of SEQ ID NO: 49.

‘Nₚ’ may be located at any position upstream of ‘N_{q}’ in the group I intron. In some embodiments, ‘Nₚ’ is located immediately upstream of or adjacent to ‘N_{q}’ in the group I intron. In some embodiments, ‘Nₚ’ is located immediately upstream of ‘N_{q}’ in the group I intron. In some other embodiments, ‘Nₚ’ is located several nucleotides (for example, 2, 3, 4, 5, 6, 7, 8, 9, 10, 11, 12, 13, 14, 15, 16, 17, 18, 19, 20 or more nucleotides) upstream of ‘N_{q}’ in the group I intron.

In embodiments wherein the group I intron does not have a P9.2 duplex, ‘Nₚ’ can be the 3′ end nucleotide of the 5′ half of P9.0 duplex of the group I intron, and ‘N_{q}’ can be the 5′ end nucleotide of the 3′ half of P9.0 duplex of the group I intron. For example, for a Pneumocystis carinii group I intron comprising the nucleotide sequence of SEQ ID NO: 32, ‘Nₚ’ and ‘N_{q}’ can be nucleotide 316 (U316) and nucleotide 342 (G342) of SEQ ID NO: 32, respectively. For example, for an Anabaena sp. group I intron comprising the nucleotide sequence of SEQ ID NO: 49, ‘Nₚ’ and ‘N_{q}’ can be nucleotide 212 (C212) and nucleotide 243 (G243) of SEQ ID NO: 49, respectively.

In some embodiments, ‘Nₚ’ and ‘N_{q}’ can be independently selected from any nucleotide from the 3′ end nucleotide of the 5′ half to the 5′ end nucleotide of the 3′ half of a duplex of the group I intron, wherein the duplex is not a P9.0 duplex. For example, the duplex can be a P9a, P9b, P9.1, P9.1a or P9.2 duplex. In a preferable embodiment, the duplex is a P9.2 duplex.

In preferable embodiments, ‘Nₚ’ and ‘N_{q}’ are located within the region connecting the 5′ half and 3′ half of a duplex, wherein the duplex is not a P9.0 duplex. For example, ‘Nₚ’ and ‘N_{q}’ can be located within the apical loop of a P9a/9b, P9.1, P9.1a or P9.2 duplex. In an embodiment, the group I intron is a Pneumocystis carinii group I intron comprising the nucleotide sequence of SEQ ID NO: 32, ‘Nₚ’ and ‘N_{q}’ are independently selected from nucleotide 325 (G325) to nucleotide 328 (A328) of SEQ ID NO: 32. In another embodiment, the group I intron is a Tetrahymena thermophila group I intron comprising the nucleotide sequence of SEQ ID NO: 12, ‘Nₚ’ and ‘N_{q}’ are independently selected from any nucleotide from nucleotide 383 (G383) to nucleotide 386 (A386) of SEQ ID NO: 12. In yet another embodiment, the group I intron is an Anabaena sp. group I intron comprising the nucleotide sequence of SEQ ID NO: 49, ‘Nₚ’ and ‘N_{q}’ are independently selected from any nucleotide from nucleotide 219 (A219) to nucleotide A (A222) of SEQ ID NO: 49; or ‘Nₚ’ and ‘No’ are independently selected from any nucleotide from nucleotide 232 (G232) to nucleotide A (A235) of SEQ ID NO: 49.

In yet another embodiment, ‘Nₚ’ is the 3′ end nucleotide of the 5′ half of a duplex and ‘N_{q}’ is the 5′ end nucleotide of the 3′ half of a duplex, wherein the duplex is not a P9.0 duplex. For example, for a Pneumocystis carinii group I intron comprising the nucleotide sequence of SEQ ID NO: 32, ‘Nₚ’ and ‘N_{q}’ can be nucleotide 324 (C324) and nucleotide 329 (G329) of SEQ ID NO: 32, respectively. For example, for a Tetrahymena thermophila group I intron comprising the nucleotide sequence of SEQ ID NO: 12, ‘Nₚ’ and ‘N_{q}’ can be nucleotide 375 (U375) and nucleotide 394 (G394) of SEQ ID NO: 12, respectively; or ‘Nₚ’ and ‘N_{q}’ can be nucleotide 382 (C382) and nucleotide 387 (G387) of SEQ ID NO: 12, respectively. For example, for an Anabaena sp. group I intron comprising the nucleotide sequence of SEQ ID NO: 49, ‘Nₚ’ and ‘N_{q}’ can be nucleotide 218 (C218) and nucleotide 223 (G223) of SEQ ID NO: 49, respectively; or ‘Nₚ’ and ‘N_{q}’ can be nucleotide 231 (C231) and nucleotide 236 (G236) of SEQ ID NO: 49, respectively.

The IGS end of a group I intron can be readily identified by those skilled in the art in view of the present disclosure and the prior art. The second nucleotide sequence (corresponding to Ribozyme core-R2) may comprise a nucleotide sequence lacking the IGS of the group I intron. For example, the second nucleotide sequence may comprise a nucleotide sequence starting from the nucleotide immediately downstream of the 3′ end nucleotide of the IGS of a group I intron.

In some embodiments, the group I intron is a Pneumocystis carinii group I intron comprising the nucleotide sequence of SEQ ID NO: 32. In an embodiment, the second nucleotide sequence comprises a nucleotide sequence starting from nucleotide 18 (G18) to nucleotide 316 (U316) of SEQ ID NO: 32, and the first nucleotide sequence may comprise a nucleotide sequence starting from any nucleotide selected from nucleotide 317 (C317) to nucleotide 342 (G342) to the 3′ end of SEQ ID NO: 32. In another embodiment, the second nucleotide sequence comprises a nucleotide sequence starting from nucleotide 18 (G18) to any nucleotide selected from nucleotide 316 (U316) to nucleotide 341 (U341) of SEQ ID NO: 32, and the first nucleotide sequence may comprise a nucleotide sequence starting from nucleotide 342 (G342) to the 3′ end of SEQ ID NO: 32.

In some embodiments, the group I intron is a Tetrahymena thermophila group I intron comprising the nucleotide sequence of SEQ ID NO: 12. In an embodiment, the second nucleotide sequence may comprise a nucleotide sequence starting from nucleotide 27 (A27) to nucleotide 313 (A313) of SEQ ID NO: 12, and the first nucleotide sequence may comprise a nucleotide sequence starting from any nucleotide selected from nucleotide 314 (C314) to nucleotide 411 (U411) to the 3′ end of SEQ ID NO: 12. In another embodiment, the second nucleotide sequence may comprise a nucleotide sequence starting from nucleotide 27 (A27) to any nucleotide selected from nucleotide 313 (A313) to nucleotide 410 (C410) of SEQ ID NO: 12, and the first nucleotide sequence may comprise a nucleotide sequence starting from nucleotide 411 (U411) to the 3′ end of SEQ ID NO: 12.

In some embodiments, the group I intron is an Anabaena sp. group I intron comprising the nucleotide sequence of SEQ ID NO: 49. In an embodiment, the second nucleotide sequence may comprise a nucleotide sequence starting from nucleotide 12 (C12) to nucleotide 212 (C212) of SEQ ID NO: 49, and the first nucleotide sequence may comprise a nucleotide sequence starting from any nucleotide selected from nucleotide 213 (A213) to nucleotide 243 (G243) to the 3′ end of SEQ ID NO: 49. In another embodiment, the second nucleotide sequence may comprise a nucleotide sequence starting from nucleotide 12 (C12) to any nucleotide selected from nucleotide 212 (C212) to nucleotide 242 (A242) of SEQ ID NO: 49, and the first nucleotide sequence may comprise a nucleotide sequence starting from nucleotide 243 (G243) to the 3′ end of SEQ ID NO: 49.

In some embodiments, the RNA construct further comprises a 5′ homology arm sequence located upstream of the first nucleotide sequence and a 3′ homology arm sequence located downstream of the second nucleotide sequence, wherein the 5′ and 3′ homology arm sequences are as described above.

In some embodiments, the 5′ homology arm sequence comprises the nucleotide sequence of SEQ ID NO: 13, and the 3′ homology arm sequence comprises the nucleotide sequence of SEQ ID NO: 14. In some embodiments, the 5′ homology arm sequence comprises the nucleotide sequence of SEQ ID NO: 15 and the 3′ homology arm sequence comprises the nucleotide sequence of SEQ ID NO: 16. In some embodiments, the 5′ homology arm sequence comprises the nucleotide sequence of SEQ ID NO: 37 and the 3′ homology arm sequence comprises the nucleotide sequence of SEQ ID NO: 38. In some embodiments, the 5′ homology arm sequence comprises the nucleotide sequence of SEQ ID NO: 52 and the 3′ homology arm sequence comprises the nucleotide sequence of SEQ ID NO: 53.

However, the present inventor unexpectedly discovered that an RNA construct having a pair of homology arm sequences located at opposite ends of the RNA construct may achieve a high circularization efficiency comparable to an RNA construct counterpart preserving the native 3′ end sequence of a group I intron. That is, according to some embodiments of the present application, a 3′ end portion of the group I intron (e.g., a sequence from the 5′ half of the P9.0 duplex to the ωG) may be entirely replaced by a pair of homology arm sequences that are placed upstream of the GOI and downstream of the ribozyme core sequence, respectively, without affecting the circularization efficiency. Using homologous arm sequences to replace the natural partial sequences of a group I intron offers several advantages, including design simplicity and flexibility. When replacing the 3′ end portion of the group I intron (e.g., a sequence from the 5′ half of the P9.0 duplex to the ωG) with a pair of homologous arms, there is no need to add 5′ and 3′ spacers in the GOI region to ensure proper folding of the intron fragments at both ends. Furthermore, changes in the internal GOI sequence do not affect the circularization efficiency or interfere with the structure of the intron fragments at both ends. From a purification standpoint, the increased length difference between the 5′ and 3′ fragments generated after the splicing reaction facilitates their separation and purification. In summary, using homologous arm sequences for replacement of a 3′ end portion of a group I intron simplifies design, maintains structural integrity, and enhances purification efficiency.

The RNA construct may further comprises additional nucleotide sequences, for example, a nucleotide sequence useful for replication, transcription, translation and/or purification of the RNA construct, for example, inserted between two elements of the RNA construct as a spacer, or extending at the 5′ and/or 3′ ends of the RNA construct, as long as the self-splicing activity is maintained. Such nucleotide sequences may be conventionally selected by those skilled in the art as needed. In some embodiments, a spacer may be inserted between the 5′ homology arm and the first pairing sequence and/or between the 3′ homology arm and the second pairing sequence. In some embodiments, a spacer may be inserted between the 5′ homology arm and the first nucleotide sequence and/or between the 3′ homology arm and the second nucleotide sequence. In some embodiments, the 3′ end of the RNA construct can be extended with a sequence that will not pair to form a stable secondary structure such as a stem (referred to as “Tail element” in the present disclosure). Such sequences may include but are not limited to a polyadenine (polyA) and polyadenine/cytosine (polyAC) sequence of, for example, 10-200, 20-180, 30-150, 40-120, 50-100 nucleotides in length. In some embodiments, the RNA construct further comprises a polyA sequence at its 3′ end. The polyA sequence may comprise 10 to 150, preferably more than 20 and less than 100, and more preferably 40 to 70 consecutive adenines. This design can facilitate RNase R digestion of the precursor and can also increase the precursor's length difference versus the circRNA in favor of detection and purification (e.g., FIGS. 2 and 3).

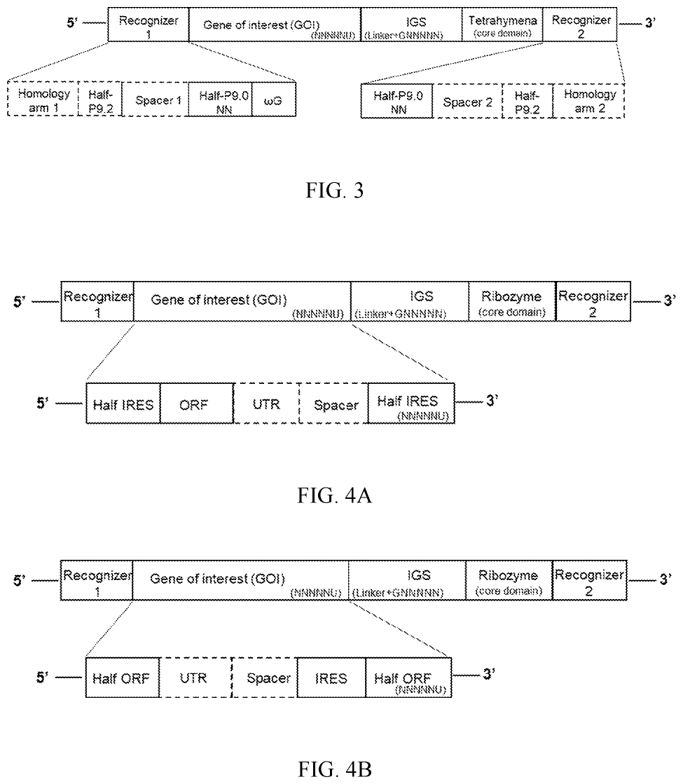

The nucleotide sequence of interest (GOI) can include but is not limited to the structure elements shown in FIG. 4. The nucleotide sequence of interest comprises a target site at its 3′ end. Preferably, the target site sequence is unique in the RNA construct. Base pairing between the target site and the IGS results in splicing at the 3′ end of the target site. After circularization, the 3′ end and 5′ end nucleotides of the nucleotide sequence of interest are connected to define the backsplicing site.

In one aspect, the present application provides an RNA construct that may achieve circularization of the nucleotide sequence of interest without inclusion of an exogenous exon fragment, for example, by mimicking the formation of a P1 duplex (P1 duplex mimic). Accordingly, advantageous effects of the present invention may at least include simplicity in design, a broad target sequence compatibility and/or a lower immunogenicity in a host while maintaining a high circularization efficiency. In some embodiments, the circular RNA does not comprise an exogenous exon fragment. For example, both the 3′ and the 5′ ends of the GOI do not comprise a natural exon fragment flanking the group I intron from which the ribozyme core sequence is derived. In some embodiments, the ribozyme core sequence is derived from a

Tetrahymena sp. group I intron. In some embodiments, the ribozyme core sequence is derived from a Tetrahymena thermophila group I intron comprising the nucleotide sequence of SEQ ID NO: 12. In some embodiments, the ribozyme core sequence comprises or consists of the nucleotide sequence of SEQ ID NO: 17 or a nucleotide sequence having at least 95% sequence identity thereto. In some embodiments, the ribozyme core sequence is derived from a Pneumocystis sp. group I intron. In some embodiments, the ribozyme core sequence is derived from a Pneumocystis sp. group I intron comprising a nucleotide sequence selected from SEQ ID NOs: 32-36. In some embodiments, the ribozyme core sequence comprises or consists of the nucleotide sequence of SEQ ID NO: 19 or a nucleotide sequence having at least 95% sequence identity thereto.

However, the present inventor unexpectedly discovered that in the cis-splicing system of the present application, for a ribozyme core sequence derived from, for example, an Anabaena sp. group I intron, a natural exon fragment flanking the group I intron may be desirable for a high circularization efficiency. In such cases, optimizing the 3′ end and/or 5′ end sequence of the GOI may be desirable to avoid the introduction of an exogenous exon sequence. This may be achieved by designing the backsplicing site in a non-coding region or codon optimization of a region in the nucleotide sequence to be circularized that is substantially homologous to an exon-exon junction fragment. In some embodiments, a 5′ end portion of the GOI (that is, a sequence that is downstream and adjacent to the ωN) may be designed to include a sequence that is substantially homologous, for example, at least 80%, 85%, 90%, 95%, 99% or 100% identical to a 5′ end portion of the 3′ exon (downstream exon) of the group I intron. In some embodiments, a 3′ end portion of the GOI (that is, a part of or the entire sequence of the target site and optionally its upstream sequence) may be designed to include a sequence that is substantially homologous, for example, at least 80%, 85%, 90%, 95%, 99% or 100% identical to a 3′ end portion of the 5′ exon (upstream exon) of the group I intron. In certain embodiments, the structure formed by the 5′ and 3′ termini of the GOI resembles the exon sequence structure found on both sides of the natural group I intron, where the 5′ and 3′ termini of the GOI can form an internal duplex. This structure may be introduced independently or integrated with the homologous sequences in the GOI. See for example, Chu-Xiao Liu et al., 2022, Molecular Cell, 82 (2): 420-434, for further description.

The present inventor further unexpectedly discovered that for a ribozyme core sequence derived from, for example, a Tetrahymena sp. group I intron or a Pneumocystis sp. group I intron, a high circularization efficiency may be achieved without the incorporation of a natural exon fragment. Accordingly, in some embodiments of the present application, a ribozyme core sequence derived from a Tetrahymena sp. group I intron or a Pneumocystis sp. group I intron as described herein may be preferable.

The backsplicing site can theoretically be set at any matching position of a nucleotide sequence to be circularized. In some embodiments, the backsplicing site can be designed inside the IRES (e.g., a sequence of ‘nnnnnu’ or ‘nnnnnc’ inside the IRES can be selected as the target site sequence). After circularization, IRES fragments at both ends of GOI can be reconnected to form a complete IRES sequence, as shown in FIG. 4A. In some embodiments, the backsplicing site can be designed inside the ORF (e.g., a sequence of ‘nnnnnu’ or ‘nnnnnc’ inside the ORF can be selected as the target site sequence). After circularization, ORF fragments at both ends of GOI can be reconnected to form a complete ORF sequence, as shown in FIG. 4B. UTRs can be native 3′ UTR sequences or modified noncoding sequences. Spacers can be native 5′ UTR sequences or other noncoding sequences, including but not limited to aptamers, polyACs, translation-enhancing sequences, purification-related sequences, etc. The IGS region comprises an internal guide sequence (IGS). The 5′ end nucleotide of the IGS and the 3′ end nucleotide of the target site form a non-Watson-Crick base pair to define the 5′ splice site. Preferably, the non-Watson-Crick base pair is a wobble base pair. In some embodiments, the wobble base pair formed between the 5′ end nucleotide of the IGS and the 3′ end nucleotide of the target site is guanine-uracil (G-u), wherein ‘G’ is the 5′ end nucleotide of the IGS and ‘u’ is the 3′ end nucleotide of the target site. In some embodiments, the wobble base pair is adenine-cytosine (A-c), wherein ‘A’ is the 5′ end nucleotide of the IGS and ‘c’ is the 3′ end nucleotide of the target site. In some embodiments, the non-Watson-Crick base pair formed between the 5′ end nucleotide of the IGS and the 3′ end nucleotide of the target site is guanine-adenine (G-a), wherein ‘G’ is the 5′ end nucleotide of the IGS and ‘a’ is the 3′ end nucleotide of the target site. In some embodiments, the ribozyme core sequence is derived from a Pneumocystis carinii or Tetrahymena sp. group I intron, the wobble base pair is adenine-cytosine (A-c).

In some embodiments, the IGS and the target site form a P1 duplex mimic. The P1 duplex mimic may comprise a Watson-Crick base pair, a wobble base pair or a combination thereof. The P1 duplex mimic may comprise at least on base pair. For example, the P1 duplex mimic may comprise 1-20 base pairs, e.g., 1, 2, 3, 4, 5, 6, 7, 8, 9 or 10 base pairs. Preferably, the P1 duplex mimic comprises a substantially identical number of base pairs to that of the P1 duplex of the group I intron from which the ribozyme core sequence is derived. Those skilled in the art would be able to determine essential features for a P1 duplex mimic in view of the present disclosure and the prior art.

In some embodiments, the IGS has the structure of 5′-X(N)ₘ-3′, and the target site has the structure of 5′-(n)ₘx-3′, wherein
‘X’ and ‘x’ are the nucleotides that form the non-Watson-Crick base pair,
each ‘N’ and ‘n’ is a nucleotide independently selected from adenine (A), cytosine (C), guanine (G), uracil (U), pseudouridine (Ψ), 1-methylpseudouridine (1 mΨ), 5-methoxyuridine (5moU), 2-thiouridine, 4-thiouridine, 5-methylcytidine, and N6-methyladenosine, e.g., wherein each ‘N’ and ‘n’ is a nucleotide independently selected from adenine (A), cytosine (C), guanine (G), and uracil (U); and
m is an integer of 2-8.

In some embodiments, m is an integer of 3-6. In some embodiments, m is an integer of 4-5. In a particular embodiment, m is 5.

In some embodiments, the base pairs formed between 5′-(N)ₘ-3′ and 5′-(n)ₘ-3′ comprise a Watson-Crick base pair, a wobble base pair or a combination thereof. In some embodiments, 5′-(N)ₘ-3′ and 5′-(n)ₘ-3′ are reverse complementary.

In some embodiments, the IGS comprises a sequence ‘GNNNNN’ and the target site comprises a sequence ‘nnnnnu’. In some embodiments, the IGS comprises a sequence ‘ANNNNN’ and the target site comprises a sequence ‘nnnnnc’. In some embodiments, ‘NNNNN’ and ‘nnnnn’ are reverse complementary.

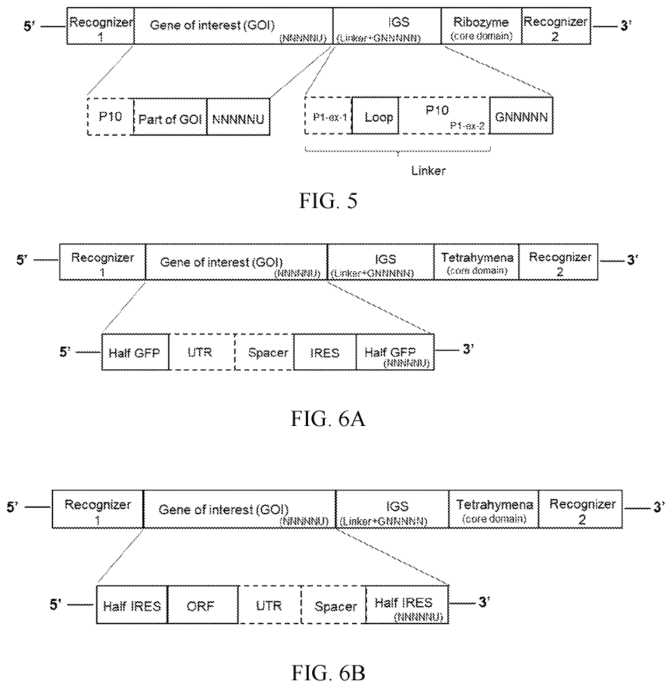

In some embodiments, the RNA construct may further comprise a linker sequence located between the target site and IGS. The linker sequence can include but are not limited to the sequence elements as shown in FIG. 5. The linker sequence may comprise 1-50 nucleotides, for example, 2-45, 3-40, 4-30, 5-25, 6-20, 7-15 or 8-10 nucleotides.

In some embodiments, the linker sequence comprises an unpaired sequence. The unpaired sequence may form a loop structure between the target site and the IGS. In some embodiments, the linker sequence comprises an unpaired sequence, wherein the target site, the linker sequence and the IGS form a stem-loop structure. In some embodiments, the stem portion of the stem-loop structure may comprise at least two base pairs, for example, 2-20 base pairs, for example, 2, 3, 4, 5, 6, 7, 8, 9, 10, 11, 12, 13, 14 or 15 base pairs or more. In some embodiments, the loop portion of the stem-loop structure may comprise at least 3 nucleotides, for example, 3-50 nucleotides, for example, 3, 4, 5, 6, 7, 8, 9, 10, 11, 12, 13, 14, 15, 16, 17, 18, 19, 20, 25-50, 30-45 or 35-40 nucleotides. The stem-loop structure may also have on either side of the stem one or more bulges (mismatches). The unpaired sequence may comprise at least 3 nucleotides, for example, 3-50 nucleotides, for example, 3, 4, 5, 6, 7, 8, 9, 10, 11, 12, 13, 14, 15, 16, 17, 18, 19, 20, 25-50, 30-45 or 35-40 nucleotides. Examples of an unpaired sequence may be a polyA or polyU sequence.

The IGS (e.g., ‘GNNNNN’ or ‘ANNNNN’) can extend 1 to 3 nucleotides at the 5′ end and form a P1 extension (P1-ex) mimic with 1 to 3 nucleotides adjacent to the target site (e.g., ‘nnnnnu’ or ‘nnnnnc’, respectively) at the 3′ end of GOI. In some embodiments, the linker sequence comprises, from 5′ to 3′ end, a third pairing sequence, a loop sequence, and a fourth pairing sequence, wherein the third and fourth pairing sequences form a P1 extension mimic. The P1 extension mimic may comprise a Watson-Crick base pair, a wobble base pair or a combination thereof. The P1 extension mimic may comprise 1, 2, 3, 4, 5, 6, or more base pairs, preferably 1-3 base pairs. In some embodiments, the P1 extension mimic comprises 1-3 reverse complementary base pairs. In some embodiments, the third pairing sequence comprises a sequence of 1-3 contiguous nucleotides, which is reverse complementary to a sequence of the same number of contiguous nucleotides in the fourth pairing sequence to form a P1 extension mimic.

The RNA construct may further comprise a fifth pairing sequence which can pair with a sequence in the GOI which is adjacent to the ωN (e.g., ωG in some embodiments) to simulate the formation of a P10 duplex (also referred to as a “P10 duplex mimic”). In some embodiments, the linker sequence comprises a fifth pairing sequence which can pair with a sixth pairing sequence in the 5′ region of the nucleotide sequence of interest to form a P10 duplex mimic. The P10 duplex mimic may comprise a Watson-Crick base pair, a wobble base pair or a combination thereof. The P10 duplex mimic may comprise at least two consecutive base pairs, for example, 3-10 base pairs, preferably 3, 4, 5, 6, 7, or 8 base pairs. In some embodiments, the P10 duplex mimic comprises 3-10 reverse complementary base pairs. In some embodiments, the fifth pairing sequence comprises a sequence of 3-10 contiguouse nucleotides, which is reverse complementary to a sequence of the same number of contiguous nucleotides in the sixth pairing sequence to form a P10 duplex mimic.

The sixth pairing sequence may be located adjacent to the 5′ end of the nucleotide sequence of interest, for example, starting from the nucleotide immediately downstream of the ωN (i.e., starting from the nucleotide 1 of the nucleotide sequence of interest (the nucleotide at the ωN+1 position in the RNA construct) or starting from a few nucleotides downstream of the ωN (for example, starting from the nucleotide 2 or 3 of the nucleotide sequence of interest (the nucleotide at the ωN+2 or N+3 position in the RNA construct). In some embodiments, the sixth pairing sequence starts from a nucleotide at a ωN+r position in the RNA construct, wherein r is an integer greater or equal to 1, for example r is an integer of 1-50, 10-40, 20-30, for example, r is 1, 2, 3, 4, 5, 6, 7, 8, 9, 10, 11, 12, 13, 14, 15, 16, 17, 18, 19 or 20. In some preferable embodiments, the sixth pairing sequence starts from the nucleotide at the ωN+1 position in the RNA construct. In some embodiments, ωN is guanine.

In some embodiments, the RNA construct comprises sequences for a P1 extension mimic but not a P10 duplex mimic. In some embodiments, the linker sequence comprises, from 5′ to 3′ end, a third pairing sequence, a loop sequence, and a fourth pairing sequence, wherein the third and fourth pairing sequences form a P1 extension mimic, and part or the entire of a 3′ end portion of the linker sequence does not pair with a sequence in the 5′ region of the GOI.

In some embodiments, the RNA construct comprises sequences for a P10 duplex mimic but not a P1 extension mimic. In some embodiments, the linker sequence comprises a loop sequence, and a 3′ end portion of the loop sequence constitute a fifth pairing sequence which can pair with a sixth pairing sequence in the 5′ region of the GOI to form a P10 duplex mimic.

In some embodiments, the RNA construct comprises sequences for a P1 extension mimic and a P10 duplex mimic. In some embodiments, the fifth pairing sequence for the P10 duplex mimic and the fourth pairing sequence for the P1 extension mimic partially overlap. In some embodiments, the linker sequence comprises, from 5′ to 3′ end, a third pairing sequence, a loop sequence and a fourth pairing sequence, wherein the third and fourth pairing sequences form a P1 extension mimic, and a 3′ end portion of the loop sequence and a 5′ end portion or the entire of the fourth pairing sequence constitute a fifth pairing sequence which can pair with a sixth pairing sequence in the 5′ region of the GOI to form a P10 duplex mimic.

In some embodiments, the RNA construct has the structure of the following:
5′-5′ homology arm sequence-(Nₓ)ₕ(N_{y})ₜG-GOI-linker sequence-IGS-ribozyme core sequence-(nₓ)ₕ-3′ homology arm sequence-3′
wherein
‘Nₓ’, ‘nₓ’ and ‘N_{y}’ each is independently any naturally occurring or modified nucleotide,
‘(Nₓ)ₕ’ and ‘(nₓ)ₕ’ are reverse complementary,
h is an integer of 2-6,
t is an integer of 0-20,
the 5′ and 3′ homology arm sequences are substantially complementary, and
the ribozyme core sequence, the linker sequence, and the target site and the IGS are as defined above.

In some embodiments, t is an integer of 0-10.

In some embodiments, t is 0, the RNA construct has the structure of the following:
5′-5′ homology arm sequence-(Nₓ)ₕG-GOI-linker sequence-IGS-ribozyme core sequence-(nₓ)ₕ-3′ homology arm sequence-3′.

In some embodiments, the IGS comprises a sequence ‘GNNNNN’ and the target site comprises a sequence ‘nnnnnu’. In some embodiments, the IGS comprises a sequence ‘ANNNNN’ and the target site comprises a sequence ‘nnnnnc’. In some embodiments, ‘NNNNN’ and ‘nnnnn’ are reverse complementary.

The nucleotide sequence to be circularized can be split into a 5′ fragment ended with the selected target site and a 3′ fragment comprising the remaining sequence. The nucleotide sequence of interest may be formed by placing the 3′ fragment at the 5′ region and the 5′ fragment at the 3′ region of the GOI. In some embodiments, the circular RNA is formed by connecting the nucleotide immediately downstream of the ωN (i.e., the nucleotide at the ωN+1 position in the RNA construct) and the 3′ end nucleotide of the target site through the self-splicing of the RNA construct. Accordingly, in some embodiments, the circular RNA may substantially consist of the nucleotide sequence of interest. In some embodiments, the circular RNA is formed by connecting the nucleotide at ωN+1 position and the 3′ end nucleotide of the target site in the RNA construct. In some embodiments, ωN is guanine.

In some embodiments, the circular RNA comprises a noncoding sequence having a biological activity. Examples of a noncoding sequence having a biological activity include, but are not limited to, a micro RNA and a long non-coding (lnc) RNA. In some embodiments, the circular RNA comprises a protein-coding sequence. The protein-coding sequence may encode any protein, for example, a protein for therapeutic or diagnostic use. In some embodiments, the protein-coding sequence encodes an antibody.

When the circular RNA comprises a protein-coding sequence, the circular RNA may further comprise sequences necessary for translation, e.g., an internal ribosomal entry site (IRES) sequence upstream of the protein-coding sequence. In some embodiments, the IRES sequence is intact within the nucleotide sequence of interest. In some embodiments, the IRES sequence is split to the 5′ and 3′ ends of the nucleotide sequence of interest and connected after circularization (e.g., FIG. 4A). In some embodiments, the circular RNA comprises an IRES sequence operably linked to a protein-coding sequence. As used herein, the phrase “operably linked” means that the IRES sequence is positioned upstream of the protein-coding sequence such that the protein-coding sequence can be translated into a protein in vivo (inside eukaryotic cells, e.g., human cells) and/or in vitro. The IRES sequence may be any IRES sequence known in the art. The IRES sequence may be naturally occurring or recombinant, e.g., obtained by truncating or mutating a naturally occurring IRES sequence. In some embodiments, the IRES sequence is selected from an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, human rhinovirus B,Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Human enterovirus B, Equine rhinitis virus, Ectropis obliqua picoma-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n-myc, Mouse Gtx, Human p27kip1, Human PDGF2/c-sis, Human p53, Human Pim-1, Mouse Rbm3, Drosophila reaper, Canine Scamper, Drosophila Ubx, Salivirus, Cosavirus, Parechovirus, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2). In certain embodiments, the IRES sequence is a IRES sequence of Coxsackievirus B3 (CVB3). In certain embodiments, the IRES sequence is a IRES sequence of Human rhinovirus B.

The nucleotide sequence of interest may comprise at least two protein-coding regions such that at least two different proteins can be expressed from the circular RNA. For example, a 2A or 2A-like sequence may be included between two protein-coding sequences to mediate co-translation of two proteins (also referred to as “Stop-Carry On” or “StopGo” translation). See, for example, de Lima JGS, Lanza DCF. 2A and 2A-like Sequences: Distribution in Different Virus Species and Applications in Biotechnology. Viruses. 2021 Oct. 26; 13 (11): 2160. Alternatively, two or more different IRES sequences may be used to drive the expression of two or more different protein-coding regions.

The RNA construct may comprise unmodified or modified nucleotides. In some embodiments, the RNA construct does not comprise uridine, but comprises nucleosides selected from pseudouridine (Ψ), 1-methylpseudouridine (1 mΨ), 5-methoxyuridine (5 moU), 2-thiouridine, or 4-thiouridine in place of uridine. In some embodiments, the RNA construct comprises 10%-100%, for example, 10%-90%, 20-80%, 30%-70%, 40-60%, or 50%-60% modified uridine in place of uridine, wherein the modified uridine is selected from pseudouridine (Ψ), 1-methylpseudouridine (1 mΨ), 5-methoxyuridine (5 moU), 2-thiouridine, or 4-thiouridine.

The circular RNA may be of any length. For example, the circular RNA may comprise about 200-10,000 nucleotides (e.g., about 300, about 400, about 500, about 600, about 700, about 800, about 900, about 1,000, about 2,000, about 3,000, about 4,000, about 5,000, about 6,000, about 7,000, about 8,000, or about 9,000 nucleotides, or a range defined by any two of the foregoing values). In some embodiments, the circular RNA comprises about 500-6,000 nucleotides (e.g., about 550, about 650, about 750, about 850, about 950, about 1,100, about 1,200, about 1,300, about 1,400, about 1,500, about 1,600, about 1,700, about 1,800, about 1,900, about 2,100, about 2,200, about 2,300, about 2,400, about 2,500, about 2,600, about 2,700, about 2,800, about 2,900, about 3,100, about 3,300, about 3,500, about 3,700, about 3,800, about 3,900, about 4,100, about 4,300, about 4,500, about 4,700, about 4,900, about 5,100, about 5,300, about 5,500, about 5,700, or about 5,900 nucleotides, or a range defined by any two of the foregoing values).

The disclosure provides, in a first aspect, an RNA construct (Construct 1) comprising,
a first recognizersequence (R1) comprising a first pairing sequence;
a nucleotide sequence of interest (GOI) comprising a target site at its 3′ end;
a ribozyme core sequence operably linked to an internal guide sequence (IGS), wherein the ribozyme core sequence encodes a ribozyme core having the catalytic activity of a group I intron ribozyme; and
a second recognizer sequence (R2) comprising a second pairing sequence substantially complementary to the first pairing sequence;
wherein
the 5′ end nucleotide of the IGS and the 3′ end nucleotide of the target site form a non-Watson-Crick base pair to define a 5′ splice site;
R1 and R2 are positioned at opposite ends of the RNA construct, such that hybridization of the first and second pairing sequences results in formation of a duplex-containing structure to define a 3′ splice site;
the GOI is positioned 5′ to the ribozyme core sequence and IGS; and
the RNA construct is capable of generating a circular RNA comprising the nucleotide sequence of interest through the catalytic activity of the ribozyme core.

For example, the present disclosure provides:
1.1. Construct 1, comprising, from 5′ end to 3′ end,
   R1 comprising a first pairing sequence and a 3′ end nucleotide ‘N’ (ωN);
   GOI comprising a target site at its 3′ end,
   IGS;
   Ribozyme core sequence; and
   R2 comprising a second pairing sequence;
   wherein
   ωN is any naturally occurring or modified nucleotide; and
   the first pairing sequence and the second pairing sequence are substantially complementary to form a duplex-containing structure upstream of the ωN to define the 3′ splice site.
1.2. The RNA construct of 1.1, wherein ωN is guanine (ωG).
1.3. Any forgoing RNA construct, wherein the ribozyme core sequence comprises a nucleotide sequence encoding the scaffold domain and catalytic domain of a group I intron; preferably, the ribozyme core sequence comprises or consists of the sequence from the IGS end to the sequence before the P9.0 duplex of a group I intron.
1.4. Any foregoing RNA construct, wherein the ribozyme core sequence is derived from a group IC1 (e.g., from Tetrahymena sp. (e.g., T. thermophile, T. cosmopolitanis, T. hyperangularis, T. malaccensis or T. pigmentosa) or Pneumocystis sp. (e.g., Pneumocystis carinii), IC2, IC3 (e.g., from Anabaena sp. PCC7120 or Azoarcus sp. BH72), or IA2 (e.g., from Bacteriophage Twort) intron.
1.5. Any foregoing RNA construct, wherein the ribozyme core sequence is derived from a Pneumocystis carinii group I intron; for example, a Pneumocystis sp. group I intron comprising a nucleotide sequence selected from SEQ ID NOs: 32-36; preferably, the ribozyme core sequence is derived from a Pneumocystis carinii group I intron comprising the nucleotide sequence of SEQ ID NO: 32, for example, the ribozyme core sequence comprises or consists of the nucleotide sequence of SEQ ID NO: 19 or a nucleotide sequence having at least 95% sequence identity thereto.
1.6. Construct 1 or any RNA construct of 1.1-1.3, wherein the ribozyme core sequence is derived from a Tetrahymena sp. group I intron; for example, a Tetrahymena thermophila group I intron comprising the nucleotide sequence of SEQ ID NO: 12, for example, the ribozyme core sequence comprises or consists of the nucleotide sequence of SEQ ID NO: 17 or a nucleotide sequence having at least 95% sequence identity thereto.
1.7 Construct 1 or any RNA construct of 1.1-1.3, wherein the ribozyme core sequence is derived from an Anabaena sp. group I intron; for example, the ribozyme core sequence comprises or consists of the nucleotide sequence of SEQ ID NO: 48 or a nucleotide sequence having at least 95% sequence identity thereto.
1.8. Any foregoing RNA construct, wherein the duplex-containing structure comprises one or more base pairs, consecutive or interrupted by one or more mismatches.
1.9. Any foregoing RNA construct, wherein the first and second pairing sequences each independently comprises 1-200 nucleotides; for example, the first pairing sequence comprises 2-20, 2-12, 4-10, 6, 7 or 8 nucleotides; and/or the second pairing sequence comprises 2-100, 5-80, 8-60, 10-50, 15, 20, 30, 40, 50, 60, 70, 80, 90 or 100, preferably 5-80 or 8-60 nucleotides.
1.10. Any foregoing RNA construct, wherein the first and second pairing sequences form at least two consecutive base pairs, preferably 2-6 consecutive base pairs, immediately upstream of the ωN.
1.11. Any foregoing RNA construct, wherein the first and second pairing sequences form a Watson-Crick base pair, a non-Watson-Crick base pair or a combination thereof; preferably, the non-Watson-Crick base pair is a wobble base pair, for example, a G-U wobble base pair; more preferably, the first and second pairing sequences form a base pair selected from A-U, G-C, G-A, A-A, U-U, A-C, G-U and a combination thereof.
1.12. Any foregoing RNA construct, wherein R1 further comprises a 5′ homology arm sequence located upstream of the first pairing sequence and R2 further comprises 3′ homology arm sequence located downstream of the second pairing sequence, and the 5′ and 3′ homology arm sequences are substantially reverse complementary; for example, the 5′ homology arm sequence comprises the nucleotide sequence of SEQ ID NO: 13, and the 3′ homology arm sequence comprises the nucleotide sequence of SEQ ID NO: 14; or the 5′ homology arm sequence comprises the nucleotide sequence of SEQ ID NO: 15, and the 3′ homology arm sequence comprises the nucleotide sequence of SEQ ID NO: 16; or the 5′ homology arm sequence comprises the nucleotide sequence of SEQ ID NO: 37, and the 3′ homology arm sequence comprises the nucleotide sequence of SEQ ID NO: 38; or the 5′ homology arm sequence comprises the nucleotide sequence of SEQ ID NO: 52 and the 3′ homology arm sequence comprises the nucleotide sequence of SEQ ID NO: 53.
1.13. An RNA construct comprising, from 5′ end to 3′ end,
   a first recognizer sequence (R1) comprising a nucleotide sequence ‘(Nₓ)ₛ(N_{y})ₜ(ωN)’ at its 3′ end;
   a nucleotide sequence of interest (GOI) comprising a target site at its 3′ end;
   an internal guide sequence (IGS);
   a ribozyme core sequence encoding a ribozyme core which has the catalytic activity of a group I intron ribozyme; and
   a second recognizer sequence (R2) comprising a nucleotide sequence ‘(nₓ)_{w}’;
   wherein
   the 5′ end nucleotide of the IGS and the 3′ end nucleotide of the target site form a non-Watson-Crick base pair to define a 5′ splice site;
   ωN, ‘Nₓ’, ‘nₓ’, and ‘N_{y}’ are each independently any naturally occurring or modified nucleotide;
   t is an integer of 0-20;
   s and w are each independently an integer of 1-200;
   ‘(Nₓ)ₛ’ and ‘(nₓ)_{w}’ are substantially complementary to form a duplex-containing structure upstream of the ωN to define a 3′ splice site; and
   the RNA construct is capable of generating a circular RNA comprising the nucleotide sequence of interest through the catalytic activity of the ribozyme core.
1.14. The RNA construct of 1.13, wherein ωN is guanine (ωG).
1.15. The RNA construct of 1.13 or 1.14, wherein the ribozyme core sequence is as defined in any one of 1.3-1.7.
1.16. The RNA construct of any one of 1.13-1.15, wherein t is an integer of 0-10.
1.17. The RNA construct of any one of 1.13-1.16, wherein t is 0 or 1.
1.18. The RNA construct of any one of 1.13-1.17, wherein each of s and w is an integer of h which is selected from 2-6, and ‘(Nₓ)ₕ’ and ‘(nₓ)ₕ’ are reverse complementary.
1.19. The RNA construct of 1.13, wherein R1 comprises a nucleotide sequence ‘N₂N₁G’ at its 3′ end; or t is 1, R1 comprises a nucleotide sequence ‘N₂N₁N_{y}G’ at its 3′ end; and R2 comprises a nucleotide sequence ‘n₁n₂’; wherein ‘N₁’, ‘n₁’, ‘N₂’, ‘n₂’ and ‘N_{y}’ are each independently any naturally occurring or modified nucleotide, preferably, ‘N_{y}’ is ‘G’, ‘U’ or ‘A’; wherein ‘N₁’ and ‘n₁’ form a first base pair, and ‘N₂’ and ‘n₂’ form a second base pair.
1.20. The RNA construct of any one of 1.13-1.19, wherein R1 further comprises a 5′ homology arm sequence located upstream of the ‘(Nₓ)ₛ(N_{y})ₜ(ωN)’, and R2 further comprises a 3′ homology arm sequence located downstream of the ‘(nₓ)_{w}’, wherein the 5′ and 3′ homology arm sequences are substantially complementary.
1.21. The RNA construct of 1.20, wherein the 5′ homology arm sequence comprises the nucleotide sequence of SEQ ID NO: 13, and the 3′ homology arm sequence comprises the nucleotide sequence of SEQ ID NO: 14; or the 5′ homology arm sequence comprises the nucleotide sequence of SEQ ID NO: 15, and the 3′ homology arm sequence comprises the nucleotide sequence of SEQ ID NO: 16; or the 5′ homology arm sequence comprises the nucleotide sequence of SEQ ID NO: 37, and the 3′ homology arm sequence comprises the nucleotide sequence of SEQ ID NO: 38; the 5′ homology arm sequence comprises the nucleotide sequence of SEQ ID NO: 52, and the 3′ homology arm sequence comprises the nucleotide sequence of SEQ ID NO: 53.

The present disclosure provides, in a second aspect, an RNA construct (Construct 2) comprising, from 5′ end to 3′ end,
a first nucleotide sequence comprising a sequence from a nucleotide ‘N_{q}’ to the 3′ end of a group I intron,
a nucleotide sequence of interest (GOI) comprising a target site at its 3′ end,
an internal guide sequence (IGS), and
a second nucleotide sequence comprising a sequence from the IGS end to a nucleotide ‘Nₚ’ of a group I intron;
wherein
the 5′ end nucleotide of the IGS and the 3′ end nucleotide of the target site form a non-Watson-Crick base pair to define a 5′ splice site;
‘Nₚ’ and ‘N_{q}’ are independently selected from any nucleotide from the 3′ end nucleotide of the 5′ half to the 5′ end nucleotide of the 3′ half of P9.0 duplex of the group I intron, and
‘Nₚ’ is located upstream of ‘N_{q}’ in the group I intron.

For example, the present disclosure provides:
1.22. Construct 2, wherein the group I intron is a group IC1 (e.g., from Tetrahymena sp. or Pneumocystis sp. (e.g., Pneumocystis carinii), IC2, IC3 (e.g., from Anabaena sp. PCC7120 or Azoarcus sp. BH72), or IA2 (e.g., from Bacteriophage Twort) intron; preferably, the group I intron is a Pneumocystis sp. group I intron comprising a nucleotide sequence selected from SEQ ID NOs: 32-36, or a Tetrahymena thermophila group I intron comprising the nucleotide sequence of SEQ ID NO: 12.
1.23. Construct 2 or the RNA construct of 1.22, wherein the group I intron is a Pneumocystis carinii group I intron comprising the nucleotide sequence of SEQ ID NO: 32, ‘Nₚ’ and ‘N_{q}’ are independently selected from any nucleotide from nucleotide 316 to nucleotide 342 of SEQ ID NO: 32; or the group I intron is a Tetrahymena thermophila group I intron comprising the nucleotide sequence of SEQ ID NO: 12, ‘Nₚ’ and ‘N_{q}’ are independently selected from any nucleotide from nucleotide 313 to nucleotide 411 of SEQ ID NO: 12; or the group I intron is an Anabaena sp. group I intron comprising the nucleotide sequence of SEQ ID NO:49, ‘Nₚ’ and ‘N_{q}’ are independently selected from any nucleotide from nucleotide 212 to nucleotide 243 of SEQ ID NO: 49.
1.24. Construct 2 or the RNA construct of 1.22, wherein ‘Nₚ’ and ‘N_{q}’ are independently selected from any nucleotide from the 3′ end nucleotide of the 5′ half to the 5′ end nucleotide of the 3′ half of a duplex of the group I intron, wherein the duplex is not a P9.0 duplex; for example, the duplex is a P9a/9b, P9.1, P9.1a or P9.2 duplex, preferably a P9.2 duplex.
1.25. Construct 2 or the RNA construct of 1.22, wherein ‘Nₚ’ and ‘N_{q}’ are located within the region connecting the 5′ half and 3′ half of the duplex; or ‘Nₚ’ is the 3′ end nucleotide of the 5′ half of the duplex and ‘N_{q}’ is the 5′ end nucleotide of the 3′ half of the duplex.
1.26. Construct 2 or the RNA construct of any one of 1.22-1.25, wherein the RNA construct further comprises a 5′ homology arm sequence located upstream of the first nucleotide sequence and a 3′ homology arm sequence located downstream of the second nucleotide sequence, wherein the 5′ and 3′ homology arm sequences are substantially complementary; for example, the 5′ homology arm sequence comprises the nucleotide sequence of SEQ ID NO: 13, and the 3′ homology arm sequence comprises the nucleotide sequence of SEQ ID NO: 14; or the 5′ homology arm sequence comprises the nucleotide sequence of SEQ ID NO: 15, and the 3′ homology arm sequence comprises the nucleotide sequence of SEQ ID NO: 16; or the 5′ homology arm sequence comprises the nucleotide sequence of SEQ ID NO: 37, and the 3′ homology arm sequence comprises the nucleotide sequence of SEQ ID NO: 38; or the 5′ homology arm sequence comprises the nucleotide sequence of SEQ ID NO: 52, and the 3′ homology arm sequence comprises the nucleotide sequence of SEQ ID NO: 53.

The present disclosure further provides:
1.27. Any foregoing RNA construct, wherein the non-Waton-Crick base pair formed between the 5′ end nucleotide of the IGS and the 3′ end nucleotide of the target site is
   (a) guanine-uracil (G-u), wherein ‘G’ is the 5′ end nucleotide of the IGS and ‘u’ is the 3′ end nucleotide of the target site; or
   (b) adenine-cytosine (A-c), wherein ‘A’ is the 5′ end nucleotide of the IGS and ‘c’ is the 3′ end nucleotide of the target site; or
   (c) guanine-adenine (G-a), wherein ‘G’ is the 5′ end nucleotide of the IGS and ‘a’ is the 3′ end nucleotide of the target site.
1.28. Any foregoing RNA construct, wherein the IGS and the target site form a P1 duplex mimic.
1.29. Any foregoing RNA construct, wherein
   the IGS has the structure of 5′-X(N)ₘ-3′
   the target site has the structure of 5′-(n)ₘx-3′
   ‘X’ and ‘x’ are the nucleotides that form the non-Watson-Crick base pair,
   each ‘N’ and ‘n’ is a nucleotide independently selected from adenine (A), cytosine (C), guanine (G), uracil (U), pseudouridine (Ψ), 1-methylpseudouridine (1 mΨ), 5-methoxyuridine (5 moU), 2-thiouridine, 4-thiouridine, 5-methylcytidine, and N6-methyladenosine, e.g., wherein each ‘N’ and ‘n’ is a nucleotide independently selected from adenine (A), cytosine (C), guanine (G), and uracil (U); and
   m is an integer of 2-8, preferably 3-6, most preferably 4-5;
   preferably, 5′-(N)ₘ-3′ and 5′-(n)ₘ-3′ are reverse complementary.
1.30. Any foregoing RNA construct, wherein
   the IGS comprises a sequence ‘GNNNNN’ and the target site comprises a sequence ‘nnnnnu’; or
   the IGS comprises a sequence ‘ANNNNN’ and the target site comprises a sequence ‘nnnnnc’;
   wherein ‘NNNNN’ and ‘nnnnn’ are reverse complementary.
1.31. Any foregoing RNA construct, wherein
   the RNA construct further comprises a linker sequence located between the target site and IGS.
1.32. The RNA construct of 1.31, wherein the linker sequence comprises an unpaired sequence, wherein the target site, the linker sequence and the IGS form a stem-loop structure.
1.33. The RNA construct of 1.31, wherein the linker sequence comprises, from 5′ end to 3′ end, a third pairing sequence, a loop sequence and a fourth pairing sequence, wherein the third and fourth pairing sequences form a P1 extension mimic; preferably, the P1 extension mimic comprises 1-3 reverse complementary base pairs.
1.34. The RNA construct of any one of 1.31-1.33, wherein the linker sequence comprises a fifth pairing sequence which can pair with a sixth pairing sequence in the 5′ region of the GOI to form a P10 duplex mimic; preferably, the P10 duplex mimic comprises 3-10 base pairs.
1.35. Any foregoing RNA construct, having the structure of the following:
   5′-5′ homology arm sequence-(Nₓ)ₕ(N_{y})ₜG-GOI-linker sequence-IGS-ribozyme core sequence-(nₓ)ₕ-3′ homology arm sequence-3′
   wherein
   ‘Nₓ’, ‘nₓ’ and ‘N_{y}’ each is independently any naturally occurring or modified nucleotide, ‘(Nₓ)ₕ’ and ‘(nₓ)ₕ’ are reverse complementary,
   h is an integer of 2-6,
   t is an integer of 0-20,
   the ribozyme core sequence is as defined in any one of 1.3-1.7,
   the linker sequence is as defined in any one of 1.32-1.34,
   the target site and the IGS are as defined in any one of 1.27-1.30, and
   the 5′ and 3′ homology arm sequences are substantially complementary.
1.36. The RNA construct of 1.35, wherein t is an integer of 0-10; preferably, t is 0.
1.37. The RNA construct of 1.35 or 1.36, wherein the IGS comprises a sequence ‘GNNNNN’ and the target site comprises a sequence ‘nnnnnu’ or the IGS comprises a sequence ‘ANNNNN’ and the target site comprises a sequence ‘nnnnnc’, and ‘NNNNN’ and ‘nnnnn’ are reverse complementary.
1.38. The RNA construct of 1.13, having the structure of the following:
   (a) 5′-SEQ ID NO: 21-GOI-linker sequence-IGS-SEQ ID NO: 17-SEQ ID NO: 20-3′;
   (b) 5′-SEQ ID NO: 23-GOI-linker sequence-IGS-SEQ ID NO: 17-SEQ ID NO: 22-3′;
   (c) 5′-SEQ ID NO: 25-GOI-linker sequence-IGS-SEQ ID NO: 17-SEQ ID NO: 24-3′; or
   (d) 5′-SEQ ID NO: 27-GOI-linker sequence-IGS-SEQ ID NO: 17-SEQ ID NO: 26-3′;
   (e) 5′-GUG-GOI-linker sequence-IGS-SEQ ID NO: 19-AU-3′;
   (f) 5′-ACG-GOI-linker sequence-IGS-SEQ ID NO: 19-GU-3′;
   (g) 5′-SEQ ID NO: 29-GOI-linker sequence-IGS-SEQ ID NO: 19-SEQ ID NO: 28-3′;
   (h) 5′-SEQ ID NO: 31-GOI-linker sequence-IGS-SEQ ID NO: 19-SEQ ID NO: 30-3′;
   (i) 5′-UCG-GOI-Linker sequence-IGS-SEQ ID NO: 17-GA-3′;
   (j) 5′-SEQ ID NO: 42-GOI-Linker sequence-IGS-SEQ ID NO: 17-SEQ ID NO: 43-3′;
   (k) 5′-GAG-GOI-Linker sequence-IGS-SEQ ID NO: 17-UC-3′; or
   (l) 5′-SEQ ID NO: 54-GOI-Linker sequence-IGS-SEQ ID NO: 48-SEQ ID NO: 55-3′
   wherein
   the IGS comprises a sequence ‘GNNNNN’ and the target site comprises a sequence ‘nnnnnu’ or
   the IGS comprises a sequence ‘ANNNNN’ and the target site comprises a sequence ‘nnnnnc’,
   wherein ‘NNNNN’ and ‘nnnnn’ are reverse complementary, and the linker sequence is as defined in any one of 1.32-1.34.
1.39. Any forgoing RNA construct, wherein the circular RNA does not contain an exogenous exon sequence.
1.40. Any forgoing RNA, wherein the circular RNA substantially consists of or consists of the GOI.
1.41. Any foregoing RNA construct, wherein the RNA construct does not comprise uridine, but comprises nucleosides selected from pseudouridine (YΨ), 1-methylpseudouridine (1 mΨ), 5-methoxyuridine (5 moU), 2-thiouridine, or 4-thiouridine in place of uridine.
1.42. Any foregoing RNA construct, wherein the circular RNA is formed by connecting the nucleotide at ωN+1 position and the 3′ end nucleotide of the target site in the RNA construct; preferably, ωN is guanine.
1.43. Any foregoing RNA construct, wherein the circular RNA comprises a noncoding sequence having a biological activity, optionally wherein the noncoding sequence is micro RNA or long non-coding (lnc) RNA.
1.44. Any foregoing RNA construct, wherein the circular RNA comprises a protein-coding sequence, optionally wherein the protein-coding sequence is operably linked to an internal ribosome entry site (IRES) sequence.
1.45. Any foregoing RNA construct, wherein the circular RNA comprises an IRES; e.g., wherein the IRES sequence is intact within the nucleotide sequence of interest or is split at either end of the nucleotide sequence of interest but joined after circularization.
1.46. Any foregoing RNA construct, wherein the circular RNA comprises an IRES, wherein the IRES sequence is selected from an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, Human rhinovirus B, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Human enterovirus B, Equine rhinitis virus, Ectropis obliqua picoma-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n-myc, Mouse Gtx, Human p27kip1, Human PDGF2/c-sis, Human p53, Human Pim-1, Mouse Rbm3, Drosophila reaper, Canine Scamper, Drosophila Ubx, Salivirus, Cosavirus, Parechovirus, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2).
1.47. Any foregoing RNA construct, wherein the circular RNA comprises an IRES, wherein the IRES sequence is an IRES sequence of Human rhinovirus B.

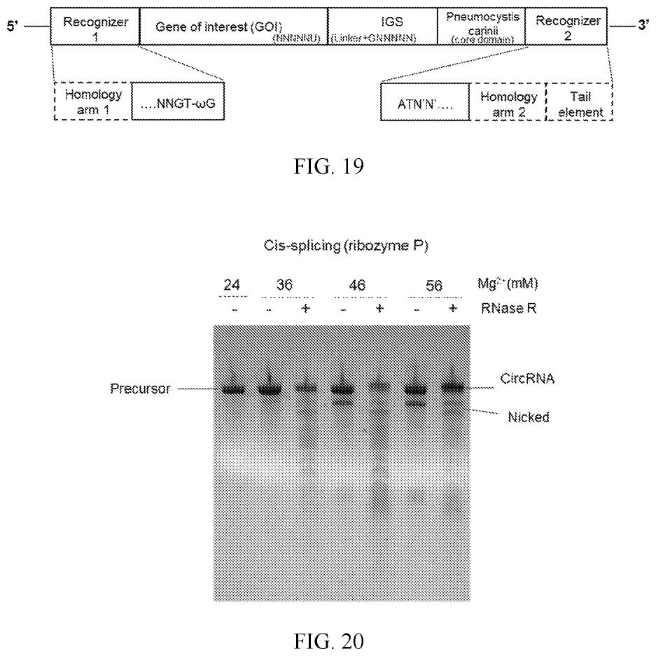

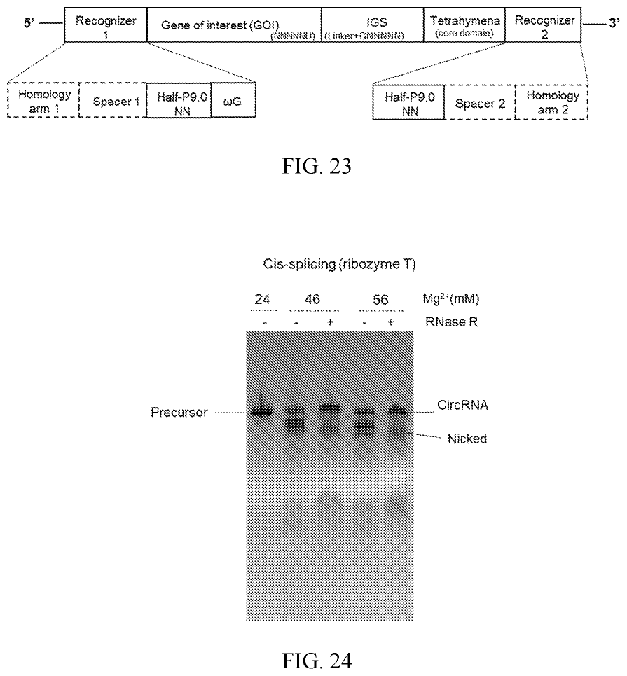

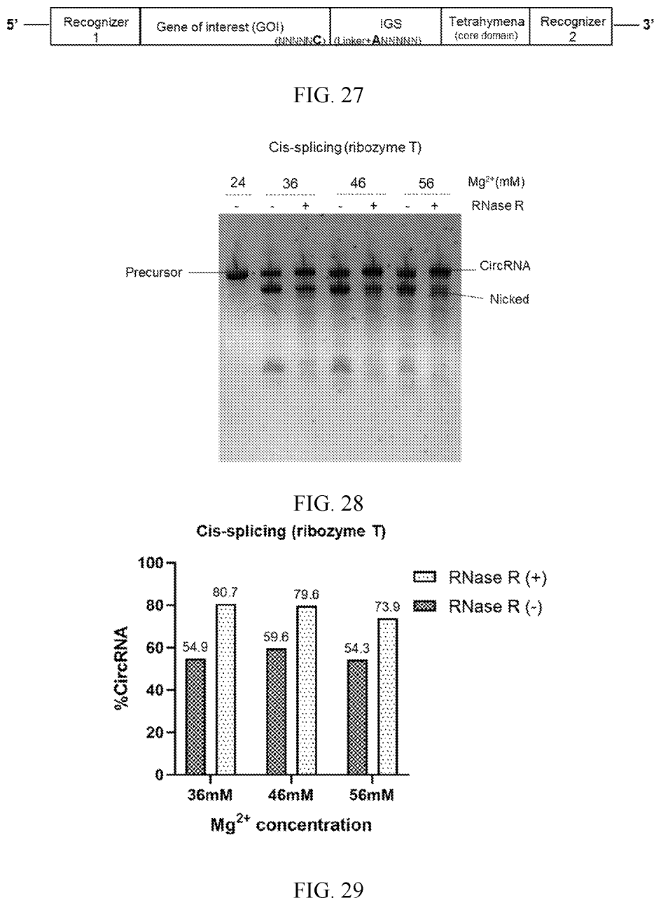

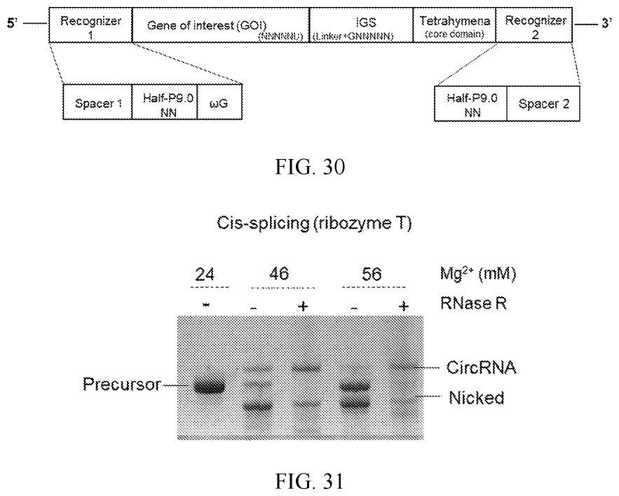

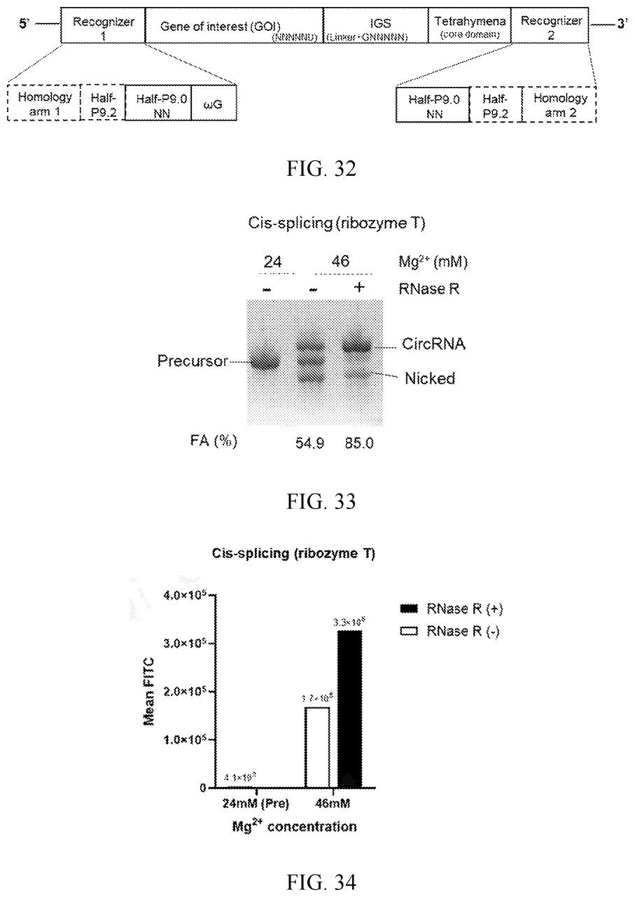

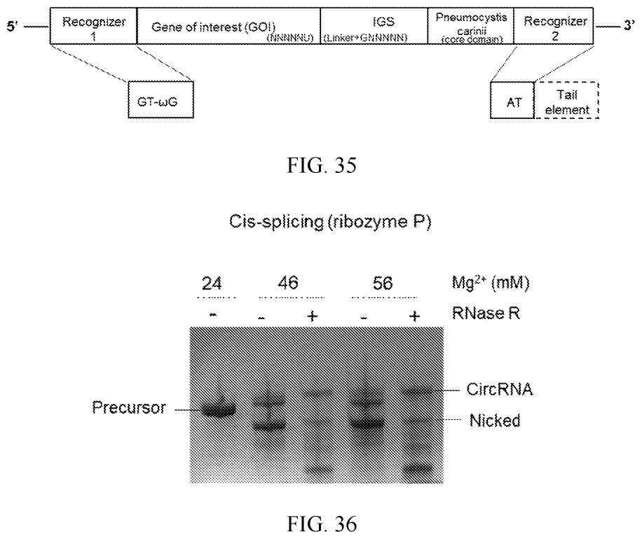

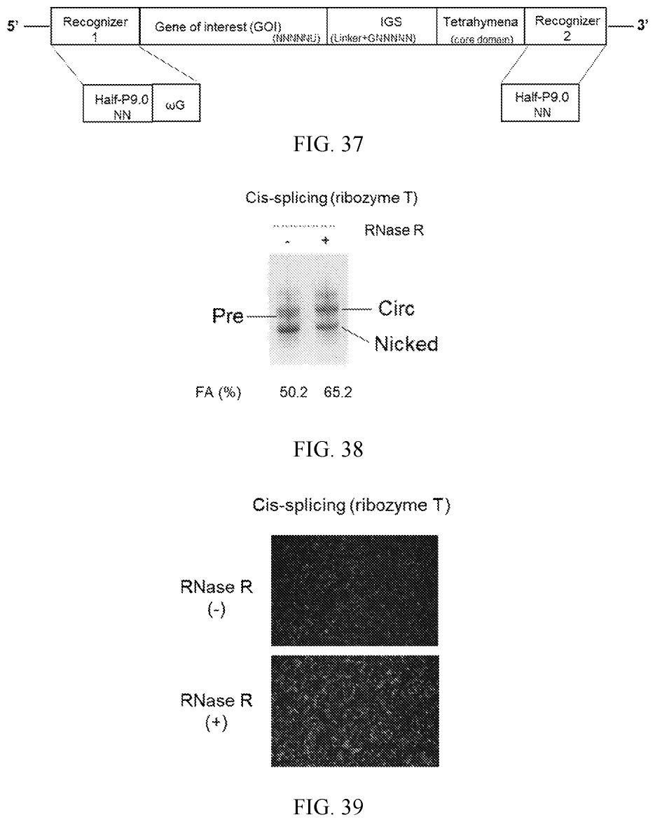

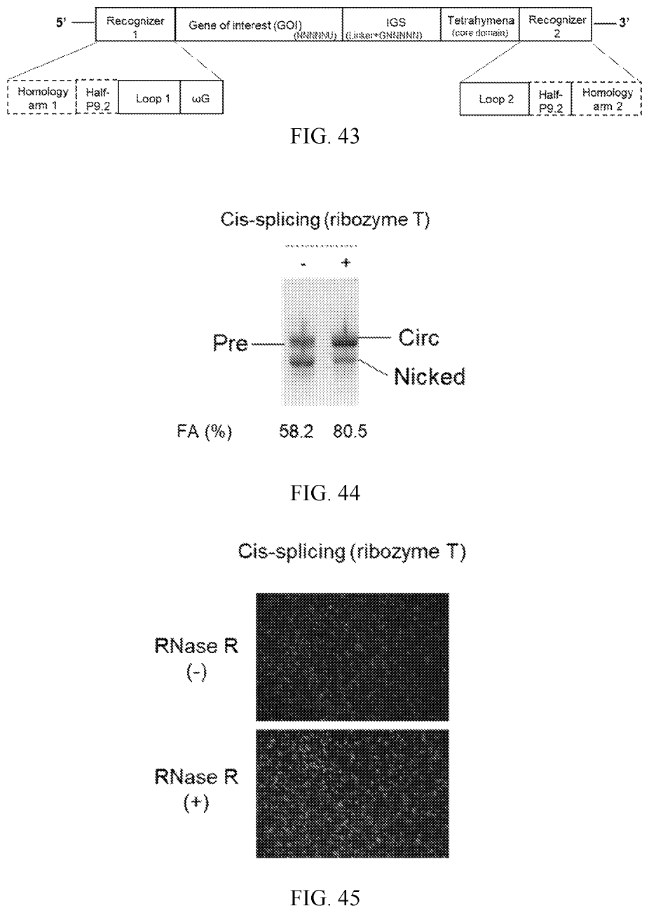

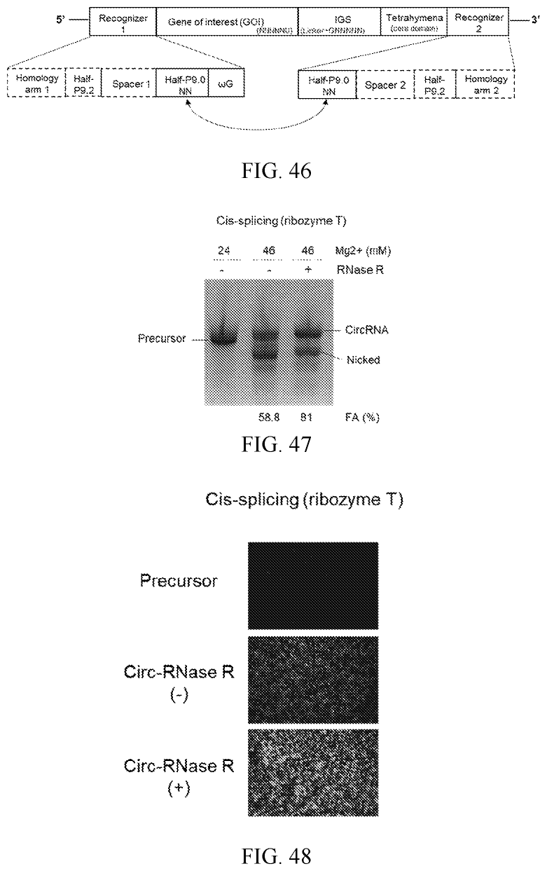

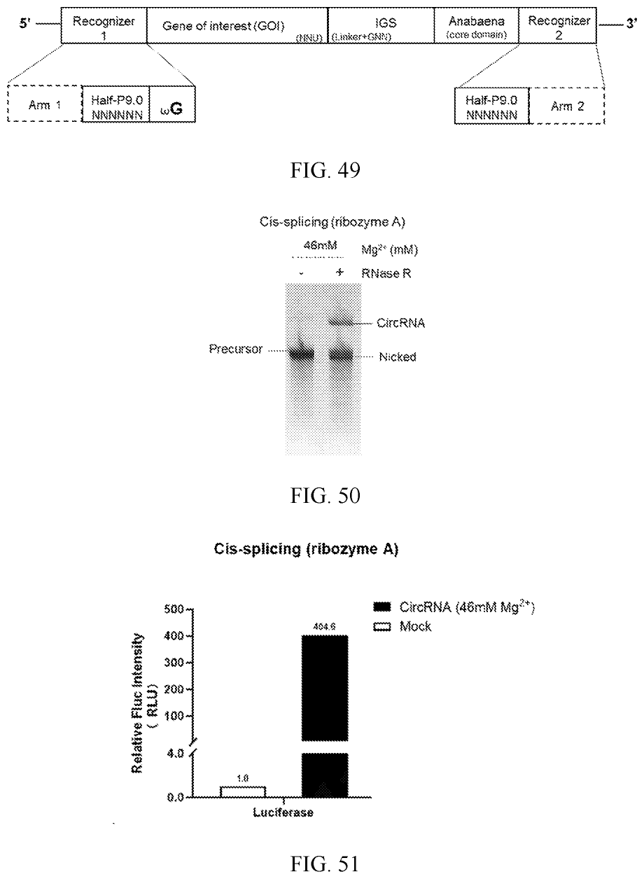

In a particular embodiment, the RNA construct of the present disclosure has a sequence selected from:
(a) 5′-[R1: SEQ ID NO: 21]-[GOI, wherein the target site is located within the ORF and has a nucleotide sequence of ‘nnnnnu’]-[Linker sequence]-[IGS having a nucleotide sequence of ‘GNNNNN’]-[ribozyme core sequence: SEQ ID NO: 17]-[R2: SEQ ID NO: 20]-polyadenylation sequence-3′ [i.e. framework of SEQ ID NO: 1, related to FIG. 6A];
(b) 5′-[R1: SEQ ID NO: 21]-[GOI, wherein the target site is located within the IRES and has a nucleotide sequence of ‘nnnnnu’]-[Linker sequence]-[IGS having a nucleotide sequence of ‘GNNNNN’]-[ribozyme core sequence: SEQ ID NO: 17]-[R2: SEQ ID NO: 20]-polyadenylation sequence-3′ [i.e. framework of SEQ ID NO: 2, related to FIG. 6B];
(c) 5′-[R1: SEQ ID NO: 21]-[GOI, wherein the target site is located within the IRES and has a nucleotide sequence of ‘nnnnnc’]-[Linker sequence]-[IGS having a nucleotide sequence of ‘ANNNNN’]-[ribozyme core sequence: SEQ ID NO: 17]-[R2: SEQ ID NO: 20]-polyadenylation sequence-3′ [i.e. framework of SEQ ID NO: 8, related to FIG. 27];
(d) 5′-[R1: SEQ ID NO: 23]-[GOI, wherein the target site is located within the IRES and has a nucleotide sequence of ‘nnnnnu’]-[Linker sequence]-[IGS having a nucleotide sequence of ‘GNNNNN’]-[ribozyme core sequence: SEQ ID NO: 17]-[R2: SEQ ID NO: 22]-polyadenylation sequence-3′ [i.e. framework of SEQ ID NO: 7, related to FIG. 23];
(e) 5′-[R1: SEQ ID NO: 25]-[GOI, wherein the target site is located within the ORF and has a nucleotide sequence of ‘nnnnnu’]-[Linker sequence]-[IGS having a nucleotide sequence of ‘GNNNNN’]-[ribozyme core sequence: SEQ ID NO: 17]-[R2: SEQ ID NO: 24]-polyadenylation sequence-3′ [i.e. framework of SEQ ID NO: 9, related to FIG. 30];
(f) 5′-[R1: SEQ ID NO: 27]-[GOI, wherein the target site is located within the ORF and has a nucleotide sequence of ‘nnnnnu’]-[Linker sequence]-[IGS having a nucleotide sequence of ‘GNNNNN’]-[ribozyme core sequence: SEQ ID NO: 17]-[R2: SEQ ID NO: 26]-polyadenylation sequence-3′ [i.e. framework of SEQ ID NO: 10, related to FIG. 32];
(g) 5′-[R1: ‘GUG’]-[GOI, wherein the target site is located within the IRES and has a nucleotide sequence of ‘nnnnnu’]-[Linker sequence]-[IGS having a nucleotide sequence of ‘GNNNNN’]-[ribozyme core sequence: SEQ ID NO: 19]-[R2: ‘AU’]-polyadenylation sequence-3′ [i.e. framework of SEQ ID NO: 11, related to FIG. 35];
(h) 5′-[R1: SEQ ID NO: 29]-[GOI, wherein the target site is located within the IRES and has a nucleotide sequence of ‘nnnnnu’]-[Linker sequence]-[IGS having a nucleotide sequence of ‘GNNNNN’]-[ribozyme core sequence: SEQ ID NO: 19]-[R2: SEQ ID NO: 28]-polyadenylation sequence-3′ [i.e. framework of SEQ ID NO: 3, related to FIGS. 2 and 10];
(i) 5′-[R1: SEQ ID NO: 31]-[GOI, wherein the target site is located within the IRES and has a nucleotide sequence of ‘nnnnnu’]-[Linker sequence]-[IGS having a nucleotide sequence of ‘GNNNNN’]-[ribozyme core sequence: SEQ ID NO: 19]-[R2: SEQ ID NO: 30]-polyadenylation sequence-3′ [i.e. framework of SEQ ID NO: 6, related to FIG. 19];
(j) 5′-[R1: ‘UCG’]-[GOI, wherein the target site is located within the ORF and has a nucleotide sequence of ‘nnnnnu’]-[Linker sequence]-[IGS having a nucleotide sequence of ‘GNNNNN’]-[ribozyme core sequence: SEQ ID NO: 17]-[R2: ‘GA’]-polyadenylation sequence-3′ [i.e. framework of SEQ ID NO: 39, related to FIG. 37];
(k) 5′-[R1: SEQ ID NO: 42]-[GOI, wherein the target site is located within the ORF and has a nucleotide sequence of ‘nnnnnu’]-[Linker sequence]-[IGS having a nucleotide sequence of ‘GNNNNN’]-[ribozyme core sequence: SEQ ID NO: 17]-[R2: SEQ ID NO: 43]-polyadenylation sequence-3′ [i.e. framework of SEQ ID NO: 41, related to FIG. 43];
(l) 5′-[R1: ‘GAG’]-[GOI, wherein the target site is located within the ORF and has a nucleotide sequence of ‘nnnnnu’]-[Linker sequence]-[IGS having a nucleotide sequence of ‘GNNNNN’]-[ribozyme core sequence: SEQ ID NO: 17]-[R2: ‘UC’]-polyadenylation sequence-3′ [i.e. framework of SEQ ID NO: 44, related to FIG. 46]; and
(m) 5′-[R1: SEQ ID NO: 54]-[GOI, wherein the target site is located within the 3′ UTR and has a nucleotide sequence of ‘nnu’ ]-[Linker sequence]-[IGS having a nucleotide sequence of ‘GNN’]-[ribozyme core sequence: SEQ ID NO: 48]-[R2: SEQ ID NO: 55]-polyadenylation sequence-3′ [i.e. framework of SEQ ID NO: 45, related to FIG. 49];
   preferably, the linker sequence comprises a sequence that can pair with a sequence in the 5′ region of the GOI to form a P10 duplex mimic.

The RNA construct of the present disclosure may be synthesized in vivo or in vitro by transcription of a template DNA. For example, the DNA template may comprise a promoter upstream of the region that encodes the RNA construct. The promoter may be selected to enable transcription of the RNA construct in prokaryotic or eukaryotic cells. The promoter is recognized by an RNA polymerase, for example a T7 promoter, which is recognized by T7 virus RNA polymerase. In some embodiments, the promoter is a T7 promoter and the RNA polymerase is a T7 virus RNA polymerase; or the promoter is a T6 promoter, and the polymerase is a T6 virus RNA polymerase; or the promoter is an SP6 virus RNA polymerase promoter and the polymerase is SP6 virus RNA polymerase; or the promoter is T3 virus RNA polymerase promoter and the polymerase is T3 virus RNA polymerase; or the promoter is T4 virus RNA polymerase promoter and the polymerase is T4 virus RNA polymerase. In certain embodiments, the RNA polymerase promoter is a T7 virus RNA polymerase promoter and the polymerase is a T7 virus RNA polymerase. Other examples of promoters may include but are not limited to cytomegalovirus (CMV) immediate early promoter, eukaryotic translation elongation factor 1 α (EF-1α) promoter, simian virus 40 (SV40), U6 promoter, H1 promoter, chicken β-actin (CBA) promoter and human phosphoglycerate kinase 1 (hPGK) promoter.

The template DNA may be linear or circular. In some embodiments, the template DNA is prepared by linearizing a DNA plasmid, e.g., by a restriction enzyme. In other embodiments, the template is circular (e.g., a DNA plasmid). The template DNA may comprise an RNA polymerase terminator sequence element downstream of the region that encodes the RNA construct, especially when the template DNA is circular.

The template DNA comprises a sequence encoding the RNA construct, which as described above, is a linear RNA molecule that can self-splice, thereby producing a circular RNA (circRNA). The RNA construct contains the circRNA sequence plus splicing sequences (e.g., ribozyme core sequence and 5′ and 3′ recognizer sequences) necessary to circularize the RNA. These splicing sequences are removed from the RNA construct during the circularization, leaving a circRNA comprising the nucleotide sequence of interest. In some embodiments, the nucleoside moieties in the RNA construct are naturally occurring nucleosides, e.g., adenosine, guanosine, cytidine and uridine. In other embodiments, the nucleoside moieties in the RNA construct comprise nucleosides in addition to or in place of adenosine, guanosine, cytidine and uridine; for example the nucleosides comprise pseudouridine (Ψ), 1-methylpseudouridine (1 mΨ), 2-thiouridine, 4-thiouridine, 5-methoxyuridine (5 moU), 5-methylcytidine, N₆-methyladenosine, inosine or a combination thereof, for example where uridine is replaced with pseudouridine, 1-methylpseudouridine, 2-thiouridine, 4-thiouridine or 5-methoxyuridine (5 moU), and/or cytidine is replaced with 5-methylcytidine, and/or adenosine is replaced with N6-methyladenosine, and/or guanosine is replaced with inosine.

In some embodiments, the DNA template comprises a promoter recognized by an RNA polymerase operably linked to a sequence encoding an RNA construct as described above. As used herein, the phrase “operably linked” means that the elements are positioned on the DNA template such that the RNA construct can be synthesized by in vitro or in vivo transcription of the template DNA. The RNA construct can then form the desired circRNA, e.g., using the methods disclosed herein.

The disclosure thus further provides a DNA construct, e.g., a plasmid, comprising a sequence encoding the RNA construct of the present disclosure, operably linked to a promoter.

The disclosure further provides methods for production of a circRNA by (i) in vitro transcription of a DNA construct, e.g., a plasmid, comprising a sequence encoding the RNA construct of the present disclosure, and (ii) circularization (i.e., self-splicing) of the RNA construct thus transcribed, in a buffered reaction solution comprising magnesium and ingredients required for in vitro transcription, e.g., an RNA polymerase, an RNase inhibitor, ATP, GTP, CTP, UTP, DTT, and a monovalent cation (Na⁺ or K⁺). Optionally, this method is carried out in one step, without a need to purify the RNA construct before allowing the RNA construct to self-splice. In other words, the in vitro transcription and the circularization occur in the same reaction solution at the same reaction conditions (e.g., temperature). Therefore, the reaction solution and reaction conditions must be optimized for the efficiency of both in vitro transcription and circularization.

As is shown in the examples below, the efficiency of the self-splicing and release of the circRNA requires optimal concentrations of magnesium ion. In some embodiments, the reaction solution comprises Mg²⁺ at the concentration greater than 26 mM, e.g., greater than 30 mM or greater than 35 mM. In some embodiments, the concentration of Mg²⁺ in the solution is from 30 mM to 100 mM, e.g., from 30 mM to 90 mM, from 30 mM to 80 mM, from 30 mM to 70 mM, from 30 mM to 60 mM, from 30 mM to 50 mM, from 30 mM to 40 mM, from 35 mM to 100 mM, from 35 mM to 90 mM, from 35 mM to 80 mM, from 35 mM to 70 mM, from 35 mM to 60 mM, from 35 mM to 50 mM, from 35 mM to 40 mM, from 38 to 66 mM, e.g., about 38 mM. In certain embodiments, the concentration of Mg²⁺ in the solution is from 38 mM to 66 mM.

In some embodiments, the reaction solution comprises a pyrophosphatase at the concentration of from 1 U/ml to 5 U/ml, e.g., from 1 U/ml to 4 U/ml, from 1.5 U/ml to 3 U/ml, from 1.5 U/ml to 2.5 U/ml, about 1 U/ml, about 2 U/ml, or about 4 U/ml. As used herein, 1 U (unit) of pyrophosphatase is defined as the amount of enzyme that generates 1 μmol of phosphate per minute from inorganic pyrophosphate under standard reaction conditions (a 10 minute reaction at 25° C. in 20 mM Tris-HCl, pH 8.0, 2 mM MgCl₂ and 2 mM PPi).

The reaction solution further comprises ingredients required for in vitro transcription. In some embodiments, the reaction solution comprises an RNA polymerase, an RNase inhibitor, ATP, GTP, CTP, UTP, DTT, and a monovalent cation (Na⁺ or K⁺). In certain embodiments, the reaction solution comprises about 5 U/μl RNA polymerase, about 1 U/μl RNAse inhibitor, about 10 mM ATP, about 10 mM GTP, about 10 mM CTP, about 10 mM UTP, about 10 mM DTT, and 5 mM monovalent cation (Na⁺ or K⁺). The reaction solution may comprise a buffer. The pH of the reaction solution may be from 6 to 8, e.g., from 7 to 8, or about 7.5.

The RNA construct may be unmodified, partially modified or completely modified. In some embodiments, the RNA construct is unmodified, i.e., contains only naturally occurring nucleotides. In other embodiments, the RNA construct is partially modified or completely modified. A part or all of at least one ribonucleoside triphosphate in the reaction solution may be replaced with a modified nucleoside triphosphate in order to synthesize partially modified or completely modified RNA construct. Examples of modified nucleoside triphosphate include, but are not limited to, pseudouridine-5′-triphosphate, 1-methylpseudouridine-5′-triphosphate, 2-thiouridine-5′-triphosphate, 4-thiouridine-5′-triphosphate and 5-methylcytidine-5′-triphosphate.

RNA polymerase used for in vitro transcription may be chosen based on the RNA polymerase promoter in the DNA template. For example, if the RNA polymerase promoter in the DNA template is a T7 virus RNA polymerase promoter, the reaction solution may comprise a T7 RNA polymerase. In some embodiments, the reaction solution comprises an RNA polymerase selected from T7 virus RNA polymerase, T6 virus RNA polymerase, SP6 virus RNA polymerase, T3 virus RNA polymerase, or T4 virus RNA polymerase. In certain embodiments, the RNA polymerase promoter in the DNA template is a T7 virus RNA polymerase and the reaction solution comprises a T7 virus RNA polymerase.

In some embodiments, the in vitro transcription of the template DNA and the circularization (i.e., self-splicing) of the RNA construct are carried out at a temperature of from 37° C. to 55° C., e.g., from 39° C. to 55° C., from 41° C. to 55° C., from 43° C. to 55° C., from 37° C. to 50° C., from 39° C. to 50° C., from 41° C. to 50° C., from 43° C. to 50° C., from 37° C. to 47° C., from 39° C. to 47° C., from 41° C. to 47° C., from 43° C. to 47° C., from 47° C. to 55° C., from 50° C. to 55° C., from 39° C. to 43° C., about 37° C., about 39° C., about 41° C., about 43° C., about 47° C., about 53° C., or about 55° C. It has been found that the production of a major by-product, dsDNA, is reduced with increasing temperature. dsRNA can be recognized by cytosolic sensors such as RIG-I and MDA5 and then activate the innate immune system (Wu et al., 2020, “Synthesis of low immunogenicity RNA with high-temperature in vitro transcription, RNA 26, 345-360; Olejniczak, 2010, “Sequence-non-specific effects of RNA interference triggers and microRNA regulators, Nucleic Acids Res 38, 1-16). Since ds RNA production should be reduced as much as possible, a temperature higher than 37° C. is preferred. In some embodiments, the in vitro transcription of the template DNA and the circularization (i.e., self-splicing) of the RNA construct are carried out at a temperature higher than 37° C., e.g., from 39° C. to 55° C., from 41° C. to 55° C., from 43° C. to 55° C., from 39° C. to 50° C., from 41° C. to 50° C., from 43° C. to 50° C., from 39° C. to 47° C., from 41° C. to 47° C., from 43° C. to 47° C., from 47° C. to 55° C., from 50° C. to 55° C., from 39° C. to 43° C., about 39° C., about 41° C., about 43° C., about 47° C., about 53° C., or about 55° C.

A genetically modified RNA polymerase exhibiting increased thermo stability (e.g., T7 Toyobo) may be preferred if the in vitro transcription of the template DNA and the circularization (i.e., self-splicing) of the RNA construct are carried out at a high temperature. In some embodiments, the in vitro transcription of the template DNA and the circularization (i.e., self-splicing) of the RNA construct are carried out at a temperature of from 47° C. to 55° C., e.g., from 50° C. to 55° C., about 47° C., about 53° C., or about 55° C. and the RNA polymerase is a thermostable polymerase (e.g., T7 Toyobo).

The in vitro transcription of the template DNA and the circularization (i.e., self-splicing) of the RNA construct may be carried out for at least 1 hour, e.g., at least 1.5 hours, at least 2.5 hours, at least 3 hours, from 1 hour to 3 hours, from 1.5 hours to 3 hours, from 2 hours to 3 hours, or from 2.5 hours to 3 hours. The reaction time no less than 1.5 hours is preferred to guarantee the sufficient circularization. On the other hand, the prolongation of the reaction time has the potential to increase by-products. Therefore, the optimal reaction duration of the one-step process may be 2.5-3 hours. In a preferred embodiment, the in vitro transcription of the template DNA and the circularization (i.e., self-splicing) of the RNA construct are carried out for 2.5-3 hours.

In some embodiments, the method further comprises a step of removing the DNA template after the self-splicing of the RNA construct. The DNA template may be removed by adding a DNase I, e.g., for 30 min at 37° C.

In some embodiments, the method further comprises a step of purifying the circular RNA after the self-splicing of the RNA construct or after the step of removing the DNA template, if the method comprises a step of removing the DNA template. In some embodiments, the purification step is selected from a precipitation step, a tangential flow filtration step and a chromatographic step, and a combination thereof. The precipitation step may be an alcoholic precipitation step or LiCl precipitation. The tangential flow filtration step may be a diafiltration step using tangential flow filtration and/or a concentration step using tangential flow filtration. The chromatographic step may be selected from HPLC, anion exchange chromatography, affinity chromatography, hydroxyapatite chromatography, magnetic bead chromatography and core bead chromatography. In some embodiments, the purification step comprises a precipitation step, e.g., LiCl precipitation. In other embodiments, the purification step comprises a chromatography, e.g., magnetic bead chromatography.

The disclosure thus provides, in an aspect, a method of preparing a circular RNA (Method 1), comprising (i) providing a template DNA, wherein the template DNA comprises a sequence encoding the RNA construct of the present disclosure, operably linked to a promoter, in a reaction solution, thereby allowing synthesis of the RNA construct by in vitro transcription of the template DNA and allowing the RNA construct to self-splice, to produce a circular RNA, and (ii) recovering the circular RNA thus produced.

For example, the invention includes:
1.1. Method 1, wherein the in vitro transcription of the template DNA and the self-splicing (i.e., circularization) of the RNA construct are carried out in the same reaction solution under the same reaction conditions (e.g., the same reaction temperature).
1.2. Any foregoing Method wherein the method does not comprise a step of purifying the RNA construct before allowing the RNA construct to self-splice.
1.3. Any foregoing Method wherein the template DNA is circular, optionally wherein the circular template DNA is a DNA plasmid.
1.4. Method 1, 1.1 or 1.2, wherein the template DNA is linear, optionally wherein the linear template DNA is prepared by linearizing a DNA plasmid, e.g., by a restriction enzyme.
1.5. Any of preceding methods, wherein the promoter is a T7 virus RNA polymerase promoter, T6 virus RNA polymerase promoter, SP6 virus RNA polymerase promoter, T3 virus RNA polymerase promoter, or T4 virus RNA polymerase promoter.
1.6. Any of preceding methods, wherein the promoter is a T7 virus RNA polymerase promoter.
1.7. Any of preceding methods, wherein the reaction solution comprises Mg²⁺ at the concentration greater than 26 mM, e.g., greater than 30 mM or greater than 35 mM.
1.8. Any of preceding methods, wherein the concentration of Mg²⁺ in the solution is from 30 mM to 100 mM, e.g., from 30 mM to 90 mM, from 30 mM to 80 mM, from 30 mM to 70 mM, from 30 mM to 60 mM, from 30 mM to 50 mM, from 30 mM to 40 mM, from 35 mM to 100 mM, from 35 mM to 90 mM, from 35 mM to 80 mM, from 35 mM to 70 mM, from 35 mM to 60 mM, from 35 mM to 50 mM, from 35 mM to 40 mM, from 38 to 66 mM, e.g., about 38 mM, optionally wherein the concentration of Mg²⁺ in the solution is from 38 mM to 66 mM.
1.9. Any of preceding methods, wherein the reaction solution comprises a pyrophosphatase at the concentration of from 1 U/ml to 5 U/ml, e.g., from 1 U/ml to 4 U/ml, from 1.5 U/ml to 3 U/ml, from 1.5 U/ml to 2.5 U/ml, about 1 U/ml, about 2 U/ml, or about 4 U/ml.
1.10. Any of preceding methods, wherein the reaction solution comprises an RNA polymerase, an RNase inhibitor, ATP, GTP, CTP, UTP, DTT, and a monovalent cation (Na⁺ or K⁺).
1.11. Any of preceding methods, wherein the reaction solution comprises 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
1.12. Any of preceding methods, wherein the reaction solution comprises 38-66 mM Mg²⁺, optionally 1-4 U/ml pyrophosphatase, an RNA polymerase, an RNase inhibitor, ATP, GTP, CTP, UTP, DTT, and a monovalent cation (Na⁺ or K⁺).
1.13. Any of preceding methods, wherein the reaction solution comprises 38 mM Mg²⁺, 2 U/ml pyrophosphatase, an RNA polymerase, an RNase inhibitor, ATP, GTP, CTP, UTP, DTT, and a monovalent cation (Na⁺ or K⁺).
1.14. Any of preceding methods, wherein the reaction solution comprises 38-66 mM Mg²⁺, optionally 1-4 U/ml pyrophosphatase, 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
1.15. Any of preceding methods, wherein the reaction solution comprises 38 mM Mg²⁺, 2 U/ml pyrophosphatase, 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
1.16. Any of preceding methods, wherein the reaction solution comprises a buffer.
1.17. Any of preceding methods, wherein the pH of the reaction solution is from 6 to 8, e.g., from 7 to 8, or about 7.5.
1.18. Any of preceding methods, wherein the reaction solution comprises a RNA polymerase selected from T7 virus RNA polymerase, T6 virus RNA polymerase, SP6 virus RNA polymerase, T3 virus RNA polymerase, or T4 virus RNA polymerase.
1.19. Any of preceding methods, wherein the RNA polymerase promoter in the DNA template is a T7 virus RNA polymerase promoter and the reaction solution comprises a T7 virus RNA polymerase.
1.20. Any of preceding methods, wherein the in vitro transcription of the template DNA and the circularization (i.e., self-splicing) of the RNA construct are carried out at a temperature of from 37° C. to 55° C., e.g., from 39° C. to 55° C., from 41° C. to 55° C., from 43° C. to 55° C., from 37° C. to 50° C., from 39° C. to 50° C., from 41° C. to 50° C., from 43° C. to 50° C., from 37° C. to 47° C., from 39° C. to 47° C., from 41° C. to 47° C., from 43° C. to 47° C., from 47° C. to 55° C., from 50° C. to 55° C., from 39° C. to 43° C., about 37° C., about 39° C., about 41° C., about 43° C., about 47° C., about 53° C., or about 55° C., optionally wherein the in vitro transcription of the template DNA and the circularization (i.e., self-splicing) of the RNA construct are carried out at a temperature higher than 37° C., e.g., from 39° C. to 55° C., from 41° C. to 55° C., from 43° C. to 55° C., from 39° C. to 50° C., from 41° C. to 50° C., from 43° C. to 50° C., from 39° C. to 47° C., from 41° C. to 47° C., from 43° C. to 47° C., from 47° C. to 55° C., from 50° C. to 55° C., from 39° C. to 43° C., about 39° C., about 41° C., about 43° C., about 47° C., about 53° C., or about 55° C.
1.21. Any of preceding methods, wherein the in vitro transcription of the template DNA and the circularization (i.e., self-splicing) of the RNA construct are carried out at a temperature of from 47° C. to 55° C., e.g., from 50° C. to 55° C., about 47° C., about 53° C., or about 55° C. and the RNA polymerase is a thermostable polymerase (e.g., T7 Toyobo).
1.22. Any of preceding methods, wherein the in vitro transcription of the template DNA and the circularization (i.e., self-splicing) of the RNA construct are carried out for at least 1 hour, e.g., at least 1.5 hours, at least 2.5 hours, at least 3 hours, from 1 hour to 3 hours, from 1.5 hours to 3 hours, from 2 hours to 3 hours, or from 2.5 hours to 3 hours, optionally wherein the in vitro transcription of the template DNA and the circularization (i.e., self-splicing) of the RNA construct are carried out for 2.5-3 hours.
1.23. Any of preceding methods, wherein the method further comprises a step of removing the DNA template after synthesis of the RNA construct, optionally wherein the DNA template is removed by adding a DNase I, e.g., for 30 min at 37° C.
1.24. Any of preceding methods, wherein the method further comprises a step of purifying the circular RNA thus synthesized, e.g., after the step of removing the DNA template if the method comprises a step of removing the DNA template.
1.25. Any of preceding methods, wherein the method further comprises a step of purifying the circular RNA thus synthesized, e.g.
   a) by precipitating the circular RNA, e.g., wherein the precipitation step is an alcoholic precipitation step or LiCl precipitation, optionally wherein the precipitation step is LiCl precipitation; or
   b) by tangential flow filtration step, e.g. a diafiltration step using tangential flow filtration and/or a concentration step using tangential flow filtration; or
   c) by chromatography, e.g. selected from HPLC, anion exchange chromatography, affinity chromatography, hydroxyapatite chromatography, magnetic bead chromatography, and core bead chromatography, optionally wherein the chromatographic step is magnetic bead chromatography.
1.26. Any of preceding methods, wherein the RNA construct is unmodified, i.e., contains only naturally occurring nucleosides, e.g., contains adenosine, guanosine, cytidine and uridine.
1.27. Any of Methods 1-1.25, wherein the RNA construct is partially modified or completely modified, i.e., contains nucleosides other than or in addition to adenosine, guanosine, cytidine and uridine.
1.28. Method 1.27 wherein the RNA construct comprises nucleosides selected from pseudouridine, 1-methylpseudouridine, 2-thiouridine, 4-thiouridine, 5-methylcytidine, N6-methyladenosine, and a combination thereof.
1.29. Method 1.27, wherein a part or all of the ribonucleoside triphosphates in the reaction solution comprise ribonucleoside triphosphates other than or in addition to adenosine triphosphate (ATP), guanosine triphosphate (GTP), cytidine triphosphate (CTP) and uridine triphosphate (UTP).
1.30. Method 1.29 wherein a part or all of the ribonucleoside triphosphates in the reaction solution comprise modified nucleoside triphosphates, e.g., wherein the modified nucleoside triphosphates are selected from pseudouridine-5′-triphosphate, 1-methylpseudouridine-5′-triphosphate, 2-thiouridine-5′-triphosphate, 4-thiouridine-5′-triphosphate, 5-methylcytidine-5′-triphosphate, N₆-methyladenosine-5′-triphosphate and a combination thereof.
1.31. Method 1.30 wherein the nucleosides in the RNA construct do not comprise uridine, but comprise nucleosides selected from pseudouridine, 1-methylpseudouridine, 2-thiouridine, 4-thiouridine, and a combination thereof.
1.32. Method 1.29 wherein the nucleosides in the RNA construct do not comprise cytidine, but comprise 5-methylcytidine.
1.33. Any foregoing Method wherein the circularization efficiency is at least 70%.
1.34. Any foregoing Method wherein the percentage of dsRNA relative to total RNA in the final product is less than 1%, e.g., less than 0.1%.

The disclosure further provides a circular RNA obtained by Method 1, et seq.

The disclosure further provides a pharmaceutical composition comprising a circular RNA obtained by any of Methods 1, et seq., e.g., a lipid nanoparticle comprising a circular RNA obtained by Method 1, et seq.

The disclosure further provides a pharmaceutical composition comprising a vector containing DNA expressing the RNA construct of the present disclosure.

The disclosure provides the following exemplary embodiments.

Embodiment 1. An RNA construct comprising, from 5′ end to 3′ end,
a first pairing sequence;
a guanine (ωG);
a nucleotide sequence of interest (GOI) comprising a target site at its 3′ end;
an internal guide sequence (IGS);
a nucleotide sequence encoding a ribozyme core which has the catalytic activity of a group I intron ribozyme; and
a second pairing sequence;
wherein
the 5′ end nucleotide of the IGS and the 3′ end nucleotide of the target site form a non-Watson-Crick base pair to define a 5′ splice site;
the first and second pairing sequences form a duplex-containing structure upstream of the ωG to define a 3′ splice site; and
the RNA construct is capable of generating a circular RNA comprising the nucleotide sequence of interest through the catalytic activity of the ribozyme core.

Embodiment 2. The RNA construct according to embodiment 1, wherein the ribozyme core comprises a nucleotide sequence encoding the scaffold domain and catalytic domain of a group I intron; preferably, the ribozyme core comprises or consists of the sequence from the IGS end to the sequence before the P9.0 duplex of a group I intron.

Embodiment 3. The RNA construct according to embodiment 1 or 2, wherein the ribozyme core is derived from a group IC1 (e.g., from Tetrahymena sp. (e.g., T. thermophile, T. cosmopolitanis, T. hyperangularis, T. malaccensis or T. pigmentosa) or Pneumocystis sp. (e.g., Pneumocystis carinii), IC2, IC3 (e.g., from Anabaena sp. PCC7120 or Azoarcus sp. BH72) or IA2 (e.g., from Bacteriophage Twort) intron.

Embodiment 4. The RNA construct according to embodiment 1 or 2, wherein the ribozyme core is derived from a Pneumocystis sp. group I intron; for example, a Pneumocystis sp. group I intron comprising a nucleotide sequence selected from SEQ ID NOs: 32-36; preferably, the ribozyme core is derived from a Pneumocystis carinii group I intron comprising the nucleotide sequence of SEQ ID NO: 32, for example, the ribozyme core comprises or consists of the nucleotide sequence of SEQ ID NO: 19 or a nucleotide sequence having at least 95% sequence identity thereto.

Embodiment 5. The RNA construct according to embodiment 1 or 2, wherein the ribozyme core is derived from a Tetrahymena sp. group I intron; for example, a Tetrahymena thermophila group I intron comprising the nucleotide sequence of SEQ ID NO: 12, for example, the ribozyme core comprises or consists of the nucleotide sequence of SEQ ID NO: 17 or a nucleotide sequence having at least 95% sequence identity thereto.

Embodiment 6. The RNA construct according to any one of embodiments 1-5, wherein the duplex-containing structure comprises one or more base pairs.

Embodiment 7. The RNA construct according to any one of embodiments 1-6, wherein the first and second pairing sequences each independently comprises 2-100 nucleotides; for example, the first pairing sequence comprises 2-20, 2-12, 4-10, 6, 7 or 8 nucleotides; and/or the second pairing sequence comprises 2-100, 5-80, 8-60, 10-50, 15, 20, 30, 40, 50, 60, 70, 80, 90 or 100, preferably 5-80 or 8-60 nucleotides.

Embodiment 8. The RNA construct according to any one of embodiments 1-6, wherein the RNA construct further comprises a 5′ homology arm sequence located upstream of the first pairing sequence and a 3′ homology arm sequence located downstream of the second pairing sequence, and the 5′ and 3′ homology arm sequences are at least partially reverse complementary.

Embodiment 9. An RNA construct comprising, from 5′ end to 3′ end,
a nucleotide sequence ‘(Nₓ)ₛ(N_{y})ₜG’, wherein ‘G’ is guanine (ωG);
a nucleotide sequence of interest comprising a target site at its 3′ end;
an internal guide sequence (IGS);
a nucleotide sequence encoding a ribozyme core which has the catalytic activity of a group I intron ribozyme;
a nucleotide sequence ‘(nₓ)_{w}’; and
wherein
the 5′ end nucleotide of the IGS and the 3′ end nucleotide of the target site form a non-Watson-Crick base pair;
‘Nₓ’, ‘nₓ’, and ‘N_{y}’ are each independently any naturally occurring or modified nucleotide;
t is 0 or an integer of 1-20;
s and w are each independently an integer of 1-200;
‘(Nₓ)ₛ’ and ‘(nₓ)_{w}’ form a duplex-containing structure; and
the RNA construct is capable of generating a circular RNA comprising the nucleotide sequence of interest through the catalytic activity of the ribozyme core.

Embodiment 10. The RNA construct according to embodiment 9, wherein the ribozyme core is as defined in any one of embodiments 2-5.

Embodiment 11. The RNA construct according to embodiment 9 or 10, wherein t is 0 or 1; for example, wherein t is 0, ‘(Nₓ)ₛ(N_{y})ₜG’ is ‘N₂N₁G’; or t is 1, ‘(Nₓ)ₛ(N_{y})ₜG’ is ‘N₂N₁N_{y}G’; and ‘(nₓ)_{w}’ is ‘n₁n₂’; wherein ‘N₁’, ‘n₁’, ‘N₂’, ‘n₂’ and ‘N_{y}’ are each independently any naturally occurring or modified nucleotide, ‘N₁’ and ‘n₁’ form a first base pair, and ‘N₂’ and ‘n₂’ form a second base pair.

Embodiment 12. The RNA construct according to embodiment 9 or 10, wherein the RNA construct further comprises a 5′ homology arm sequence located upstream of the ‘(Nₓ)ₛ(N_{y})ₜG’, and a 3′ homology arm sequence located downstream of the ‘(nₓ)_{w}’, wherein the 5′ and 3′ homology arm sequences are at least partially reverse complementary.

Embodiment 13. An RNA construct comprising, from 5′ end to 3′ end,
a first nucleotide sequence comprising a sequence from a nucleotide ‘N_{q}’ to the 3′ end of a group I intron,
a nucleotide sequence of interest (GOI) comprising a target site at its 3′ end,
an internal guide sequence (IGS), and
a second nucleotide sequence comprising a sequence from the IGS end to a nucleotide ‘Nₚ’ of a group I intron;
wherein
the 5′ end nucleotide of the IGS and the 3′ end nucleotide of the target site form a non-Watson-Crick base pair to define a 5′ splice site;
‘Nₚ’ and ‘N_{q}’ are independently selected from any nucleotide from the 3′ end nucleotide of the 5′ half to the 5′ end nucleotide of the 3′ half of P9.0 duplex of the group I intron, and
‘Nₚ’ is located upstream of ‘N_{q}’ in the group I intron.

Embodiment 14. The RNA construct according to Embodiment 13, wherein the group I intron is a group IC1 (e.g., from Tetrahymena sp. (e.g., T. thermophile, T. cosmopolitanis, T. hyperangularis, T. malaccensis or T. pigmentosa) or Pneumocystis carinii), IC2, IC3 (e.g., from Anabaena sp. PCC7120 or Azoarcus sp. BH72) or IA2 (e.g., from Bacteriophage Twort) intron; preferably, the group I intron is a group IC1 intron, for example, a Pneumocystis sp. or Tetrahymena sp. group I intron, more preferably, the group I intron comprises a nucleotide sequence selected from SEQ ID NOs: 32-36 and SEQ ID NO: 12.

Embodiment 15. The RNA construct according to embodiment 13, wherein the group I intron is a Pneumocystis carinii group I intron comprising the nucleotide sequence of SEQ ID NO: 32, ‘Nₚ’ and ‘N_{q}’ are independently selected from any nucleotide from nucleotide 316 to nucleotide 342 of SEQ ID NO: 32; or the group I intron is a Tetrahymena thermophila group I intron comprising the nucleotide sequence of SEQ ID NO: 12, ‘Nₚ’ and ‘N_{q}’ are independently selected from any nucleotide from nucleotide 313 to nucleotide 411 of SEQ ID NO: 12.

Embodiment 16. The RNA construct according to embodiment 13 or 14, wherein ‘Nₚ’ and ‘N_{q}’ are independently selected from any nucleotide from the 3′ end nucleotide of the 5′ half to the 5′ end nucleotide of the 3′ half of a duplex of the group I intron, wherein the duplex is not a P9.0 duplex; for example, the duplex is a P9a/9b, P9.1, P9.1a or P9.2 duplex, preferably a P9.2 duplex.

Embodiment 17. The RNA construct according to embodiment 16, wherein ‘Nₚ’ and ‘N_{q}’ are located within the region connecting the 5′ half and 3′ half of the duplex; or ‘Nₚ’ is the 3′ end nucleotide of the 5′ half of the duplex and ‘N_{q}’ is the 5′ end nucleotide of the 3′ half of the duplex.

Embodiment 18. The RNA construct according to any one of embodiments 13-17, wherein the RNA construct further comprises a 5′ homology arm sequence located upstream of the first nucleotide sequence and a 3′ homology arm sequence located downstream of the second nucleotide sequence; wherein the 5′ and 3′ homology arm sequences are at least partially reverse complementary.

Embodiment 19. The RNA construct according to any one of embodiments 1-18, wherein the non-Waton-Crick base pair formed between the 5′ end nucleotide of the IGS and the 3′ end nucleotide of the target site is
(a) guanine-uracil (G-u), wherein ‘G’ is the 5′ end nucleotide of the IGS and ‘u’ is the 3′ end nucleotide of the target site; or
(b) adenine-cytosine (A-c), wherein ‘A’ is the 5′ end nucleotide of the IGS and ‘c’ is the 3′ end nucleotide of the target site; or
(c) guanine-adenine (G-a), wherein ‘G’ is the 5′ end nucleotide of the IGS and ‘a’ is the 3′ end nucleotide of the target site.

Embodiment 20. The RNA construct according to any one of embodiments 1-17, wherein the IGS and the target site form a P1 duplex mimic.

Embodiment 21. The RNA construct according to any one of embodiments 1-20, wherein
the IGS has the structure of 5′-X(N)ₘ-3′
the target site has the structure of 5′-(n)ₘx-3′
‘X’ and ‘x’ are the nucleotides that form the non-Watson-Crick base pair,
each ‘N’ and ‘n’ is a nucleotide independently selected from A, G, C and U, and
m is an integer of 2-8, preferably 3-6, most preferably 4-5;
preferably, 5′-(N)ₘ-3′ and 5′-(n)ₘ-3′ are reverse complementary.

Embodiment 22. The RNA construct according to any one of embodiments 1-21, wherein the IGS comprises a sequence ‘GNNNNN’ and the target site comprises a sequence ‘nnnnnu’; or the IGS comprises a sequence ‘ANNNNN’ and the target site comprises a sequence ‘nnnnnc’; wherein ‘NNNNN’ and ‘nnnnn’ are reverse complementary.

Embodiment 23. The RNA construct according to any one of embodiments 1-22, wherein the RNA construct further comprises a linker sequence located between the target site and IGS.

Embodiment 24. The RNA construct according to embodiment 23, wherein the linker sequence comprises an unpaired sequence, wherein the target site, the linker sequence and the IGS form a stem-loop structure.

Embodiment 25. The RNA construct according to embodiment 23, wherein the linker sequence comprises, from 5′ end to 3′ end, a third pairing sequence, a loop sequence and a fourth pairing sequence, wherein the third and fourth pairing sequences form a P1 extension mimic; preferably, the P1 extension mimic comprises 1-3 reverse complementary base pairs.

Embodiment 26. The RNA construct according to any one of embodiments 23-25, wherein the linker sequence comprises a fifth pairing sequence which can pair with a sixth pairing sequence at the 5′ region of the nucleotide sequence of interest to form a P10 duplex mimic; preferably, the P10 duplex mimic comprises 3-10 base pairs.

Embodiment 27. The RNA construct according to embodiment 1, having the structure of:
(a) 5′-SEQ ID NO: 21-GOI-linker sequence-IGS-SEQ ID NO: 17-SEQ ID NO: 20-3′;
(b) 5′-SEQ ID NO: 23-GOI-linker sequence-IGS-SEQ ID NO: 17-SEQ ID NO: 22-3′;
(c) 5′-SEQ ID NO: 25-GOI-linker sequence-IGS-SEQ ID NO: 17-SEQ ID NO: 24-3′;
(d) 5′-SEQ ID NO: 27-GOI-linker sequence-IGS-SEQ ID NO: 17-SEQ ID NO: 26-3′;
(e) 5′-GUG-GOI-linker sequence-IGS-SEQ ID NO: 19-AU-3′;
(f) 5′-ACG-GOI-linker sequence-IGS-SEQ ID NO: 19-GU-3′;
(g) 5′-SEQ ID NO: 29-GOI-linker sequence-IGS-SEQ ID NO: 19-SEQ ID NO: 28-3′; or
(h) 5′-SEQ ID NO: 31-GOI-linker sequence-IGS-SEQ ID NO: 19-SEQ ID NO: 30-3′; wherein
the IGS comprises a sequence ‘GNNNNN’ and the target site comprises a sequence ‘nnnnnu’ or
the IGS comprises a sequence ‘ANNNNN’ and the target site comprises a sequence ‘nnnnnc’,
wherein ‘NNNNN’ and ‘nnnnn’ are reverse complementary, and
the linker sequence is as defined in any one of embodiments 24-26.

Embodiment 28. A DNA construct comprising a sequence encoding an RNA construct according to any one of embodiments 1-27.

Embodiment 29. A method of preparing a circular RNA comprising (i) providing a DNA construct according to embodiment 28 in a reaction solution, thereby allowing synthesis of the RNA construct by in vitro transcription of the DNA construct and allowing the RNA construct to self-splice, to produce a circular RNA, and (ii) recovering the circular RNA thus produced.

### EXAMPLES

### Example 1. Circular RNA Preparation Through Cis-Splicing Reaction

Circular RNA is prepared as follows:

### 1.1) Plasmid Construction:

The DNA sequence encoding a circRNA precursor (precursor sequence, SEQ ID NO: 1) based on a Tetrahymena thermophile group I intron comprising the nucleotide sequence of SEQ ID NO: 12 (hereafter referred to as ribozyme T, comprising a ribozyme core sequence of SEQ ID NO: 17) was chemically synthesized and cloned into an expression vector (Genscript) containing a T7 promoter to generate the template plasmid for in vitro transcription (IVT) of the circRNA precursor. The nucleotide sequence to be circularized (SEQ ID NO: 50) comprises a 5′ UTR comprising an IRES sequence from Human rhinovirus B, an open reading frame (ORF) sequence encoding the green fluorescent protein (GFP) and a 3′ UTR. The backsplicing site is designed inside the ORF (corresponding to FIG. 6A). That is, a sequence of ‘CTATAT’ (‘nnnnnu’) in the ORF is selected as the target site sequence. The nucleotide sequence of interest (GOI) is formed by placing the sequence from the 5′ end nucleotide of SEQ ID NO: 50 to the 3′ end nucleotide of the target site at the 3′ end and the remaining sequence of SEQ ID NO: 50 at the 5′ end. A corresponding IGS of ‘GTATAG’ (‘GNNNNN’) is designed and placed between the GOI and the ribozyme core sequence. A 7-nucleotide sequence ‘GGCCATG’ (P10-2) designed to be complementary with the 7-nucleotide sequence ‘CATGGCC’ (P10-1) downstream the target site sequence in SEQ ID NO: 50 is placed upstream of the IGS. A 3-nucleotide sequence ‘CAT’ (P1-ex-1) designed to be complementary with the 3-nucleotide sequence ‘ATG’ (P1-ex-2) at the 3′ end of P10-2 is placed dowstream the target site. A loop sequence of ‘CACATTTTACA’ is designed and inserted between P1-ex-2 and P10-2. R2 (the 3′ recognizer sequence) is designed to comprise the sequence from the 5′ half of P9.0 to the 5′ half of P9.2 of SEQ ID NO: 12 (the sequence connecting the 5′ half of P9.0 and the 5′ half of P9.2 is also referred to as “Spacer 2” for convenience) and a 3′ homology arm sequence (Arm I), and R1 (the 5′ recognizer sequence) is designed to comprise a 5′ homology arm sequence (Arm I) and the sequence from the 3′ half of P9.2 to ωG of SEQ ID NO: 12 (the sequence connecting the 3′ half of P9.2 and the 3′ half of P9.0 is also referred to as “Spacer 1” for convenience).

### 1.2) In Vitro Transcription and Purification for RNA Construct:

The plasmid linearized by BsaI enzymatic digestion is used as a template for the IVT reaction. A single reaction system (20 μL in total) is prepared as follows: 1 U/μL RNase Inhibitor (Novoprotein E125), 6.67 mM ATP, 20 mM GTP, 6.67 mM CTP, 6.67 mM UTP, 1×Transcription buffer (Novoprotein GMP-EB121 containing 6 mM MgCl₂), 10 mM DTT (Sigma 43816), 4 U/mL Pyrophosphatase Inorganic (Novoprotein GMP-M036), 5 mM NaCl (Invitrogen AM9760G), 18 mM MgCl₂ (Invitrogen M1028), 5 U/μL T7 RNA polymerase (Novoprotein GMP-E121), 25 ng/μL linearized plasmid. IVT is carried out at 37° C. for 3 hours and then was treated by DNase I (Novoprotein GMP-E127) for 30 min at 37° C. to remove DNA templates. The RNA construct is purified by precipitation with 7.5 M LiCl or column purification using a Monarch RNA cleanup kit (NEB). A fragment analyzer is applied to evaluate the products.

### 1.3) Generation and Purification of Circular RNA:

The plasmid linearized by BsaI enzymatic digestion was used as a template for the IVT reaction. A single reaction system (20 μL in total) was prepared as follows: 1 U/μL RNase Inhibitor (Novoprotein E125), 10 mM ATP, 10 mM GTP, 10 mM CTP, 10 mM UTP, 1×Transcription buffer (Novoprotein GMP-EB121; containing 6 mM MgCl₂), 10 mM DTT (Sigma 43816), 4 U/mL Inorganic Pyrophosphatases (Novoprotein GMP-M036), 5 mM NaCl (Invitrogen AM9760G), MgCl₂ (Invitrogen M1028) ranging from 30 mM to 50 mM, 5 U/μL T7 RNA polymerase (KactusBio GMP-T7P-EE101-12), 25 ng/μL linearized plasmid. The reaction was carried out at 37° C. for 3 hours; IVT products were treated with DNase I (Novoprotein GMP-E127) for 30 min at 37° C. to remove DNA templates. RNAs were purified by 7.5 M LiCl precipitation or column purification using a Monarch RNA cleanup kit (NEB).

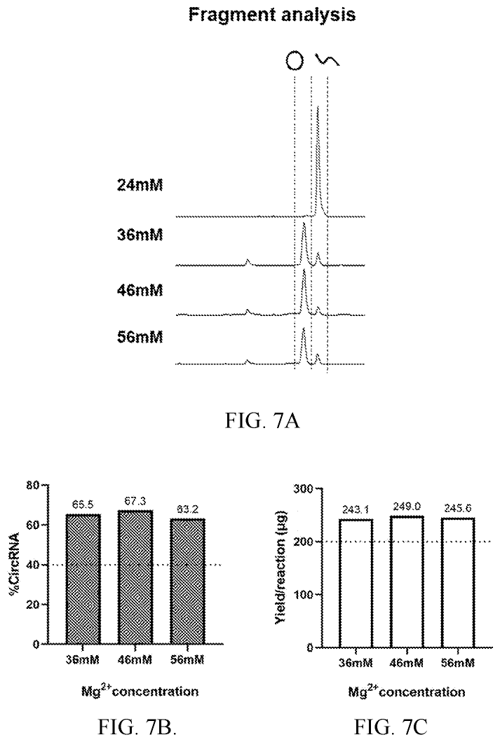

A fragment analyzer (FA) was applied to evaluate the products. Specifically, in the RNA mode, purified circular RNAs were further analyzed with capillary electrophoresis with Agilent 5200 or 5300 Bioanalyzer. Samples were diluted to an appropriate concentration and analyzed according to the manufacturer's instructions (Agilent DNF-471 RNA Kit, 15 nt). Agilent ProSize Data Analysis Software was utilized to analyze the results. The Smear analysis module was applied to identify the peak range corresponding to the circular RNA component. As FA cannot distinguish between circRNA and nicked RNA, both components were exhibited in a single peak before the precursor peak, as shown in FIG. 7A. The percentage of selected peak area in the total area of all detected peaks can be considered the efficiency of precursor splicing. Alternatively, Capillary Quantitative Analysis (PA800 Plus, SCIEX) may be also used for circRNA detection: CircRNA samples dissolved in nuclease-free water were diluted to 10 ng/μL using Sample Loading Solution (SLS) (SCIEX, 608082). For complex components, further denaturation was carried out (70° C., 3 min; ice bath, 2 min). 100 μL of the treated sample was used for analysis. Sample detection was performed on the PA 800 Plus system (SCIEX, A66528) using the RNA 9000 Purity & Integrity Kit (SCIEX, C48231). After sample loading, the components were separated by capillary electrophoresis and detected by the LIF detector (conditions: 50 psi, 30 kV, 25° C., 40 min), resulting in peak signals at different times. The RNA 9000 Ladder (SCIEX, AM7150) was used as a reference for the size of sample bands, and the area percent (%) and Quality (bp) of each component were obtained through integrated quantification.

### 1.4) RNase R Digestion to Remove Linear RNA:

Some samples from the 1.2 and 1.3 reactions were treated with RNase R to verify the generation of circular RNA. A single reaction system (50 μL in total) was prepared as follows: adding 5 μL 10×RNase R reaction buffer (10×: 0.2 M Tris-HCl pH 8.0, 1 M KCl, 1 mM MgCl₂) and RNase R 30 unit to IVT RNAs with a total amount of 10 μg to 50 μg (adjust the volume to 50 μL with water). After incubation at 37° C. for 20 minutes, the products were purified using the Monarch RNA cleanup kit (NEB). In all cases, 150 ng RNA per sample was diluted 1:1 in volume with 2×GLB II (gel loading buffer II, Thermofisher) to a final volume of 20 μl/well, heated to 75° C. for at least 2 min, and cooled on ice for at least 3 min. RNA was then separated on a precast 2% E-Gel EX Agarose Gel (Invitrogen) on an E-Gel Power Snap Electrophoresis System (Invitrogen) using the E-Gel EX 1%-2% program; ssRNA ladder (NEB) was used as a standard. Bands were visualized using blue light transillumination.

### 1.5) Ribozyme T can Effectively Mediate Circularization of the Precursor Through Cis-Splicing:

It is shown that the RNA precursor construct could be directly self-spliced and circularized in the IVT system by adjusting the final concentration of Mg²⁺ greater than 26 mM, such as increasing to a certain range, including but not limited to 36 mM to 56 mM (FIG. 7A). In this case, self-splicing of the precursor resulted in a 1521-nt circular RNA formed by connecting the 3′ end nucleotide of the target site (i.e., the 3′ end nucleotide ‘U’ of the GOI) and the nucleotide immediately downstream of the ωG (i.e., the 5′ end nucleotide ‘C’ of the GOI). RNA sequencing across the putative splice junction of the RNA products after RNase R treatment also confirmed the correct ligation between the 5′ and 3′ ends of the GOI (data not shown). Specifically, to perform circRNA analysis via sequencing, gel-purified RNA was subjected to reverse transcription using a PrimeScript RT Reagent Kit with random primers (TAKARA, RR037B), followed by PCR amplification with primers capable of amplifying transcripts across the splice junction. The resulting PCR products were then subjected to Sanger sequencing in order to validate the backsplice junction of the circular RNA. FA results show the peaks of different products, and the proportion of the circularized product did not further increase with higher Mg²⁺ and was primarily maintained at about 60% (FIG. 7B). In addition, there is no significant change for IVT yield within the concentration range of Mg²⁺ (36 mM-56 mM) (FIG. 7C).

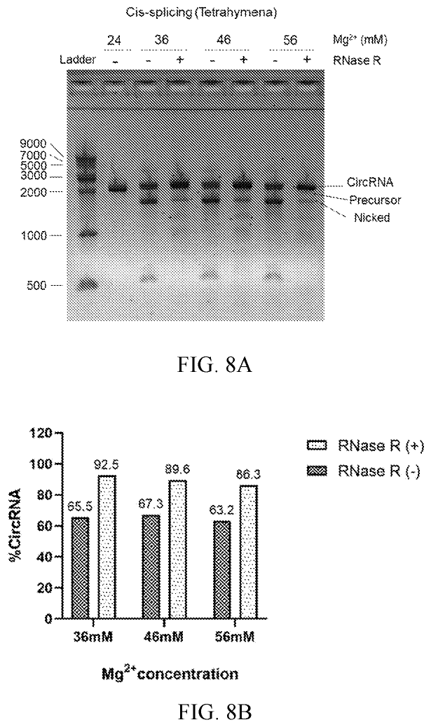

Previous studies reported that the preparation process of circular RNAs is accompanied by the generation of nicked RNAs, which cannot be separated from circular RNAs with equivalent molecular size by traditional electrophoretic methods such as capillary electrophoresis or agarose gel but can be separated and detected by the E-Gel EX system. E-Gel shows a band of nicked RNA under the corresponding band of the precursor (FIG. 8A). Consistent with the results previously reported in the E-Gel system, the migration rate of circular RNA is slower than that of the precursor, while the migration rate of nicked RNA is faster than that of the precursor (FIG. 8A). Furthermore, the digestion of RNase R confirmed the circularization of RNA construct (FIGS. 8A and 8B).

### 1.6) Circular RNA Generated by Cis-Splicing can Express Proteins in Cells:

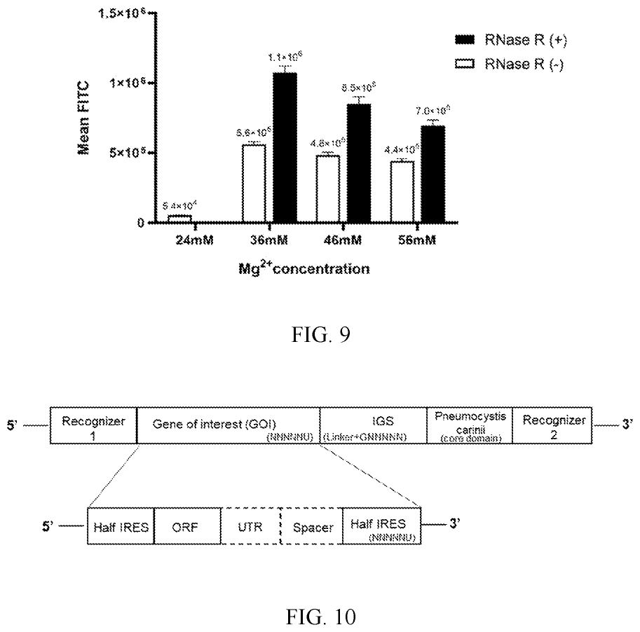

To examine the cellular expression of the circularized RNA with a complete ORF region, the circularization products, including the RNase R-treated samples, were transfected into HEK293 cells with the precursor as a control. Specifically, 50000 cells were seeded per well of a 96-well plate, 100 ng RNA sample was transfected into cells per well using transfection reagent (TransIT, Mirus), and reporter gene expression was detected by flow cytometry 48 h later. The results show that the circularization products and RNase R digested products could effectively express GFP but not for the RNA construct (FIG. 9). Moreover, since most of the linear components had been removed by RNase R digestion, resulting in more circular RNAs transfected into the cells, a final higher expression level than that of untreated samples was observed for the RNase R digested samples (FIG. 9).

### Example 2. Ribozyme T can Mediate the Circularization where the Backsplicing Site is Located in Other Regions, Such as IRES Element

A circRNA precursor (precursor sequence, SEQ ID NO: 2) based on ribozyme T was generated and purified through the same processes described in Examples 1.1 and 1.2. The backsplicing site was designed inside the IRES (FIG. 6B) of the nucleotide sequence to be circularized (SEQ ID NO: 50). That is, a sequence of ‘GCCTTT’ (‘nnnnnu’) in the IRES was selected as the target site sequence. Accordingly, a sequence of ‘GAAGGC’ (‘GNNNNN’) was designed as the IGS. Sequences for the formation of a P10 duplex mimic and a P1 extension mimic were introduced using similar strategy described in Example 1.1. The preparation of circular RNA was carried out following the procedures described in Example 1.3. The circularization products were digested by RNase R as described in Example 1.4.

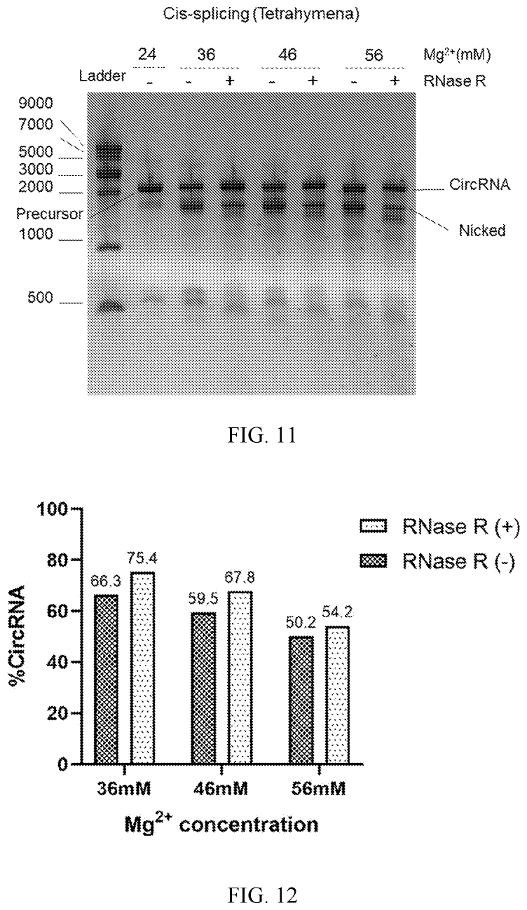

The results show that adjusting magnesium ion concentration, including but not limited to 36 mM to 56 mM, can prompt the precursor in the IVT system to directly undergo a self-splicing reaction (FIG. 11). E-Gel exhibits the corresponding bands for the products of the splicing reaction, where digestion by RNase R could enrich for circular RNAs generated by splicing (FIG. 11). FA analysis shows that the fractions of circular RNAs generated under the indicated Mg²⁺ concentration are all above 50% (FIG. 12). The products were subjected to RNase R digestion, in which the purity of circRNAs was further improved (FIG. 12).

### Example 3. Circularization of RNA can be Mediated by a Different Group I Intron Through Cis-Splicing

A circRNA precursor (precursor sequence, SEQ ID NO: 3) based on a Pneumocystis carinii group I intron (hereinafter referred to as ribozyme P, comprising a ribozyme core sequence of SEQ ID NO: 19) was generated and purified through the same processes described in Examples 1.1 and 1.2 (FIGS. 2 and 10). The backsplicing site was designed inside the IRES (FIG. 10) of the nucleotide sequence to be circularized (SEQ ID NO: 50). The preparation of circular RNA was carried out following the procedures described in Example 1.3. The circularization products were digested by RNase R as described in Example 1.4. In SEQ ID NO: 3, R2 comprises a dinucleotide sequence ‘GU’ (corresponding to the 5′ half of P9.0) and a 3′ homology arm sequence (Arm III), and R1 comprises a 5′ homology arm sequence (Arm III) and a dinucleotide sequence ‘AC’ (corresponding to the 3′ half of P9.0) and ωG.

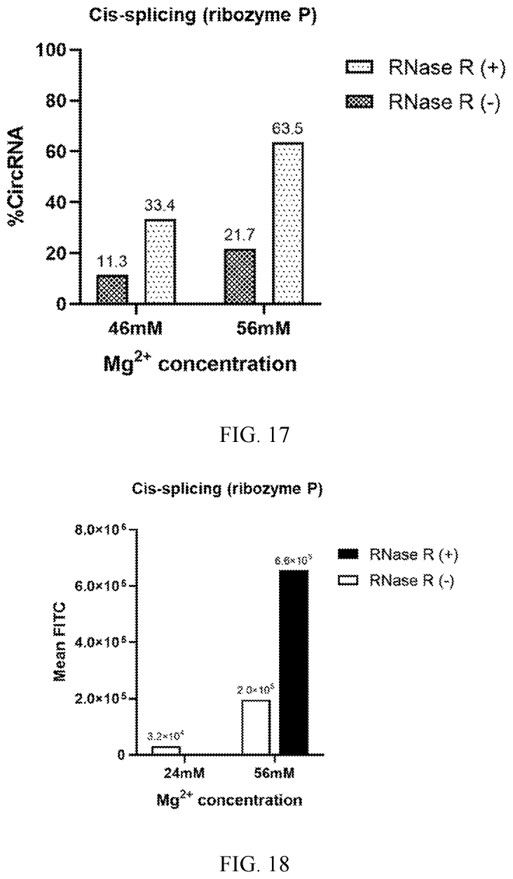

E-gel shows that ribozyme P can catalyze the self-cleavage of the precursor in the IVT reaction (tested Mg²⁺ concentration was 56 mM), and the splicing products were subjected to RNase R digestion to confirm the generation of circular RNA (FIG. 16). FA analysis shows that the increase of Mg²⁺ to 56 mM can promote ribozyme P mediated circularization (FIG. 17). The expression of reaction products in cells was detected according to the method described in Example 1.6. The results show that a cis-splicing system designed based on a different group I intron (here ribozyme P) can also successfully mediate RNA circularization, and the expression was enhanced in cells with improved circRNA purity after digestion by RNase R (FIG. 18).

### Example 4. Splitting the 5′ Splice Site Sequence can Also Mediate Precursor Splicing into circRNA but with Lower Efficiency than when Splitting the 3′ Splice Site Sequence

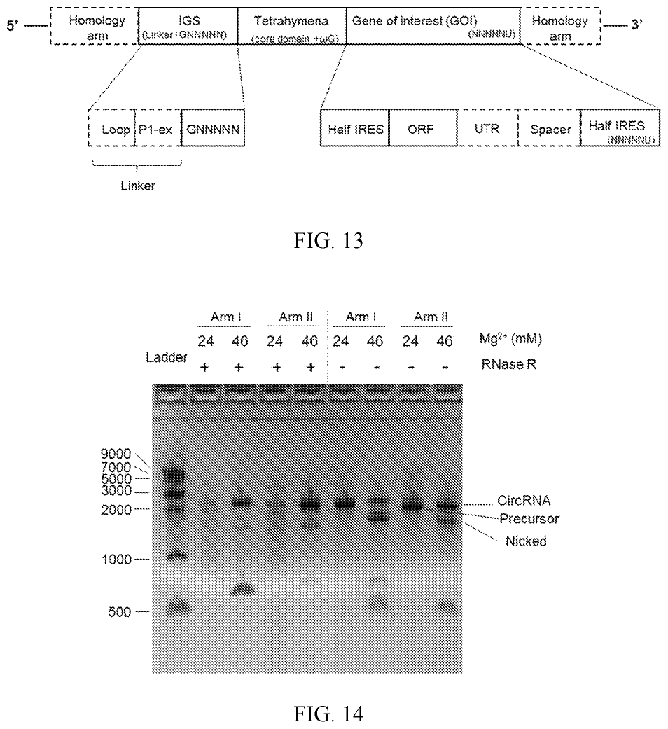

Different from the structures mentioned above (the sequence determining the 3′ splice site was split to the 5′ and 3′ regions of the precursor), in this Example, the sequence determining the 5′ splice site was split to the 5′ and 3′ regions of the precursor (FIG. 13). Accordingly, the ribozyme used in this Example comprises the sequence for P9.0 and P9.2 duplexes at its 3′ end (SEQ ID NO: 18, which comprises the sequence from the IGS end to the ωG of ribozyme T). In order to improve the cis-splicing efficiency, two sets of homology arm sequences of different length (Arm I, longer 5′ and 3′ homology arm sequences; and Arm II, shorter 5′ and 3′ homology arm sequences) were incorporated at both ends of the precursor, which can form homologous arms with partially complementary regions, to generate the circRNA precursors of SEQ ID NOs: 4 and 5 (FIG. 13). The circRNA precursors were generated and purified through the processes described in Examples 1.1 and 1.2. The backsplicing site was designed inside the IRES (FIG. 13) of the nucleotide sequence to be circularized (SEQ ID NO: 50). The preparation of circular RNA was carried out following the procedures described in Example 1.3. The circularization products were digested by RNase R as described in Example 1.4.

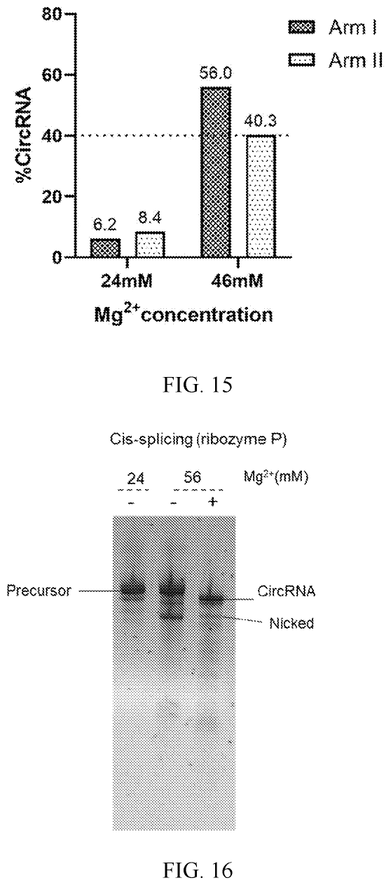

The results show that circularization of the precursor is triggered when adjusting the Mg²⁺ concentration of IVT to include but not limited to a concentration of 46 mM. The digestion of RNase R could remove most of the linear components (like the precursor) and enrich the circular RNA, as shown in E-Gel (FIG. 14). Additionally, FA analysis shows a higher circularization efficiency when using the longer homology arm-Arm I (FIG. 15).

However, compared to the design of splitting the 3′ splice site sequence (FIG. 8B), the design of spliting the 5′ splice site sequence had a lower efficiency (not higher than 60%) (FIG. 15) as well as more by-products shown in E-Gel (FIGS. 8A and 14), despite optimizing the homology arm elements.

### Example 5. P9.0 Mimic Duplexes Containing Wobble Base Pairs are Also Compatible with Ribozyme P-Mediated Circularization

P9.0 duplex is essential for the recognition of the 3′ splice site. In addition to Watson-Crick base pairing in P9.0 as tested in Example 3 (precursor sequence, SEQ ID NO: 3), this Example tested a P9.0 containing a wobble base pair, G-U (FIG. 19). The circRNA precursor (precursor sequence, SEQ ID NO: 6) based on ribozyme P was generated and purified through the processes described in Examples 1.1 and 1.2. The backsplicing site was designed inside the IRES (FIG. 10) of the nucleotide sequence to be circularized (SEQ ID NO: 50). The preparation of circular RNA was carried out following the procedures described in Example 1.3. The circularization products were digested by RNase R as described in Example 1.4. In SEQ ID NO: 6, R2 comprises a dinucleotide sequence ‘AU’ (corresponding to the 5′ half of P9.0) and a 3′ homology arm sequence (Arm III), and R1 comprises a 5′ homology arm sequence (Arm III) and a dinucleotide sequence ‘GU’ (corresponding to the 3′ half of P9.0) and ωG.

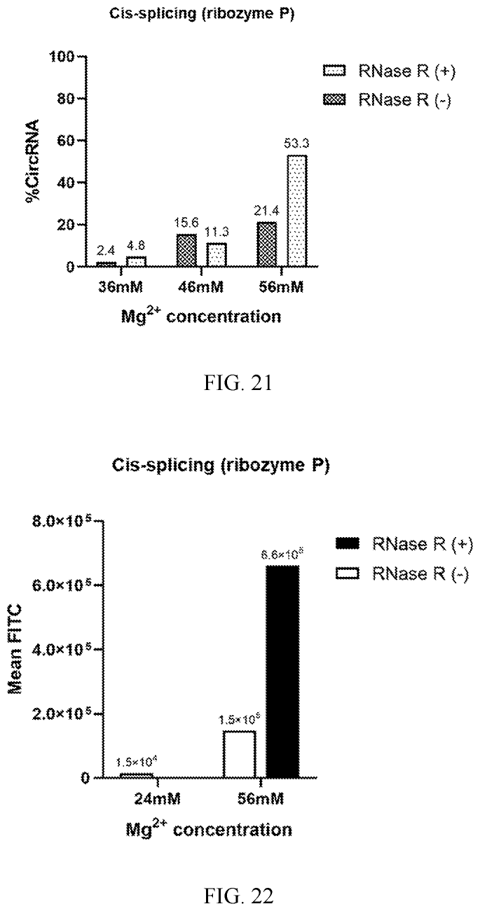

E-Gel shows that a P9.0 containing wobble base pairs could also be compatible with the self-splicing reaction of ribozyme P (tested Mg²⁺ concentrations were from 36 mM to 56 mM) (FIG. 20). The splicing products were subjected to RNase R digestion to confirm the generation of circular RNA (FIG. 20). FA analysis shows that the increase of Mg²⁺ concentration to 56 mM could promote ribozyme P-mediated circularization (FIG. 21). The expression of reaction products in cells was detected according to the method described in Example 1.6. The results show that ribozyme P with wobble base-paired P9.0 could also successfully mediate RNA circularization, and the expression was enhanced in cells with improved circRNA purity after digestion by RNase R (FIG. 22).

### Example 6. P9.2 May Facilitate Ribozyme T-Mediated Circularization but is not an Essential Element

P9.2 duplex facilitates 3′ site splicing. In this Example, the sequences for the 3′ and 5′ halves of P9.2 were removed to study the effects of P9.2 on circularization. The circRNA precursor (precursor sequence, SEQ ID NO: 7; FIG. 23) based on ribozyme T was generated and purified through the same processes described in Examples 1.1 and 1.2. The backsplicing site was designed inside the IRES (FIG. 6B) of the nucleotide sequence to be circularized (SEQ ID NO: 50). The preparation of circular RNA was carried out following the procedures described in Example 1.3. The circularization products were digested by RNase R as described in Example 1.4.

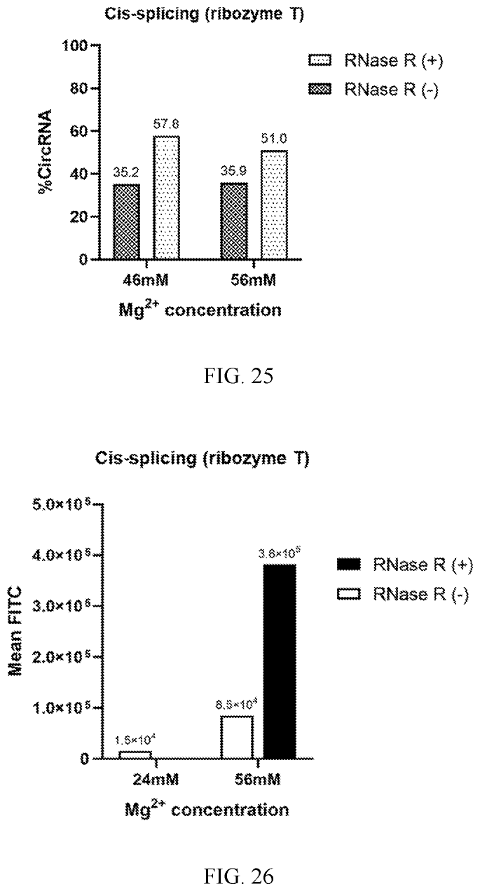

E-Gel shows that after the removal of P9.2, ribozyme T could still catalyze the self-splicing reaction (FIG. 24, tested Mg²⁺ concentrations were 46 mM and 56 mM). The splicing products were subjected to RNase R digestion to confirm the generation of circular RNA (FIG. 24). FA analysis shows that Mg²⁺ with a concentration of about 46 mM could effectively promote circularization (FIG. 25). The expression of reaction products in cells was detected according to the method described in Example 1.6. The expression was enhanced in cells with improved circRNA purity after digestion by RNase R (FIG. 26). These results indicate that ribozyme T without P9.2 could also successfully mediate RNA circularization. It is worth noting that the circularization efficiency of the ribozyme T containing P9.2 (FIGS. 8A and 8B) is higher than that of the ribozyme T without P9.2 (FIGS. 24 and 25).

### Example 7. P1 Duplex Using C-A Wobble Base Pair is Also Compatible with Ribozyme T-Mediated Circularization

Previous studies have found that other wobble base pairs, except for U-G, can also effectively promote the splicing reaction of ribozymes, although the reaction efficiency varies (Dana A. B. et al., Molecular Recognition in a Trans Excision-Splicing Ribozyme: Non-Watson-Crick Base Pairs at the 5′ Splice Site and ωG at the 3′ Splice Site Can Play a Role in Determining the Binding Register of Reaction Substrates, Biochemistry 2005 44 (3), 1067-1077). In this study, the effect of the C-A wobble base pair on circularization was investigated. A circRNA precursor (precursor sequence, SEQ ID NO. 8; FIG. 27) based on ribozyme T was generated and purified through the same processes described in Examples 1.1 and 1.2. The backsplicing site was designed inside the IRES (FIG. 6B) of the nucleotide sequence to be circularized (SEQ ID NO: 50). The preparation of circular RNA was carried out following the procedures described in Example 1.3. The circularization products were digested by RNase R as described in Example 1.4.

E-gel shows that P1 using C-A base pair could still be compatible with the self-splicing reaction of ribozyme T (tested Mg²⁺ concentrations were from 36 mM to 56 mM), and the splicing products were subjected to RNase R digestion to confirm the generation of circular RNA (FIG. 28). FA analysis shows that Mg²⁺ with a concentration of 36 mM to 56 mM could effectively promote circularization (FIG. 29).

### Example 8. Circularization of Precursors by Ribozyme T without Homology Arms and P9.2 Duplex Assistance

The presence of R1 and R2 is crucial for the efficient formation of P9.0, enabling ribozyme T to mediate the complete splicing reactions. The spatial formation of homology arms from R1 and R2, along with P9.2, in a stem-like structure facilitates the proximity of the precursor's termini, thereby promoting splicing. To further investigate the necessity of homology arm sequences and P9.2, they were removed from SEQ ID NO: 1 to generate a circRNA precursor comprising the sequence of SEQ ID NO: 9. In SEQ ID NO: 9, R2 comprises the sequence from the 5′ half of P9.0 to the sequence before the 5′ half of P9.2, and R1 comprises the sequence from the end of P9.2 to ωG.

The circRNA precursor (SEQ ID NO: 9; FIG. 30) based on ribozyme T was generated and purified through the same processes described in Examples 1.1 and 1.2. The backsplicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

Surprisingly, the resulting precursor lacking homology arms and P9.2 still exhibited self-splicing activity, as evidenced by E-Gel analysis, and the digestion by RNase R further confirmed the generation of circular RNA (FIG. 31). Consequently, these results indicate that homology arms and P9.2 are not essential for circularization. However, they may facilitate the proximity of the precursor termini and enhance reaction efficiency.

### Example 9. 5′ and 3′ Homology Arm Sequences are Essential for Higher Circularization Efficiency

Based on the results of Examples 1 (SEQ ID NO: 1), 6 (SEQ ID NO: 7) and 8 (SEQ ID NO: 9), we hypothesize that the formation of a double-stranded region through the 5′ and 3′ homology arm sequences between R1 and R2 in proximity of the ωG is essential for a higher circularization efficiency. To test this hypothesis, the sequence connecting the 3′ half of P9.2 and the 3′ half of P9.0 (i.e., Spacer 1), and the sequence connecting the 5′ half of P9.0 and the 5′ half of P9.2 (i.e., Spacer 2) were removed from SEQ ID NO: 1 to generate a circRNA precursor comprising the sequence of SEQ ID NO: 10. In the absence of Spacer 1 and Spacer 2, the sequence for 3′ half of P9.2 and the sequence for 5′ half of P9.2 in SEQ ID NO: 10 can be simply regarded as 5′ and 3′ homology arm sequences, respectively.

The circRNA precursor (SEQ ID NO: 10; FIG. 32) based on ribozyme T was generated and purified through the same processes described in Examples 1.1 and 1.2. The backsplicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

Results from E-Gel revealed that precursor molecules lacking both spacers can still undergo self-splicing circularization and RNase R digestion effectively enriched circular RNA (FIG. 33). The expression of reaction products in cells was detected according to the method described in Example 1.6. The results show that a precursor lacking both spacers could also successfully circularize through self-splicing, and the expression was enhanced in cells with improved circRNA purity after digestion enrichment by RNase R (FIG. 34). These findings suggest that recognition of the 3′ splice sites can be achieved solely through homologous arms, resembling the design of ribozyme P. In addition, compared to the circularization efficiency of the circRNA precursor comprising the sequence of SEQ ID NO: 7 (FIG. 25, 35.2%), the circRNA precursor comprising the sequence of SEQ ID NO: 10 exhibits a higher circularization efficiency (FIG. 33, 54.9%), indicating that base pairing between R1 and R2 to form a double-stranded region in close proximity of ωG is essential for a higher circularization efficiency. In comparison to the splicing efficiency of the circRNA precursor comprising the sequence of SEQ ID NO: 1 (FIG. 8B, 67.3%), the circRNA precursor comprising the sequence of SEQ ID NO: 10 exhibited a lower splicing efficiency (FIG. 33, 54.9%). However, E-Gel analysis indicated that the precursor comprising the sequence of SEQ ID NO: 1 generated more nicked RNAs in the circularization reaction (FIG. 8A). Therefore, it is plausible that the proportion of circRNA without nicking produced by the precursor comprising the sequence of SEQ ID NO: 1 (FIG. 8A) may be comparable to that of the precursor comprising the sequence of SEQ ID NO: 10 (FIG. 33). This can be easily verified through quantification of the circRNAs and nicked RNAs in the circularized samples using Capillary Quantitative Analysis (PA800 Plus, SCIEX) as described in Example 1.3.

### Example 10. Homology Arm-Independent 3′ Splice Site Recognition in R1 and R2 (Ribozyme P as an Example)

Based on the previous results (for example, Example 9), the 5′ and 3′ homology arm sequences are essential for high efficiency recognition and splicing of the 3′ splice site. To further confirm the necessity of the 5′ and 3′ homology arm sequences, they were directly removed from SEQ ID NO: 6, leaving only two bases for pairing similar to P9.0 (FIG. 35), to generate a circRNA precursor comprising the sequence of SEQ ID NO: 11. The circRNA precursor (SEQ ID NO: 11) was generated and purified through the same processes described in Examples 1.1 and 1.2. The backsplicing site was designed inside the IRES (FIG. 6B) of the nucleotide sequence to be circularized (SEQ ID NO: 50). The preparation of circular RNA was carried out following the procedures described in Example 1.3. The circularization products were digested by RNase R as described in Example 1.4.

E-Gel results demonstrate that the precursor could undergo self-splicing and circularization without the external homology arms (FIG. 36). However, the circularization efficiency (not shown) was lower than when the homology arm sequences were present (FIG. 21). These findings support the idea that homology arm sequences enhance the efficiency of circularization.

### Example 11. Homology Arm-Independent 3′ Splicing Site Recognition in R1 and R2 (Ribozyme T as an Example)

Based on previous results (e.g., Example 9), the recognition and splicing of the 3′ splicing site can be achieved by partially pairing the two ends of the precursor to form a duplex similar to P9.0. To further confirm the necessity of the 5′ and 3′ homology arm sequences for ribozyme T, they were directly removed from SEQ ID NO: 10, leaving only two bases for pairing similar to P9.0, to generate a circRNA precursor comprising the sequence of SEQ ID NO: 39 (FIG. 37).

The circRNA precursor (SEQ ID NO: 39) was generated and purified through the same processes described in Examples 1.1 and 1.2. The backsplicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

Results from E-Gel demonstrated that the precursor could undergo self-splicing and circularization without the external homology arms (FIG. 38). The cellular expression of GFP was enhanced with improved circRNA purity after digestion enrichment by RNase R (FIG. 39). However, the circularization efficiency was lower than when auxiliary elements such as homology arms were present (FIG. 33). These findings support the idea that homology arm sequences enhance the efficiency of circularization.

### Example 12. Inhibition of Circularization in the Absence of Paired Structure Formation Between R1 and R2 (Ribozyme T as an Example)

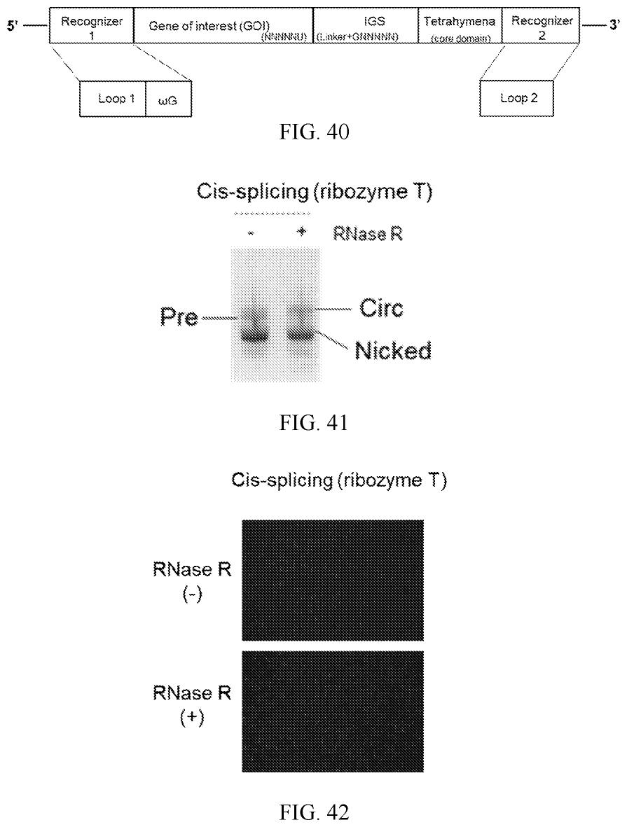

To further validate the significance of the paired structure formed between the 5′ and 3′ ends of the precursor, R1 and R2 were designed to form unpaired structures, like loops, to generate a circRNA precursor comprising the sequence of SEQ ID NO: 40 (FIG. 40).

The circRNA precursor (SEQ ID NO: 40) was generated and purified through the same processes described in Examples 1.1 and 1.2. The back-splicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

The results demonstrated that forming a paired structure between the two ends of the precursor molecule is crucial for completing the two-step self-splicing reaction. Without this paired structure, the circularization efficiency was significantly reduced (FIG. 41). RNase R could not enrich the final product, leading to ineffective expression of GFP in cells (FIG. 42). These findings emphasize the indispensable role of paired structure formation for achieving high circularization efficiency and successful expression of the target protein.

### Example 13. Restoration of Circularization Through Reintroducing Paired Structures in R1 and R2 (Ribozyme T as an Example)

Based on the results obtained in Example 12, it was observed that absence of a paired structure between R1 and R2 led to a significant inhibition of the circularization reaction. To further validate the necessity of paired structure formation at both ends of the precursor, homology arms were reintroduced to generate a circRNA precursor comprising the sequence of SEQ ID NO: 41 (FIG. 43).

The circRNA precursor (SEQ ID NO: 41) was generated and purified through the same processes described in Examples 1.1 and 1.2. The backsplicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

E-Gel results demonstrated that reintroduction of this paired structure resulted in a substantial improvement in circularization efficiency (FIG. 44). Additionally, the circular RNA could be enriched by RNase R, leading to enhanced cellular expression of GFP (FIG. 45). These findings highlight the critical role of the paired structure within both terminals of precursor in promoting efficient circularization. In addition, comparing to the circularization efficiency of the circRNA precursor comprising the sequence of SEQ ID NO: 7 (FIG. 25, 35.2%), the circRNA precursor comprising the sequence of SEQ ID NO: 10 exhibits a higher circularization efficiency (FIG. 33, 54.9%), indicating that base pairing between R1 and R2 to form a double-stranded region in close proximity of ωG is essential for a higher circularization efficiency.

### Example 14. The P9.0 Duplex Functions in Circularization at the Structural Rather than Sequence Level (Ribozyme T as an Example)

Examples 12 and 13 have demonstrated that incorporating complementary pairing structures between R1 and R2 is crucial for effective circularization. To further investigate the flexibility of pairing design, the base pairs within the P9.0 duplex at the 5′ and 3′ positions were swapped while still maintaining the complementary design. The resultant circRNA precursor has the sequence of SEQ ID NO: 44 (FIG. 46).

The circRNA precursor (SEQ ID NO: 44) based on ribozyme T was generated and purified through the same processes described in Examples 1.1 and 1.2. The back-splicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

The results indicated that there were no restrictions on the order (from 5′ to 3′) of base pairs on the structure of the P9.0 duplex to complete the splicing reaction (FIG. 47), and self-circularization of the precursor occurs as long as the structural requirement of a duplex formed in close proximity of ωG is met. Additionally, the circular RNA products were enriched by RNase R, resulting in an increase in protein expression levels in cells (FIG. 48).

### Example 15. The Design of the Cis-Circularization System is Compatible with Other Group I Introns, Such as Anabaena Group I Intron

This study applied the cis-splicing circularization system proposed in the present invention to other group I introns, specifically Anabeana pre-tRNA group I intron.

The circRNA precursor (based on Anabaena (sp. strain PCC 7120)-hereafter referred to as “ribozyme A”) was generated and purified through the same processes described in Examples 1.1 and 1.2 (SEQ ID NO: 45) (FIG. 49). The nucleotide sequence to be circularized (SEQ ID NO: 51) comprises a 5′ UTR comprising an IRES from Enterovirus B, an ORF sequence encoding firefly luciferase (Fluc) and a 3′ UTR. The back-splicing site was designed in the 3′ UTR. Specifically, the target site having a sequence of ‘CTT’ (‘nnu’; corresponding to the upstream exon fragment of the native Anabaena group I intron) and a sequence of ‘AAAA’ (corresponding to the downstream exon fragment of the native Anabaena group I intron) were designed in the 3′ UTR of SEQ ID NO: 51. The GOI was formed by placing the sequence ‘AAAA’ and its downstream sequence in SEQ ID NO: 51 at the 5′ end and the remaining sequence in SEQ ID NO: 51 at the 3′ end. R1 and R2 were designed to include homology arm sequences, spacers and the sequences for P9.0 duplex (see FIG. 49). The preparation of circular RNA was carried out following the procedures described in Example 1.3. The circularization products were digested by RNase R as described in Example 1.4.

E-gel results demonstrated that such a design included only homology arms sequences in R1 and R2, along with the P9.0 duplex (see FIG. 49), which already met the requirements for the splicing reaction (see FIG. 50).

To examine the cellular expression of the circularized RNA, RNase R-treated circularization samples were transfected into A549 cells. A mock transfection served as a negative control. At 24 hours after transfection, the cells grown in 100 μl of culture medium were added with 100 μl of reagent (ωNE-Glo™ Luciferase Assay, Promega) (for 96-well plates) and lysed by rocking and pipetting for roughly 3 minutes at room temperature. Then, the plate was read on a TECAN M1000 Infinite Pro microplate reader using i-control 1.10 software with an integration time of 1,000 ms. The results demonstrated that the resulting circular RNA was enriched by RNase R, leading to increased expression of the reporter gene (luciferase) in cells (FIG. 51).

In this Example, the natural exon sequence flanking the ribozyme A was deleted and replaced with the sequence from within the GOI (e.g., the sequence after ωG, see SEQ ID 45), although the replaced sequence may be partially homology with the natural exon sequence. For ribozyme A, the product of the cis-splicing reaction needed to be further enriched by RNase R so that the band corresponding to circRNA was able to be detected more prominently in the E-Gel (FIG. 50). This result indicates that for certain group I introns (such as ribozyme A in this case), while the natural exon sequence is not essential, it may have specific interactions with the intron region and be involved in the splicing process. Therefore, replacing the complete exon with a homologous sequence from the GOI region is possible, which may optimize the reaction efficiency while avoiding the introduction of foreign sequences.

### Example 16. P10 Duplex and P1 Extension Facilitate Circularization but are Dispensable

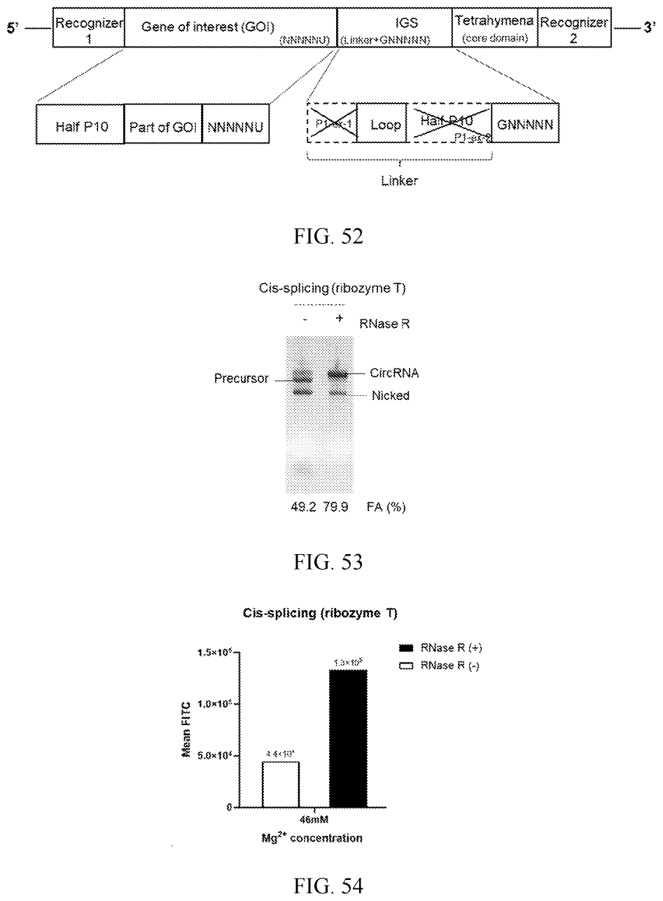

It is reported that the P10 duplex is formed following the initial step of the splicing reaction and is closely associated with the subsequent step of splicing in the self excision of some group I intron, including ribozyme T. To study the roles of P10 duplex and P1 extension in the cis-circularization of the RNA construct of the present application, the entire sequences for P10-2 (including P1-ex-2) as well as P1-ex-1 were removed from the linker sequence (i.e., ‘CACAUUUUACA’) (see the linker sequence in SEQ ID NO: 10) to generate a circRNA precursor comprising the nucleotide sequence of SEQ ID NO: 46 (FIG. 52). This design resulted in a loop sequence constituting the linker sequence between the target site and the IGS. The circRNA precursor (SEQ ID NO:46) based on ribozyme T was generated and purified through the same processes described in Examples 1.1 and 1.2. The back-splicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

E-Gel results demonstrated that the complete removal of the P10 duplex and P1 extension did not prevent the precursor from undergoing the two-step splicing reaction and circularization (FIG. 53). However, the circularization efficiency was notably reduced (FIG. 53, 49.2% with more nicked RNA and less circular RNA) compared to a design containing a P10 duplex and a P1 extension (FIG. 33 in Example 9, 54.9% with less nicked RNA and more circular RNA). Despite a reduced circularization, the resulting circular RNA could be enriched by RNase R, leading to increased expression of the reporter gene (GFP) in cells (FIG. 54). These results suggested that while a P10 duplex and a P1 extension are not required for circularization, they significantly enhance circularization efficiency.

### Example 17. Higher Circularization Efficiency could be Achieved with a Shorter P10 Duplex and a Shorter P1 Extension

To further study the role of P10 duplex and P1 extension, the 5′ end portion of P10-2 and P1-ex-1 were removed from the linker sequence (‘CACAUUUUACAAUG’) (see the linker sequence in SEQ ID NO: 10) to generate a circRNA precursor comprising the nucleotide sequence of SEQ ID NO: 47 (FIG. 55). This design resulted in formation of a shorter P1 extension between the 5′ end ‘CA’ and 3′ end ‘UG’ in the linker sequence and a shorter P10 duplex between the 3-nucletoide sequence ‘CAU’ at 5′ end of the GOI and the 3′ end ‘AUG’ in the linker sequence. The circRNA precursor (SEQ ID NO: 47) based on ribozyme T was generated and purified through the same processes described in Examples 1.1 and 1.2. The back-splicing site was designed inside the ORF (FIG. 6A) of the nucleotide sequence to be circularized (SEQ ID NO: 50). The preparation of circular RNA was carried out following the procedures described in Example 1.3. The circularization products were digested by RNase R as described in Example 1.4.

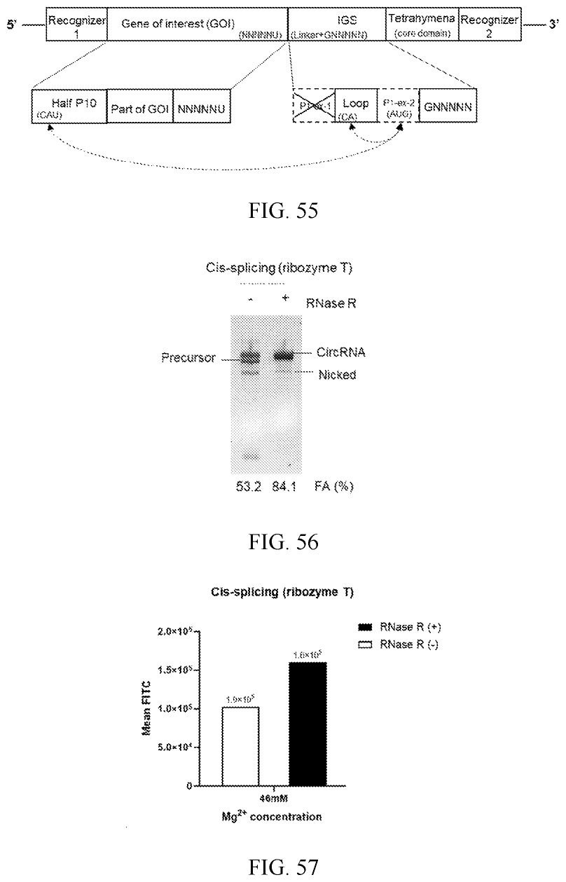

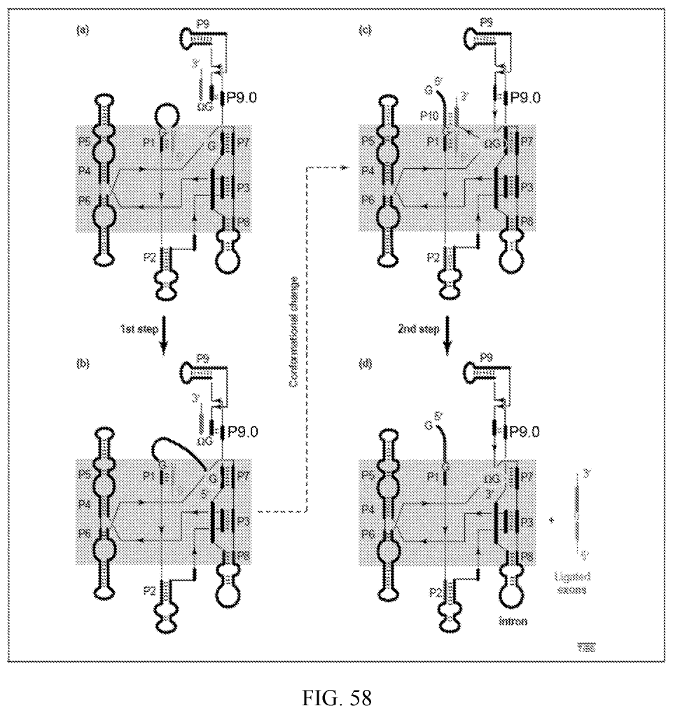

E-Gel results demonstrate that even a shorter P10 duplex and P1 extension significantly improved two-step splicing efficiency (FIG. 56, 53.2% with more circular RNA and less nicked RNA) compared to a version without a P10 duplex and P1 extension (FIG. 53 in Example 16, 49.2% with more nicked RNA and less circular RNA). Additionally, the circular RNA products were enriched by RNase R, resulting in an increase in protein expression levels in cells (FIG. 57).

While the disclosure has been described with respect to specific examples including presently preferred modes of carrying out the disclosure, those skilled in the art will appreciate that there are numerous variations and permutations of the above described systems and techniques. It is to be understood that other embodiments may be utilized and structural and functional modifications may be made without departing from the scope of the present disclosure. Thus, the scope of the disclosure should be construed broadly as set forth in the appended claims.

Sequencing List

| | |
|---|---|
| SEQ ID NO: 1 | ACCGUCGAUUGUCCACUGGUC [symbol] CAU |
| [R1: Arm I- [symbol] - | GGCCGACAAGCAGAAGAACGGCAUCAAGGCGAACUUCAAGAUCC |
| Spacer 1- [symbol] - [symbol] | GCCACAACAUCGAGGACGGCAGCGUGCAGCUCGCCGACCACUAC |
| GOI with P10-1 at the 5′ end and | CAGCAGAACACCCCCAUCGGCGACGGCCCCGUGCUGCUGCCCGAC |
| a target site at the 3′ end; Linker | AACCACUACCUGAGCACCCAGUCCGCCCUGAGCAAAGACCCCAA |
| sequence: P1-ex-1-Loop | CGAGAAGCGCGAUCACAUGGUCCUGCUGGAGUUCGUGACCGCCG |
| sequence (including 5' portion of | CCGGGAUCACUCUCGGCAUGGACGAGCUGUACAAGUGAUAAACC |
| P10-2)-P1-ex-2 (3′ portion of | GGUGCUGGAGCCUCGGUGGCCAUGCUUCUUGCCCCUUGGGCCUC |
| P10-2); [symbol] ; Tetrahymena | CCCCCAGCCCCUCCUCCCCUUCCUGCACCCGUACCCCCGUGGUCU |
| ribozyme core: SEQ ID NO: 17; | UUGAAUAAAGUCUGAGUGGGCGGCCUCGAGAAAAAAAAAAAAC |
| R2: [symbol] -Spacer 2- | AAAAAAAAAAAACCAAAAAAAAAAAAUAAAAAACUAAUUAAAA |
| [symbol] -Arm I; Tail] | CAGCGGAUGGGUACCCCACCAUCCGACCCACUGGGUGUAGUACU |
| related to FIG. 6A | CUGGUACUUCGUACCUUUGUACGCCUGUUCUUCCCAUUGUACCC |
| | UUCCUGAACUUCCAACCCAAGUAACGUUAGAAGCUCAACAUUUA |
| | GUACAACAGGAAGCACCACAUCCAGUGGUGUUUAGUACAAGCAC |
| | UUCUGUUUCCCCGGAGCGAGGUAUAGGCUGUACCCACUGCCAAA |
| | AACCUUUAACCGUUAUCCGCCAACCAACUACGUAAAAGCUAGUA |
| | GUAUUAUGUUUUUAACUAGGCGUUCGAUCAGGUGGAUUUCCCCU |
| | CCACUAGUUUGGUCGAUGAGGCUAGGAAUUCCCCACGGGUGACC |
| | GUGUCCUAGCCUGCGUGGCGGCCAACCCAGCUUAUGCUGGGACG |
| | CCUUUUUAUAGACAUGGUGUGAAGACUCGCAUGUGCUUGGUUG |
| | UGAUUCCUCCGGCCCCUGAAUGCGGCUAACCUUAACCCUGGAGC |
| | CUUGUGUCACAAACCAGUGAUGAUAAGGUCGUAAUGAGCAAUUC |
| | CGGGACGGGACCGACUACUUUGGGUGUCCGUGUUUCUUAUUUUU |
| | CUUAUUAUUGUCUUAUGGUCACAGCAUAUAUAUAACAUAUACU |
| | GUGAUCAUGGUGAGCAAGGGCGAGGAGCUGUUCACCGGGGUGG |
| | UGCCCAUCCUGGUCGAGCUGGACGGCGACGUAAACGGCCACAAG |
| | UUCAGCGUGUCUGGCGAGGGCGAGGGCGAUGCCACCUACGGCAA |
| | GCUGACCCUGAAGUUCAUCUGCACCACCGGCAAGCUGCCCGUGC |
| | CCUGGCCCACCCUCGUGACCACCCUGACCUACGGCGUGCAGUGC |
| | UUCAGCCGCUACCCCGACCACAUGAAGCAGCACGACUUCUUCAA |
| | GUCCGCCAUGCCCGAAGGCUACGUCCAGGAGCGCACCAUCUUCU |
| | UCAAGGACGACGGCAACUACAAGACCCGCGCCGAGGUGAAGUUC |
| | GAGGGCGACACCCUGGUGAACCGCAUCGAGCUGAAGGGCAUCGA |
| | CUUCAAGGAGGACGGCAACAUCCUGGGGCACAAGCUGGAGUACA |
| | ACUACAACAGCCACAACGUCUAUAUCAUCACAUUUUACAGGCCA |
| | [symbol] AAAAGUUAUCAGGCAUGCACCUGGUAGCUAGUCU |
| | UUAAACCAAUAGAUUGCAUCGGUUUAAAAGGCAAGACCGUCA |
| | AAUUGCGGGAAAGGGGUCAACAGCCGUUCAGUACCAAGUCUC |
| | AGGGGAAACUUUGAGAUGGCCUUGCAAAGGGUAUGGUAAUAA |
| | GCUGACGGACAUGGUCCUAACCACGCAGCCAAGUCCUAAGUC |
| | AACAGAUCUUCUGUUGAUAUGGAUGCAGUUCACAGACUAAAU |
| | GUCGGUCGGGGAAGAUGUAUUCUUCUCAUAAGAUAUAGUCG |
| | [symbol] CUCCUUAAUGGGAGCUAGCGGAUGAAGUGAUGCAACAC |
| | UGGAGCCGCUGGGAACUAAUUACCAGUGGACAAUCGACGGAUA |
| | ACAGCAUAUCUAGAAAAAAAAAAAAAAAAAAAAAAAAAAAAAA |
| | AAAAAAAAAAAAAAAAAAAAAAAA |
| SEQ ID NO: 2 | ACCGUCGAUUGUCCACUGGUC [symbol] UUA |
| [R1: Arm I- [symbol] - | UAGACAUGGUGUGAAGACUCGCAUGUGCUUGGUUGUGAUUCCUC |
| Spacer 1- [symbol] - [symbol] ; | CGGCCCCUGAAUGCGGCUAACCUUAACCCUGGAGCCUUGUGUCA |
| GOI with P10-1 at the 5′ end and | CAAACCAGUGAUGAUAAGGUCGUAAUGAGCAAUUCCGGGACGGG |
| a target site at the 3′ end; Linker | ACCGACUACUUUGGGUGUCCGUGUUUCUUAUUUUUCUUAUUAUU |
| sequence: P1-ex-1-Loop | GUCUUAUGGUCACAGCAUAUAUAUAACAUAUACUGUGAUCAUG |
| sequence (including 5' portion of | GUGAGCAAGGGCGAGGAGCUGUUCACCGGGGUGGUGCCCAUCCU |
| P10-2)-P1-ex-2 (3′ portion of | GGUCGAGCUGGACGGCGACGUAAACGGCCACAAGUUCAGCGUGU |
| P10-2); [symbol] ; Tetrahymena | CUGGCGAGGGCGAGGGCGAUGCCACCUACGGCAAGCUGACCCUG |
| ribozyme core: SEQ ID NO: 17; | AAGUUCAUCUGCACCACCGGCAAGCUGCCCGUGCCCUGGCCCAC |
| R2: [symbol] -Spacer 2- | CCUCGUGACCACCCUGACCUACGGCGUGCAGUGCUUCAGCCGCU |
| [symbol] -Arm I; Tail] | ACCCCGACCACAUGAAGCAGCACGACUUCUUCAAGUCCGCCAUG |
| related to FIG. 6B | CCCGAAGGCUACGUCCAGGAGCGCACCAUCUUCUUCAAGGACGA |
| | CGGCAACUACAAGACCCGCGCCGAGGUGAAGUUCGAGGGCGACA |
| | CCCUGGUGAACCGCAUCGAGCUGAAGGGCAUCGACUUCAAGGAG |
| | GACGGCAACAUCCUGGGGCACAAGCUGGAGUACAACUACAACAG |
| | CCACAACGUCUAUAUCAUGGCCGACAAGCAGAAGAACGGCAUCA |
| | AGGCGAACUUCAAGAUCCGCCACAACAUCGAGGACGGCAGCGUG |
| | CAGCUCGCCGACCACUACCAGCAGAACACCCCCAUCGGCGACGGC |
| | CCCGUGCUGCUGCCCGACAACCACUACCUGAGCACCCAGUCCGCC |
| | CUGAGCAAAGACCCCAACGAGAAGCGCGAUCACAUGGUCCUGCU |
| | GGAGUUCGUGACCGCCGCCGGGAUCACUCUCGGCAUGGACGAGC |
| | UGUACAAGUGAUAAACCGGUGCUGGAGCCUCGGUGGCCAUGCUU |
| | CUUGCCCCUUGGGCCUCCCCCCAGCCCCUCCUCCCCUUCCUGCAC |
| | CCGUACCCCCGUGGUCUUUGAAUAAAGUCUGAGUGGGCGGCCUC |
| | GAGAAAAAAAAAAAACAAAAAAAAAAAACCAAAAAAAAAAAAU |
| | AAAAAACUAAUUAAAACAGCGGAUGGGUACCCCACCAUCCGACC |
| | CACUGGGUGUAGUACUCUGGUACUUCGUACCUUUGUACGCCUGU |
| | UCUUCCCAUUGUACCCUUCCUGAACUUCCAACCCAAGUAACGUU |
| | AGAAGCUCAACAUUUAGUACAACAGGAAGCACCACAUCCAGUGG |
| | UGUUUAGUACAAGCACUUCUGUUUCCCCGGAGCGAGGUAUAGGC |
| | UGUACCCACUGCCAAAAACCUUUAACCGUUAUCCGCCAACCAAC |
| | UACGUAAAAGCUAGUAGUAUUAUGUUUUUAACUAGGCGUUCGA |
| | UCAGGUGGAUUUCCCCUCCACUAGUUUGGUCGAUGAGGCUAGGA |
| | AUUCCCCACGGGUGACCGUGUCCUAGCCUGCGUGGCGGCCAACC |
| | CAGCUUAUGCUGGGACGCCUUUUUACACAUUUUACAUCUAU [symbol] |
| | [symbol] AAAAGUUAUCAGGCAUGCACCUGGUAGCUAGUCUUUAA |
| | ACCAAUAGAUUGCAUCGGUUUAAAAGGCAAGACCGUCAAAUU |
| | GCGGGAAAGGGGUCAACAGCCGUUCAGUACCAAGUCUCAGGG |
| | GAAACUUUGAGAUGGCCUUGCAAAGGGUAUGGUAAUAAGCUG |
| | ACGGACAUGGUCCUAACCACGCAGCCAAGUCCUAAGUCAACA |
| | GAUCUUCUGUUGAUAUGGAUGCAGUUCACAGACUAAAUGUCG |
| | GUCGGGGAAGAUGUAUUCUUCUCAUAAGAUAUAGUCGGACCU |
| | CUCCUUAAUGGGAGCUAGCGGAUGAAGUGAUGCAACACUGGA |
| | GCCGCUGGGAACUAAUUACCAGUGGACAAUCGACGGAUAACAGC |
| | AUAUCUAGAAAAAAAAAAAAAAAAAAAAAAAAAAAAAAAAAAA |
| | AAAAAAAAAAAAAAAAAAA |
| SEQ ID NO: 3 | ACCGUCGAUUGUCCACUGGUCGCCUUG [symbol] UUAUAGACAUGGUG |
| [R1: Arm III- [symbol] - | UGAAGACUCGCAUGUGCUUGGUUGUGAUUCCUCCGGCCCCUGAA |
| [symbol] | UGCGGCUAACCUUAACCCUGGAGCCUUGUGUCACAAACCAGUGA |
| GOI with P10-1 at the 5′ end and | UGAUAAGGUCGUAAUGAGCAAUUCCGGGACGGGACCGACUACUU |
| a target site at the 3′ end; Linker | UGGGUGUCCGUGUUUCUUAUUUUUCUUAUUAUUGUCUUAUGGU |
| sequence: P1-ex-1-Loop | CACAGCAUAUAUAUAACAUAUACUGUGAUCAUGGUGAGCAAGG |
| sequence (including 5' portion of | GCGAGGAGCUGUUCACCGGGGUGGUGCCCAUCCUGGUCGAGCUG |
| P10-2)-P1-ex-2 (3′ portion of | GACGGCGACGUAAACGGCCACAAGUUCAGCGUGUCUGGCGAGGG |
| P10-2); [symbol] ; Pneumocystis | CGAGGGCGAUGCCACCUACGGCAAGCUGACCCUGAAGUUCAUCU |
| carinii ribozyme core: SEQ ID | GCACCACCGGCAAGCUGCCCGUGCCCUGGCCCACCCUCGUGACCA |
| NO: 19; | CCCUGACCUACGGCGUGCAGUGCUUCAGCCGCUACCCCGACCAC |
| R2: [symbol] -Arm III; | AUGAAGCAGCACGACUUCUUCAAGUCCGCCAUGCCCGAAGGCUA |
| Tail] | CGUCCAGGAGCGCACCAUCUUCUUCAAGGACGACGGCAACUACA |
| related to FIG. 2 and 10 | AGACCCGCGCCGAGGUGAAGUUCGAGGGCGACACCCUGGUGAAC |
| | CGCAUCGAGCUGAAGGGCAUCGACUUCAAGGAGGACGGCAACAU |
| | CCUGGGGCACAAGCUGGAGUACAACUACAACAGCCACAACGUCU |
| | AUAUCAUGGCCGACAAGCAGAAGAACGGCAUCAAGGCGAACUUC |
| | AAGAUCCGCCACAACAUCGAGGACGGCAGCGUGCAGCUCGCCGA |
| | CCACUACCAGCAGAACACCCCCAUCGGCGACGGCCCCGUGCUGCU |
| | GCCCGACAACCACUACCUGAGCACCCAGUCCGCCCUGAGCAAAG |
| | ACCCCAACGAGAAGCGCGAUCACAUGGUCCUGCUGGAGUUCGUG |
| | ACCGCCGCCGGGAUCACUCUCGGCAUGGACGAGCUGUACAAGUG |
| | AUAAACCGGUGCUGGAGCCUCGGUGGCCAUGCUUCUUGCCCCUU |
| | GGGCCUCCCCCCAGCCCCUCCUCCCCUUCCUGCACCCGUACCCCC |
| | GUGGUCUUUGAAUAAAGUCUGAGUGGGGGCCUCGAGAAAAAA |
| | AAAAAACAAAAAAAAAAAACCAAAAAAAAAAAAUAAAAAACUA |
| | AUUAAAACAGCGGAUGGGUACCCCACCAUCCGACCCACUGGGUG |
| | UAGUACUCUGGUACUUCGUACCUUUGUACGCCUGUUCUUCCCAU |
| | UGUACCCUUCCUGAACUUCCAACCCAAGUAACGUUAGAAGCUCA |
| | ACAUUUAGUACAACAGGAAGCACCACAUCCAGUGGUGUUUAGUA |
| | CAAGCACUUCUGUUUCCCCGGAGCGAGGUAUAGGCUGUACCCAC |
| | UGCCAAAAACCUUUAACCGUUAUCCGCCAACCAACUACGUAAAA |
| | GCUAGUAGUAUUAUGUUUUUAACUAGGCGUUCGAUCAGGUGGA |
| | UUUCCCCUCCACUAGUUUGGUCGAUGAGGCUAGGAAUUCCCCAC |
| | GGGUGACCGUGUCCUAGCCUGCGUGGCGGCCAACCCAGCUUAUG |
| | CUGGGACGCCUUUUACCUUCUAUA [symbol] GAAAGCGGCGUG |
| | AAAACGUUAGCUAGUGAUCUGGAAUAAAUUCAGAUUGCGACA |
| | CUGUCAAAUUGCGGGGAAGCCCUAAAUAUUCAACUACUAAGC |
| | AGUUUGUGGAAACACAGCUGUGGCCGAGUUAAUAGCCCUGGG |
| | UAUAGUAACAAUGUUGAAUAUGAAUCUUUUGGGAGAUGAAAU |
| | GGGUGAUCCGCAGCCAAGUCCUAAGGGCAUUUUUGUCUAUGG |
| | AUGCAGUUCAACGACUAGAUGGCAGUGGGUAUUGUAAGGAAU |
| | UGCAGUUUUCUUGCAGUGCUUAAGGUAUAGUCU [symbol] |
| | CAGUGGACAAUCGACGGAUAACAGCAUAUCUAGAAAAAAAAAA |
| | AAAAAAAAAAAAAAAAAAAAAAAAAAAAAAAAAAAAAAAAA |
| | AA |
| SEQ ID NO: 4 | ACCGUCGAUUGUCCACUGGUCUUACAUCUAUA [symbol] AAAA |
| [Arm I; | GUUAUCAGGCAUGCACCUGGUAGCUAGUCUUUAAACCAAUAG |
| Linker sequence including 5′ | AUUGCAUCGGUUUAAAAGGCAAGACCGUCAAAUUGCGGGAAA |
| portion of P10-2 and P1-ex-1 (3′ | GGGGUCAACAGCCGUUCAGUACCAAGUCUCAGGGGAAACUUU |
| portion of P10-2); [symbol] ; | GAGAUGGCCUUGCAAAGGGUAUGGUAAUAAGCUGACGGACAU |
| Tetrahymena ribozyme core: | GGUCCUAACCACGCAGCCAAGUCCUAAGUCAACAGAUCUUCU |
| from IGS end to ωG, SEQ ID | GUUGAUAUGGAUGCAGUUCACAGACUAAAUGUCGGUCGGGGA |
| NO: 18); GOI with P10-1 at the | AGAUGUAUUCUUCUCAUAAGAUAUAGUCGGACCUCUCCUUAA |
| 5′ end and a target site at the 3′ | UGGGAGCUAGCGGAUGAAGUGAUGCAACACUGGAGCCGCUGG |
| end; Linker sequence including | GAACUAAUUUGUAUGCGAAAGUAUAUUGAUUAGUUUUGGAGU |
| P1-ex-2; | ACUCGUUAUAGACAUGGUGUGAAGACUCGCAUGUGCUUGGUUGU |
| Arm I; Tail] | GAUUCCUCCGGCCCCUGAAUGCGGCUAACCUUAACCCUGGAGCC |
| related to FIG. 13 | UUGUGUCACAAACCAGUGAUGAUAAGGUCGUAAUGAGCAAUUCC |
| | GGGACGGGACCGACUACUUUGGGUGUCCGUGUUUCUUAUUUUUC |
| | UUAUUAUUGUCUUAUGGUCACAGCAUAUAUAUAACAUAUACUG |
| | UGAUCAUGGUGAGCAAGGGCGAGGAGCUGUUCACCGGGGUGGU |
| | GCCCAUCCUGGUCGAGCUGGACGGCGACGUAAACGGCCACAAGU |
| | UCAGCGUGUCUGGCGAGGGCGAGGGCGAUGCCACCUACGGCAAG |
| | CUGACCCUGAAGUUCAUCUGCACCACCGGCAAGCUGCCCGUGCC |
| | CUGGCCCACCCUCGUGACCACCCUGACCUACGGCGUGCAGUGCU |
| | UCAGCCGCUACCCCGACCACAUGAAGCAGCACGACUUCUUCAAG |
| | UCCGCCAUGCCCGAAGGCUACGUCCAGGAGCGCACCAUCUUCUU |
| | CAAGGACGACGGCAACUACAAGACCCGCGCCGAGGUGAAGUUCG |
| | AGGGCGACACCCUGGUGAACCGCAUCGAGCUGAAGGGCAUCGAC |
| | UUCAAGGAGGACGGCAACAUCCUGGGGCACAAGCUGGAGUACAA |
| | CUACAACAGCCACAACGUCUAUAUCAUGGCCGACAAGCAGAAGA |
| | ACGGCAUCAAGGCGAACUUCAAGAUCCGCCACAACAUCGAGGAC |
| | GGCAGCGUGCAGCUCGCCGACCACUACCAGCAGAACACCCCCAU |
| | CGGCGACGGCCCCGUGCUGCUGCCCGACAACCACUACCUGAGCA |
| | CCCAGUCCGCCCUGAGCAAAGACCCCAACGAGAAGCGCGAUCAC |
| | AUGGUCCUGCUGGAGUUCGUGACCGCCGCCGGGAUCACUCUCGG |
| | CAUGGACGAGCUGUACAAGUGAUAAACCGGUGCUGGAGCCUCGG |
| | UGGCCAUGCUUCUUGCCCCUUGGGCCUCCCCCCAGCCCCUCCUCC |
| | CCUUCCUGCACCCGUACCCCCGUGGUCUUUGAAUAAAGUCUGAG |
| | UGGGCGGCCUCGAGAAAAAAAAAAAACAAAAAAAAAAAACCAA |
| | AAAAAAAAAAUAAAAAACUAAUUAAAACAGCGGAUGGGUACCC |
| | CACCAUCCGACCCACUGGGUGUAGUACUCUGGUACUUCGUACCU |
| | UUGUACGCCUGUUCUUCCCAUUGUACCCUUCCUGAACUUCCAAC |
| | CCAAGUAACGUUAGAAGCUCAACAUUUAGUACAACAGGAAGCAC |
| | CACAUCCAGUGGUGUUUAGUACAAGCACUUCUGUUUCCCCGGAG |
| | CGAGGUAUAGGCUGUACCCACUGCCAAAAACCUUUAACCGUUAU |
| | CCGCCAACCAACUACGUAAAAGCUAGUAGUAUUAUGUUUUUAAC |
| | UAGGCGUUCGAUCAGGUGGAUUUCCCCUCCACUAGUUUGGUCGA |
| | UGAGGCUAGGAAUUCCCCACGGGUGACCGUGUCCUAGCCUGCGU |
| | GGCGGCCAACCCAGCUUAUGCUGGGACGCCUUUUUACACAUUAC |
| | CAGUGGACAAUCGACGGAUAACAGCAUAUCUAGAAAAAAAAAA |
| | AAAAAAAAAAAAAAAAAAAAAAAAAAAAAAAAAAAAAAAAAAA |
| | A |
| SEQ ID NO: 5 | GGACGGGCAAGUUACAUCUAUAA [symbol] AAAAGUUAUCAGGC |
| [Arm II; | AUGCACCUGGUAGCUAGUCUUUAAACCAAUAGAUUGCAUCGG |
| Linker sequence including | UUUAAAAGGCAAGACCGUCAAAUUGCGGGAAAGGGGUCAACA |
| 5' portion of P10-2 and P1-ex-1 (3′ | GCCGUUCAGUACCAAGUCUCAGGGGAAACUUUGAGAUGGCCU |
| portion of P10-2); [symbol] ; | UGCAAAGGGUAUGGUAAUAAGCUGACGGACAUGGUCCUAACC |
| Tetrahymena ribozyme core: | ACGCAGCCAAGUCCUAAGUCAACAGAUCUUCUGUUGAUAUGG |
| from IGS end to ωG SEQ ID | AUGCAGUUCACAGACUAAAUGUCGGUCGGGGAAGAUGUAUUC |
| NO: 18); GOI with P10-1 at the | UUCUCAUAAGAUAUAGUCGGACCUCUCCUUAAUGGGAGCUAG |
| 5′ end and a target site at the 3′ | CGGAUGAAGUGAUGCAACACUGGAGCCGCUGGGAACUAAUUU |
| end; Linker sequence including | GUAUGCGAAAGUAUAUUGAUUAGUUUUGGAGUACUCGUUAUA |
| P1-ex-2; | GACAUGGUGUGAAGACUCGCAUGUGCUUGGUUGUGAUUCCUCCG |
| Arm II; Tail] | GCCCCUGAAUGCGGCUAACCUUAACCCUGGAGCCUUGUGUCACA |
| related to FIG. 13 | AACCAGUGAUGAUAAGGUCGUAAUGAGCAAUUCCGGGACGGGAC |
| | CGACUACUUUGGGUGUCCGUGUUUCUUAUUUUUCUUAUUAUUG |
| | UCUUAUGGUCACAGCAUAUAUAUAACAUAUACUGUGAUCAUGG |
| | UGAGCAAGGGCGAGGAGCUGUUCACCGGGGUGGUGCCCAUCCUG |
| | GUCGAGCUGGACGGCGACGUAAACGGCCACAAGUUCAGCGUGUC |
| | UGGCGAGGGCGAGGGCGAUGCCACCUACGGCAAGCUGACCCUGA |
| | AGUUCAUCUGCACCACCGGCAAGCUGCCCGUGCCCUGGCCCACCC |
| | UCGUGACCACCCUGACCUACGGCGUGCAGUGCUUCAGCCGCUAC |
| | CCCGACCACAUGAAGCAGCACGACUUCUUCAAGUCCGCCAUGCC |
| | CGAAGGCUACGUCCAGGAGCGCACCAUCUUCUUCAAGGACGACG |
| | GCAACUACAAGACCCGCGCCGAGGUGAAGUUCGAGGGCGACACC |
| | CUGGUGAACCGCAUCGAGCUGAAGGGCAUCGACUUCAAGGAGGA |
| | CGGCAACAUCCUGGGGCACAAGCUGGAGUACAACUACAACAGCC |
| | ACAACGUCUAUAUCAUGGCCGACAAGCAGAAGAACGGCAUCAAG |
| | GCGAACUUCAAGAUCCGCCACAACAUCGAGGACGGCAGCGUGCA |
| | GCUCGCCGACCACUACCAGCAGAACACCCCCAUCGGCGACGGCCC |
| | CGUGCUGCUGCCCGACAACCACUACCUGAGCACCCAGUCCGCCCU |
| | GAGCAAAGACCCCAACGAGAAGCGCGAUCACAUGGUCCUGCUGG |
| | AGUUCGUGACCGCCGCCGGGAUCACUCUCGGCAUGGACGAGCUG |
| | UACAAGUGAUAAACCGGUGCUGGAGCCUCGGUGGCCAUGCUUCU |
| | UGCCCCUUGGGCCUCCCCCCAGCCCCUCCUCCCCUUCCUGCACCC |
| | GUACCCCCGUGGUCUUUGAAUAAAGUCUGAGUGGGCGGCCUCGA |
| | GUUAAAACAGCGGAUGGGUACCCCACCAUCCGACCCACUGGGUG |
| | UAGUACUCUGGUACUUCGUACCUUUGUACGCCUGUUCUUCCCAU |
| | UGUACCCUUCCUGAACUUCCAACCCAAGUAACGUUAGAAGCUCA |
| | ACAUUUAGUACAACAGGAAGCACCACAUCCAGUGGUGUUUAGUA |
| | CAAGCACUUCUGUUUCCCCGGAGCGAGGUAUAGGCUGUACCCAC |
| | UGCCAAAAACCUUUAACCGUUAUCCGCCAACCAACUACGUAAAA |
| | GCUAGUAGUAUUAUGUUUUUAACUAGGCGUUCGAUCAGGUGGA |
| | UUUCCCCUCCACUAGUUUGGUCGAUGAGGCUAGGAAUUCCCCAC |
| | GGGUGACCGUGUCCUAGCCUGCGUGGCGGCCAACCCAGCUUAUG |
| | CUGGGACGCCUUUUUACACAUUCUUGCCCGUCCUCUAGAAAAA |
| | AAAAAAAAAAAAAAAAAAAAAAAAAAAAAAAAAAAAAAAAAAA |
| | AAAAAA |
| SEQ ID NO: 6 | ACCGUCGAUUGUCCACUGGUCGCCUUG [symbol] UUAUAGACAUGGUG |
| [R1: Arm III- [symbol] - | UGAAGACUCGCAUGUGCUUGGUUGUGAUUCCUCCGGCCCCUGAA |
| [symbol] | UGCGGCUAACCUUAACCCUGGAGCCUUGUGUCACAAACCAGUGA |
| GOI with P10-1 at the 5′ end a | UGAUAAGGUCGUAAUGAGCAAUUCCGGGACGGGACCGACUACUU |
| target site at the 3′ end; Linker | UGGGUGUCCGUGUUUCUUAUUUUUCUUAUUAUUGUCUUAUGGU |
| sequence: P1-ex-1-Loop | CACAGCAUAUAUAUAACAUAUACUGUGAUCAUGGUGAGCAAGG |
| sequence (including 5′ portion of | GCGAGGAGCUGUUCACCGGGGUGGUGCCCAUCCUGGUCGAGCUG |
| P10-2)-P1-ex-2 (3′ portion of | GACGGCGACGUAAACGGCCACAAGUUCAGCGUGUCUGGCGAGGG |
| P10-2); [symbol] ; Pneumocystis | CGAGGGCGAUGCCACCUACGGCAAGCUGACCCUGAAGUUCAUCU |
| carinii ribozyme core: SEQ ID | GCACCACCGGCAAGCUGCCCGUGCCCUGGCCCACCCUCGUGACCA |
| NO: 19; | CCCUGACCUACGGCGUGCAGUGCUUCAGCCGCUACCCCGACCAC |
| R2: [symbol] -Arm III; | AUGAAGCAGCACGACUUCUUCAAGUCCGCCAUGCCCGAAGGCUA |
| Tail] | CGUCCAGGAGCGCACCAUCUUCUUCAAGGACGACGGCAACUACA |
| related to FIG. 19 | AGACCCGCGCCGAGGUGAAGUUCGAGGGCGACACCCUGGUGAAC |
| | CGCAUCGAGCUGAAGGGCAUCGACUUCAAGGAGGACGGCAACAU |
| | CCUGGGGCACAAGCUGGAGUACAACUACAACAGCCACAACGUCU |
| | AUAUCAUGGCCGACAAGCAGAAGAACGGCAUCAAGGCGAACUUC |
| | AAGAUCCGCCACAACAUCGAGGACGGCAGCGUGCAGCUCGCCGA |
| | CCACUACCAGCAGAACACCCCCAUCGGCGACGGCCCCGUGCUGCU |
| | GCCCGACAACCACUACCUGAGCACCCAGUCCGCCCUGAGCAAAG |
| | ACCCCAACGAGAAGCGCGAUCACAUGGUCCUGCUGGAGUUCGUG |
| | ACCGCCGCCGGGAUCACUCUCGGCAUGGACGAGCUGUACAAGUG |
| | AUAAACCGGUGCUGGAGCCUCGGUGGCCAUGCUUCUUGCCCCUU |
| | GGGCCUCCCCCCAGCCCCUCCUCCCCUUCCUGCACCCGUACCCCC |
| | GUGGUCUUUGAAUAAAGUCUGAGUGGGCGGCCUCGAGAAAAAA |
| | AAAAAACAAAAAAAAAAAACCAAAAAAAAAAAAUAAAAAACUA |
| | AUUAAAACAGCGGAUGGGUACCCCACCAUCCGACCCACUGGGUG |
| | UAGUACUCUGGUACUUCGUACCUUUGUACGCCUGUUCUUCCCAU |
| | UGUACCCUUCCUGAACUUCCAACCCAAGUAACGUUAGAAGCUCA |
| | ACAUUUAGUACAACAGGAAGCACCACAUCCAGUGGUGUUUAGUA |
| | CAAGCACUUCUGUUUCCCCGGAGCGAGGUAUAGGCUGUACCCAC |
| | UGCCAAAAACCUUUAACCGUUAUCCGCCAACCAACUACGUAAAA |
| | GCUAGUAGUAUUAUGUUUUUAACUAGGCGUUCGAUCAGGUGGA |
| | UUUCCCCUCCACUAGUUUGGUCGAUGAGGCUAGGAAUUCCCCAC |
| | GGGUGACCGUGUCCUAGCCUGCGUGGCGGCCAACCCAGCUUAUG |
| | CUGGGACGCCUUUUACCUUCUAUAA [symbol] GAAAGCGGCGUG |
| | AAAACGUUAGCUAGUGAUCUGGAAUAAAUUCAGAUUGCGACA |
| | CUGUCAAAUUGCGGGGAAGCCCUAAAUAUUCAACUACUAAGC |
| | AGUUUGUGGAAACACAGCUGUGGCCGAGUUAAUAGCCCUGGG |
| | UAUAGUAACAAUGUUGAAUAUGAAUCUUUUGGGAGAUGAAAU |
| | GGGUGAUCCGCAGCCAAGUCCUAAGGGCAUUUUUGUCUAUGG |
| | AUGCAGUUCAACGACUAGAUGGCAGUGGGUAUUGUAAGGAAU |
| | UGCAGUUUUCUUGCAGUGCUUAAGGUAUAGUCU [symbol] |
| | CAGUGGACAAUCGACGGAUAACAGCAUAUCUAGAAAAAAAAAA |
| | AAAAAAAAAAAAAAAAAAAAAAAAAAAAAAAAAAAAAAAAAAA |
| | A |
| SEQ ID NO: 7 | ACCGUCGAUUGUCCACUGGUC [symbol] UUAUAGACAUGG |
| [R1: Arm I-Spacer 1- | UGUGAAGACUCGCAUGUGCUUGGUUGUGAUUCCUCCGGCCCCUG |
| [symbol] - [symbol] | AAUGCGGCUAACCUUAACCCUGGAGCCUUGUGUCACAAACCAGU |
| GOI with P10-1 at the 5′ end and | GAUGAUAAGGUCGUAAUGAGCAAUUCCGGGACGGGACCGACUAC |
| a target site at the 3′ end; Linker | UUUGGGUGUCCGUGUUUCUUAUUUUUCUUAUUAUUGUCUUAUG |
| sequence: P1-ex-1-Loop | GUCACAGCAUAUAUAUAACAUAUACUGUGAUCAUGGUGAGCAA |
| sequence (including 5′ portion of | GGGCGAGGAGCUGUUCACCGGGGUGGUGCCCAUCCUGGUCGAGC |
| P10-2)-P1-ex-2 (3′ portion of | UGGACGGCGACGUAAACGGCCACAAGUUCAGCGUGUCUGGCGAG |
| P10-2); [symbol] ; Tetrahymena | GGCGAGGGCGAUGCCACCUACGGCAAGCUGACCCUGAAGUUCAU |
| ribozyme core: SEQ ID NO: 17; | CUGCACCACCGGCAAGCUGCCCGUGCCCUGGCCCACCCUCGUGAC |
| R2: [symbol] -Spacer 2- | CACCCUGACCUACGGCGUGCAGUGCUUCAGCCGCUACCCCGACC |
| Arm I; Tail] | ACAUGAAGCAGCACGACUUCUUCAAGUCCGCCAUGCCCGAAGGC |
| related to FIG. 23 | UACGUCCAGGAGCGCACCAUCUUCUUCAAGGACGACGGCAACUA |
| | CAAGACCCGCGCCGAGGUGAAGUUCGAGGGCGACACCCUGGUGA |
| | ACCGCAUCGAGCUGAAGGGCAUCGACUUCAAGGAGGACGGCAAC |
| | AUCCUGGGGCACAAGCUGGAGUACAACUACAACAGCCACAACGU |
| | CUAUAUCAUGGCCGACAAGCAGAAGAACGGCAUCAAGGCGAACU |
| | UCAAGAUCCGCCACAACAUCGAGGACGGCAGCGUGCAGCUCGCC |
| | GACCACUACCAGCAGAACACCCCCAUCGGCGACGGCCCCGUGCU |
| | GCUGCCCGACAACCACUACCUGAGCACCCAGUCCGCCCUGAGCA |
| | AAGACCCCAACGAGAAGCGCGAUCACAUGGUCCUGCUGGAGUUC |
| | GUGACCGCCGCCGGGAUCACUCUCGGCAUGGACGAGCUGUACAA |
| | GUGAUAAACCGGUGCUGGAGCCUCGGUGGCCAUGCUUCUUGCCC |
| | CUUGGGCCUCCCCCCAGCCCCUCCUCCCCUUCCUGCACCCGUACC |
| | CCCGUGGUCUUUGAAUAAAGUCUGAGUGGGCGGCCUCGAGAAAA |
| | AAAAAAAACAAAAAAAAAAAACCAAAAAAAAAAAAUAAAAAAC |
| | UAAUUAAAACAGCGGAUGGGUACCCCACCAUCCGACCCACUGGG |
| | UGUAGUACUCUGGUACUUCGUACCUUUGUACGCCUGUUCUUCCC |
| | AUUGUACCCUUCCUGAACUUCCAACCCAAGUAACGUUAGAAGCU |
| | CAACAUUUAGUACAACAGGAAGCACCACAUCCAGUGGUGUUUAG |
| | UACAAGCACUUCUGUUUCCCCGGAGCGAGGUAUAGGCUGUACCC |
| | ACUGCCAAAAACCUUUAACCGUUAUCCGCCAACCAACUACGUAA |
| | AAGCUAGUAGUAUUAUGUUUUUAACUAGGCGUUCGAUCAGGUG |
| | GAUUUCCCCUCCACUAGUUUGGUCGAUGAGGCUAGGAAUUCCCC |
| | ACGGGUGACCGUGUCCUAGCCUGCGUGGCGGCCAACCCAGCUUA |
| | UGCUGGGACGCCUUUUUACACAUUUUACAUCUA [symbol] AA |
| | AAGUUAUCAGGCAUGCACCUGGUAGCUAGUCUUUAAACCAAU |
| | AGAUUGCAUCGGUUUAAAAGGCAAGACCGUCAAAUUGCGGGA |
| | AAGGGGUCAACAGCCGUUCAGUACCAAGUCUCAGGGGAAACU |
| | UUGAGAUGGCCUUGCAAAGGGUAUGGUAAUAAGCUGACGGAC |
| | AUGGUCCUAACCACGCAGCCAAGUCCUAAGUCAACAGAUCUU |
| | CUGUUGAUAUGGAUGCAGUUCACAGACUAAAUGUCGGUCGGG |
| | GAAGAUGUAUUCUUCUCAUAAGAUAUAGUCG [symbol] CUCCUUA |
| | AUGGGAGCUAGCGGAUGAAGUGAUGCAACACUGGAGCCGCUG |
| | GACCAGUGGACAAUCGACGGAUAACAGCAUAUCUAGAAAAAAA |
| | AAAAAAAAAAAAAAAAAAAAAAAAAAAAAAAAAAAAAAAAAAA |
| | AAAA |
| SEQ ID NO: 8 | ACCGUCGAUUGUCCACUGGUC [symbol] UUA |
| [R1: Arm I- [symbol] - | UAGACAUGGUGUGAAGACUCGCAUGUGCUUGGUUGUGAUUCCUC |
| Spacer 1- [symbol] - [symbol] | CGGCCCCUGAAUGCGGCUAACCUUAACCCUGGAGCCUUGUGUCA |
| GOI with P10-1 at the 5′ end and | CAAACCAGUGAUGAUAAGGUCGUAAUGAGCAAUUCCGGGACGGG |
| a target site at the 3′ end; Linker | ACCGACUACUUUGGGUGUCCGUGUUUCUUAUUUUUCUUAUUAUU |
| sequence: P1-ex-1-Loop | GUCUUAUGGUCACAGCAUAUAUAUAACAUAUACUGUGAUCAUG |
| sequence (including 5′ portion of | GUGAGCAAGGGCGAGGAGCUGUUCACCGGGGUGGUGCCCAUCCU |
| P10-2)-P1-ex-2 (3′ portion of | GGUCGAGCUGGACGGCGACGUAAACGGCCACAAGUUCAGCGUGU |
| P10-2); [symbol] ; Tetrahymena | CUGGCGAGGGCGAGGGCGAUGCCACCUACGGCAAGCUGACCCUG |
| ribozyme core: SEQ ID NO: 17; | AAGUUCAUCUGCACCACCGGCAAGCUGCCCGUGCCCUGGCCCAC |
| R2: [symbol] -Spacer 2- | CCUCGUGACCACCCUGACCUACGGCGUGCAGUGCUUCAGCCGCU |
| [symbol] -Arm I; Tail] | ACCCCGACCACAUGAAGCAGCACGACUUCUUCAAGUCCGCCAUG |
| related to FIG. 27 | CCCGAAGGCUACGUCCAGGAGCGCACCAUCUUCUUCAAGGACGA |
| | CGGCAACUACAAGACCCGCGCCGAGGUGAAGUUCGAGGGCGACA |
| | CCCUGGUGAACCGCAUCGAGCUGAAGGGCAUCGACUUCAAGGAG |
| | GACGGCAACAUCCUGGGGCACAAGCUGGAGUACAACUACAACAG |
| | CCACAACGUCUAUAUCAUGGCCGACAAGCAGAAGAACGGCAUCA |
| | AGGCGAACUUCAAGAUCCGCCACAACAUCGAGGACGGCAGCGUG |
| | CAGCUCGCCGACCACUACCAGCAGAACACCCCCAUCGGCGACGGC |
| | CCCGUGCUGCUGCCCGACAACCACUACCUGAGCACCCAGUCCGCC |
| | CUGAGCAAAGACCCCAACGAGAAGCGCGAUCACAUGGUCCUGCU |
| | GGAGUUCGUGACCGCCGCCGGGAUCACUCUCGGCAUGGACGAGC |
| | UGUACAAGUGAUAAACCGGUGCUGGAGCCUCGGUGGCCAUGCUU |
| | CUUGCCCCUUGGGCCUCCCCCCAGCCCCUCCUCCCCUUCCUGCAC |
| | CCGUACCCCCGUGGUCUUUGAAUAAAGUCUGAGUGGGCGGCCUC |
| | GAGAAAAAAAAAAAACAAAAAAAAAAAACCAAAAAAAAAAAAU |
| | AAAAAACUAAUUAAAACAGCGGAUGGGUACCCCACCAUCCGACC |
| | CACUGGGUGUAGUACUCUGGUACUUCGUACCUUUGUACGCCUGU |
| | UCUUCCCAUUGUACCCUUCCUGAACUUCCAACCCAAGUAACGUU |
| | AGAAGCUCAACAUUUAGUACAACAGGAAGCACCACAUCCAGUGG |
| | UGUUUAGUACAAGCACUUCUGUUUCCCCGGAGCGAGGUAUAGGC |
| | UGUACCCACUGCCAAAAACCUUUAACCGUUAUCCGCCAACCAAC |
| | UACGUAAAAGCUAGUAGUAUUAUGUUUUUAACUAGGCGUUCGA |
| | UCAGGUGGAUUUCCCCUCCACUAGUUUGGUCGAUGAGGCUAGGA |
| | AUUCCCCACGGGUGACCGUGUCCUAGCCUGCGUGGCGGCCAACC |
| | CAGCUUAUGCUGGGACGCCUUCUUACACAUUUUACAUCUAUAA [symbol] |
| | [symbol] AAAAGUUAUCAGGCAUGCACCUGGUAGCUAGUCUUUAA |
| | ACCAAUAGAUUGCAUCGGUUUAAAAGGCAAGACCGUCAAAUU |
| | GCGGGAAAGGGGUCAACAGCCGUUCAGUACCAAGUCUCAGGG |
| | GAAACUUUGAGAUGGCCUUGCAAAGGGUAUGGUAAUAAGCUG |
| | ACGGACAUGGUCCUAACCACGCAGCCAAGUCCUAAGUCAACA |
| | GAUCUUCUGUUGAUAUGGAUGCAGUUCACAGACUAAAUGUCG |
| | GUCGGGGAAGAUGUAUUCUUCUCAUAAGAUAUAGUCG [symbol] |
| | CUCCUUAAUGGGAGCUAGCGGAUGAAGUGAUGCAACACUGGA |
| | GCCGCUGGGAACUAAUUACCAGUGGACAAUCGACGGAUAACAGC |
| | AUAUCUAGAAAAAAAAAAAAAAAAAAAAAAAAAAAAAAAAAAA |
| | AAAAAAAAAAAAAAAAAAA |
| SEQ ID NO: 9 | UGGAGUAC [symbol] CAUGGCCGACAAGCAGAAGAACGGCAUCAAGGC |
| [R1: Spacer 1- [symbol] - [symbol] | GAACUUCAAGAUCCGCCACAACAUCGAGGACGGCAGCGUGCAGC |
| GOI with P10-1 at the 5′ end and | UCGCCGACCACUACCAGCAGAACACCCCCAUCGGCGACGGCCCCG |
| a target site at the 3′ end; Linker | UGCUGCUGCCCGACAACCACUACCUGAGCACCCAGUCCGCCCUG |
| sequence: P1-ex-1-Loop | AGCAAAGACCCCAACGAGAAGCGCGAUCACAUGGUCCUGCUGGA |
| sequence (including 5′ portion of | GUUCGUGACCGCCGCCGGGAUCACUCUCGGCAUGGACGAGCUGU |
| P10-2)-P1-ex-2 (3′ portion of | ACAAGUGAUAAACCGGUGCUGGAGCCUCGGUGGCCAUGCUUCUU |
| P10-2); [symbol] ; Tetrahymena | GCCCCUUGGGCCUCCCCCCAGCCCCUCCUCCCCUUCCUGCACCCG |
| ribozyme core: SEQ ID NO: 17; | UACCCCCGUGGUCUUUGAAUAAAGUCUGAGUGGGCGGCCUCGAG |
| R2: [symbol] -Spacer 2- | AAAAAAAAAAAACAAAAAAAAAAAACCAAAAAAAAAAAAUAAA |
| Tail] | AAACUAAUUAAAACAGCGGAUGGGUACCCCACCAUCCGACCCAC |
| related to FIG. 30 | UGGGUGUAGUACUCUGGUACUUCGUACCUUUGUACGCCUGUUCU |
| | UCCCAUUGUACCCUUCCUGAACUUCCAACCCAAGUAACGUUAGA |
| | AGCUCAACAUUUAGUACAACAGGAAGCACCACAUCCAGUGGUGU |
| | UUAGUACAAGCACUUCUGUUUCCCCGGAGCGAGGUAUAGGCUGU |
| | ACCCACUGCCAAAAACCUUUAACCGUUAUCCGCCAACCAACUAC |
| | GUAAAAGCUAGUAGUAUUAUGUUUUUAACUAGGCGUUCGAUCA |
| | GGUGGAUUUCCCCUCCACUAGUUUGGUCGAUGAGGCUAGGAAUU |
| | CCCCACGGGUGACCGUGUCCUAGCCUGCGUGGCGGCCAACCCAG |
| | CUUAUGCUGGGACGCCUUUUUAUAGACAUGGUGUGAAGACUCGC |
| | AUGUGCUUGGUUGUGAUUCCUCCGGCCCCUGAAUGCGGCUAACC |
| | UUAACCCUGGAGCCUUGUGUCACAAACCAGUGAUGAUAAGGUCG |
| | UAAUGAGCAAUUCCGGGACGGGACCGACUACUUUGGGUGUCCGU |
| | GUUUCUUAUUUUUCUUAUUAUUGUCUUAUGGUCACAGCAUAUA |
| | UAUAACAUAUACUGUGAUCAUGGUGAGCAAGGGCGAGGAGCUG |
| | UUCACCGGGGUGGUGCCCAUCCUGGUCGAGCUGGACGGCGACGU |
| | AAACGGCCACAAGUUCAGCGUGUCUGGCGAGGGCGAGGGCGAUG |
| | CCACCUACGGCAAGCUGACCCUGAAGUUCAUCUGCACCACCGGC |
| | AAGCUGCCCGUGCCCUGGCCCACCCUCGUGACCACCCUGACCUAC |
| | GGCGUGCAGUGCUUCAGCCGCUACCCCGACCACAUGAAGCAGCA |
| | CGACUUCUUCAAGUCCGCCAUGCCCGAAGGCUACGUCCAGGAGC |
| | GCACCAUCUUCUUCAAGGACGACGGCAACUACAAGACCCGCGCC |
| | GAGGUGAAGUUCGAGGGCGACACCCUGGUGAACCGCAUCGAGCU |
| | GAAGGGCAUCGACUUCAAGGAGGACGGCAACAUCCUGGGGCACA |
| | AGCUGGAGUACAACUACAACAGCCACAACGUCUAUAUCAUCACA |
| | UUUUACAGGCCAUG [symbol] AAAAGUUAUCAGGCAUGCACCUG |
| | GUAGCUAGUCUUUAAACCAAUAGAUUGCAUCGGUUUAAAAGG |
| | CAAGACCGUCAAAUUGCGGGAAAGGGGUCAACAGCCGUUCAG |
| | UACCAAGUCUCAGGGGAAACUUUGAGAUGGCCUUGCAAAGGG |
| | UAUGGUAAUAAGCUGACGGACAUGGUCCUAACCACGCAGCCA |
| | AGUCCUAAGUCAACAGAUCUUCUGUUGAUAUGGAUGCAGUUC |
| | ACAGACUAAAUGUCGGUCGGGGAAGAUGUAUUCUUCUCAUAA |
| | GAUAUAGUCG [symbol] CCUCUCCUUAAUGGGAGCUAGCGGAUGAAG |
| | UGAUGCAACACUGGAGCCGCUGGUAACAGCAUAUCUAGAAAAA |
| | AAAAAAAAAAAAAAAAAAAAAAAAAAAAAAAAAAAAAAAAAAA |
| | AA |
| SEQ ID NO: 10 | ACCGUCGAUUGUCCACUGGUCGAUUAGUUUUCGCAUGGCCGACA |
| [R1: Arm I- [symbol] - | AGCAGAAGAACGGCAUCAAGGCGAACUUCAAGAUCCGCCACAAC |
| [symbol] - [symbol] | AUCGAGGACGGCAGCGUGCAGCUCGCCGACCACUACCAGCAGAA |
| GOI with P10-1 at the 5′ end and | CACCCCCAUCGGCGACGGCCCCGUGCUGCUGCCCGACAACCACUA |
| a target site at the 3′ end; Linker | CCUGAGCACCCAGUCCGCCCUGAGCAAAGACCCCAACGAGAAGC |
| sequence: P1-ex-1-Loop | GCGAUCACAUGGUCCUGCUGGAGUUCGUGACCGCCGCCGGGAUC |
| sequence (including 5′ portion of | ACUCUCGGCAUGGACGAGCUGUACAAGUGAUAAACCGGUGCUGG |
| P10-2)-P1-ex-2 (3′ portion of | AGCCUCGGUGGCCAUGCUUCUUGCCCCUUGGGCCUCCCCCCAGCC |
| P10-2); [symbol] ; Tetrahymena | CCUCCUCCCCUUCCUGCACCCGUACCCCCGUGGUCUUUGAAUAA |
| ribozyme core: SEQ ID NO: 17; | AGUCUGAGUGGGCGGCCUCGAGAAAAAAAAAAAACAAAAAAAA |
| R2: [symbol] - | AAAACCAAAAAAAAAAAAUAAAAAACUAAUUAAAACAGCGGAU |
| [symbol] -Arm I; Tail] | GGGUACCCCACCAUCCGACCCACUGGGUGUAGUACUCUGGUACU |
| related to FIG. 32 | UCGUACCUUUGUACGCCUGUUCUUCCCAUUGUACCCUUCCUGAA |
| | CUUCCAACCCAAGUAACGUUAGAAGCUCAACAUUUAGUACAACA |
| | GGAAGCACCACAUCCAGUGGUGUUUAGUACAAGCACUUCUGUUU |
| | CCCCGGAGCGAGGUAUAGGCUGUACCCACUGCCAAAAACCUUUA |
| | ACCGUUAUCCGCCAACCAACUACGUAAAAGCUAGUAGUAUUAUG |
| | UUUUUAACUAGGCGUUCGAUCAGGUGGAUUUCCCCUCCACUAGU |
| | UUGGUCGAUGAGGCUAGGAAUUCCCCACGGGUGACCGUGUCCUA |
| | GCCUGCGUGGCGGCCAACCCAGCUUAUGCUGGGACGCCUUUUUA |
| | UAGACAUGGUGUGAAGACUCGCAUGUGCUUGGUUGUGAUUCCUC |
| | CGGCCCCUGAAUGCGGCUAACCUUAACCCUGGAGCCUUGUGUCA |
| | CAAACCAGUGAUGAUAAGGUCGUAAUGAGCAAUUCCGGGACGGG |
| | ACCGACUACUUUGGGUGUCCGUGUUUCUUAUUUUUCUUAUUAUU |
| | GUCUUAUGGUCACAGCAUAUAUAUAACAUAUACUGUGAUCAUG |
| | GUGAGCAAGGGCGAGGAGCUGUUCACCGGGGUGGUGCCCAUCCU |
| | GGUCGAGCUGGACGGCGACGUAAACGGCCACAAGUUCAGCGUGU |
| | CUGGCGAGGGCGAGGGCGAUGCCACCUACGGCAAGCUGACCCUG |
| | AAGUUCAUCUGCACCACCGGCAAGCUGCCCGUGCCCUGGCCCAC |
| | CCUCGUGACCACCCUGACCUACGGCGUGCAGUGCUUCAGCCGCU |
| | ACCCCGACCACAUGAAGCAGCACGACUUCUUCAAGUCCGCCAUG |
| | CCCGAAGGCUACGUCCAGGAGCGCACCAUCUUCUUCAAGGACGA |
| | CGGCAACUACAAGACCCGCGCCGAGGUGAAGUUCGAGGGCGACA |
| | CCCUGGUGAACCGCAUCGAGCUGAAGGGCAUCGACUUCAAGGAG |
| | GACGGCAACAUCCUGGGGCACAAGCUGGAGUACAACUACAACAG |
| | CCACAACGUCUAUAUCAUCACAUUUUACAGGCCAUG [symbol] AA |
| | AAGUUAUCAGGCAUGCACCUGGUAGCUAGUCUUUAAACCAAU |
| | AGAUUGCAUCGGUUUAAAAGGCAAGACCGUCAAAUUGCGGGA |
| | AAGGGGUCAACAGCCGUUCAGUACCAAGUCUCAGGGGAAACU |
| | UUGAGAUGGCCUUGCAAAGGGUAUGGUAAUAAGCUGACGGAC |
| | AUGGUCCUAACCACGCAGCCAAGUCCUAAGUCAACAGAUCUU |
| | CUGUUGAUAUGGAUGCAGUUCACAGACUAAAUGUCGGUCGGG |
| | GAAGAUGUAUUCUUCUCAUAAGAUAUAGUCG [symbol] GAACUAAUUA |
| | CCAGUGGACAAUCGACGGAUAACAGCAUAUCUAGAAAAAAAAAA |
| | AUAUCUAGAAAAAAAAAAAAAAAAAAAAAAAAAAAAAAAAAAA |
| | A |
| SEQ ID NO: 11 | [symbol] UUAUAGACAUGGUGUGAAGACUCGCAUGUGCUUGGUUGUGA |
| [R1: [symbol] - [symbol] | UUCCUCCGGCCCCUGAAUGCGGCUAACCUUAACCCUGGAGCCUU |
| GOI with P10-1 at the 5′ end and | GUGUCACAAACCAGUGAUGAUAAGGUCGUAAUGAGCAAUUCCGG |
| a target site at the 3′ end; Linker | GACGGGACCGACUACUUUGGGUGUCCGUGUUUCUUAUUUUUCUU |
| sequence: P1-ex-1-Loop | AUUAUUGUCUUAUGGUCACAGCAUAUAUAUAACAUAUACUGUG |
| sequence (including 5′ portion of | AUCAUGGUGAGCAAGGGCGAGGAGCUGUUCACCGGGGUGGUGCC |
| P10-2)-P1-ex-2 (3′ portion of | CAUCCUGGUCGAGCUGGACGGCGACGUAAACGGCCACAAGUUCA |
| P10-2); [symbol] ; Pneumocystis | GCGUGUCUGGCGAGGGCGAGGGCGAUGCCACCUACGGCAAGCUG |
| carinii ribozyme core: SEQ ID | ACCCUGAAGUUCAUCUGCACCACCGGCAAGCUGCCCGUGCCCUG |
| NO: 19; | GCCCACCCUCGUGACCACCCUGACCUACGGCGUGCAGUGCUUCA |
| R2: [symbol] ; Tail] | GCCGCUACCCCGACCACAUGAAGCAGCACGACUUCUUCAAGUCC |
| related to FIG. 35 | GCCAUGCCCGAAGGCUACGUCCAGGAGCGCACCAUCUUCUUCAA |
| | GGACGACGGCAACUACAAGACCCGCGCCGAGGUGAAGUUCGAGG |
| | GCGACACCCUGGUGAACCGCAUCGAGCUGAAGGGCAUCGACUUC |
| | AAGGAGGACGGCAACAUCCUGGGGCACAAGCUGGAGUACAACUA |
| | CAACAGCCACAACGUCUAUAUCAUGGCCGACAAGCAGAAGAACG |
| | GCAUCAAGGCGAACUUCAAGAUCCGCCACAACAUCGAGGACGGC |
| | AGCGUGCAGCUCGCCGACCACUACCAGCAGAACACCCCCAUCGG |
| | CGACGGCCCCGUGCUGCUGCCCGACAACCACUACCUGAGCACCCA |
| | GUCCGCCCUGAGCAAAGACCCCAACGAGAAGCGCGAUCACAUGG |
| | UCCUGCUGGAGUUCGUGACCGCCGCCGGGAUCACUCUCGGCAUG |
| | GACGAGCUGUACAAGUGAUAAACCGGUGCUGGAGCCUCGGUGGC |
| | CAUGCUUCUUGCCCCUUGGGCCUCCCCCCAGCCCCUCCUCCCCUU |
| | CCUGCACCCGUACCCCCGUGGUCUUUGAAUAAAGUCUGAGUGGG |
| | CGGCCUCGAGAAAAAAAAAAAACAAAAAAAAAAAACCAAAAAA |
| | AAAAAAUAAAAAACUAAUUAAAACAGCGGAUGGGUACCCCACCA |
| | UCCGACCCACUGGGUGUAGUACUCUGGUACUUCGUACCUUUGUA |
| | CGCCUGUUCUUCCCAUUGUACCCUUCCUGAACUUCCAACCCAAG |
| | UAACGUUAGAAGCUCAACAUUUAGUACAACAGGAAGCACCACAU |
| | CCAGUGGUGUUUAGUACAAGCACUUCUGUUUCCCCGGAGCGAGG |
| | UAUAGGCUGUACCCACUGCCAAAAACCUUUAACCGUUAUCCGCC |
| | AACCAACUACGUAAAAGCUAGUAGUAUUAUGUUUUUAACUAGG |
| | CGUUCGAUCAGGUGGAUUUCCCCUCCACUAGUUUGGUCGAUGAG |
| | GCUAGGAAUUCCCCACGGGUGACCGUGUCCUAGCCUGCGUGGCG |
| | GCCAACCCAGCUUAUGCUGGGACGCCUUUUACCUUCUAUAA [symbol] |
| | [symbol] GAAAGCGGCGUGAAAACGUUAGCUAGUGAUCUGGAAUAA |
| | AUUCAGAUUGCGACACUGUCAAAUUGCGGGGAAGCCCUAAAU |
| | AUUCAACUACUAAGCAGUUUGUGGAAACACAGCUGUGGCCGA |
| | GUUAAUAGCCCUGGGUAUAGUAACAAUGUUGAAUAUGAAUCU |
| | UUUGGGAGAUGAAAUGGGUGAUCCGCAGCCAAGUCCUAAGGG |
| | CAUUUUUGUCUAUGGAUGCAGUUCAACGACUAGAUGGCAGUG |
| | GGUAUUGUAAGGAAUUGCAGUUUUCUUGCAGUGCUUAAGGUA |
| | UAGUCU [symbol] UAACAGCAUAUCUAGAAAAAAAAAAAAAAAAAAAA |
| | AAAAAAAAAAAAAAAAAAAAAAAAAAAAAAAAAA |
| SEQ ID NO: 12 (GenBank | AAAUAGCAAUAUUUACCUUUGGAGGGAAAAGUUAUCAGGCAUGC |
| accession number: V01416.1, | ACCUGGUAGCUAGUCUUUAAACCAAUAGAUUGCAUCGGUUUAAA |
| fragment; Tetrahymena | AGGCAAGACCGUCAAAUUGCGGGAAAGGGGUCAACAGCCGUUCA |
| thermophila group I intron) | GUACCAAGUCUCAGGGGAAACUUUGAGAUGGCCUUGCAAAGGGU |
| IGS-5′ half of P9.0-Spacer 2- | AUGGUAAUAAGCUGACGGACAUGGUCCUAACCACGCAGCCAAGU |
| 5′ half of P9.2-Spacer 3-3′ | CCUAAGUCAACAGAUCUUCUGUUGAUAUGGAUGCAGUUCACAGA |
| half of P9.2-Spacer 1-3′ half | CUAAAUGUCGGUCGGGGAAGAUGUAUUCUUCUCAUAAGAUAUA |
| of P9.0-ωG | GUCGGACCUCUCCUUAAUGGGAGCUAGCGGAUGAAGUGAUGCAA |
| | CACUGGAGCCGCUGGGAACUAAUUUGUAUGCGAAAGUAUAUUG |
| | AUUAGUUUUGGAGUACUCG |
| SEQ ID NO: 13 (5′ homology | ACCGUCGAUUGUCCACUGGUC |
| arm sequence, Arm I) | |
| SEQ ID NO: 14 (3′ homology | ACCAGUGGACAAUCGACGGA |
| arm sequence, Arm I) | |
| SEQ ID NO: 15 (5′ homology | GGACGGGCAAG |
| arm sequence, Arm II) | |
| SEQ ID NO: 16 (3′ homology | CUUGCCCGUCC |
| arm sequence, Arm II) | |
| SEQ ID NO: 17 (ribozyme core | AAAAGUUAUCAGGCAUGCACCUGGUAGCUAGUCUUUAAACCAAU |
| derived from a Tetrahymena | AGAUUGCAUCGGUUUAAAAGGCAAGACCGUCAAAUUGCGGGAA |
| thermophila group I intron, from | AGGGGUCAACAGCCGUUCAGUACCAAGUCUCAGGGGAAACUUUG |
| IGS end to the sequence before the | AGAUGGCCUUGCAAAGGGUAUGGUAAUAAGCUGACGGACAUGG |
| P9.0 duplex) | UCCUAACCACGCAGCCAAGUCCUAAGUCAACAGAUCUUCUGUUG |
| | AUAUGGAUGCAGUUCACAGACUAAAUGUCGGUCGGGGAAGAUG |
| | UAUUCUUCUCAUAAGAUAUAGUCG |
| SEQ ID NO: 18 (GenBank | AAAAGUUAUCAGGCAUGCACCUGGUAGCUAGUCUUUAAACCAAU |
| accession number: V01416.1, | AGAUUGCAUCGGUUUAAAAGGCAAGACCGUCAAAUUGCGGGAA |
| fragment; Tetrahymena | AGGGGUCAACAGCCGUUCAGUACCAAGUCUCAGGGGAAACUUUG |
| thermophila group I intron | AGAUGGCCUUGCAAAGGGUAUGGUAAUAAGCUGACGGACAUGG |
| fragment, from IGS end to ωG) | UCCUAACCACGCAGCCAAGUCCUAAGUCAACAGAUCUUCUGUUG |
| | AUAUGGAUGCAGUUCACAGACUAAAUGUCGGUCGGGGAAGAUG |
| | UAUUCUUCUCAUAAGAUAUAGUCGGACCUCUCCUUAAUGGGAGC |
| | UAGCGGAUGAAGUGAUGCAACACUGGAGCCGCUGGGAACUAAUU |
| | UGUAUGCGAAAGUAUAUUGAUUAGUUUUGGAGUACUCG |
| SEQ ID NO: 19 (ribozyme core | GAAAGCGGCGUGAAAACGUUAGCUAGUGAUCUGGAAUAAAUUC |
| derived from a Pneumocystis | AGAUUGCGACACUGUCAAAUUGCGGGGAAGCCCUAAAUAUUCAA |
| carinii group I intron, from IGS | CUACUAAGCAGUUUGUGGAAACACAGCUGUGGCCGAGUUAAUAG |
| end to the sequence before the | CCCUGGGUAUAGUAACAAUGUUGAAUAUGAAUCUUUUGGGAGA |
| P9.0 duplex) | UGAAAUGGGUGAUCCGCAGCCAAGUCCUAAGGGCAUUUUUGUCU |
| | AUGGAUGCAGUUCAACGACUAGAUGGCAGUGGGUAUUGUAAGG |
| | AAUUGCAGUUUUCUUGCAGUGCUUAAGGUAUAGUCU |
| SEQ ID NO: 20 | GACCUCUCCUUAAUGGGAGCUAGCGGAUGAAGUGAUGCAACACU |
| (R2 of SEQ ID NOs: 1, 2 and 8) | GGAGCCGCUGGGAACUAAUUACCAGUGGACAAUCGACGGA |
| SEQ ID NO: 21 | ACCGUCGAUUGUCCACUGGUCGAUUAGUUUUGGAGUACUCG |
| (R1 of SEQ ID NOs: 1, 2 and 8) | |
| SEQ ID NO: 22 | GACCUCUCCUUAAUGGGAGCUAGCGGAUGAAGUGAUGCAACACU |
| (R2 of SEQ ID NO: 7) | GGAGCCGCUGGACCAGUGGACAAUCGACGGA |
| SEQ ID NO: 23 | ACCGUCGAUUGUCCACUGGUCUGGAGUACUCG |
| (R1 of SEQ ID NO: 7) | |
| SEQ ID NO: 24 | GACCUCUCCUUAAUGGGAGCUAGCGGAUGAAGUGAUGCAACACU |
| (R2 of SEQ ID NO: 9) | GGAGCCGCUGG |
| SEQ ID NO: 25 | UGGAGUACUCG |
| (R1 of SEQ ID NO: 9) | |
| SEQ ID NO: 26 | GAGAACUAAUUACCAGUGGACAAUCGACGGA |
| (R2 of SEQ ID NO: 10) | |
| SEQ ID NO: 27 | ACCGUCGAUUGUCCACUGGUCGAUUAGUUUUCG |
| (R1 of SEQ ID NO: 10) | |
| SEQ ID NO: 28 | GUCAAGGCACCAGUGGACAAUCGACGGA |
| (R2 of SEQ ID NO: 3) | |
| SEQ ID NO: 29 | ACCGUCGAUUGUCCACUGGUCGCCUUGACG |
| (R1 of SEQ ID NO: 3) | |
| SEQ ID NO: 30 | AUCAAGGCACCAGUGGACAAUCGACGGA |
| (R2 of SEQ ID NO: 6) | |
| SEQ ID NO: 31 | ACCGUCGAUUGUCCACUGGUCGCCUUGGUG |
| (R1 of SEQ ID NO: 6) | |
| SEQ ID NO: 32 (GenBank: | CACCUUCUGAGGGUCAUGAAAGCGGCGUGAAAACGUUAGCUAGU |
| AF236872.1, fragment; | GAUCUGGAAUAAAUUCAGAUUGCGACACUGUCAAAUUGCGGGG |
| Pneumocystis carinii group I | AAGCCCUAAAGAUUCAACUACUAAGCAGUUUGUGGAAACACAGC |
| intron, IGS, 5′ half of P9.0 and [symbol] | UGUGGCCGAGUUAAUAGCCCUGGGUAUAGUAACAAUGUUGAAU |
| [symbol] are marked) | AUGAAUCUUUUGCGAGAUGAAAUGGGUGAUCCGCAGCCAAGUCC |
| | UAAGGGCAUUUUUGUCUAUGGAUGCAGUUCAACGACUAGAUGG |
| | CAGUGGGUAUUGUAAGGAAUUGCAGUUUUCUUGCAGUGCUUAA |
| | GGUAUAGUCUAUCCUCUUUCGAAAGAAAGAGUAUAUU [symbol] |
| SEQ ID NO: 33 (GenBank: | CAACCUUUUGAGGGUCAUGAAAGCAGCGUGAAAACGUUUGCUAG |
| L13615.1, fragment; | UGGUCAAGUUGGGUAUUCUGAUUUGACUGCGACACUGUCAAAU |
| Pneumocystis carinii group I | UGCGGGGAAGCCCUAAAGCCUUAUUCACCAAGCAAUUGUGGAAA |
| intron) | CACUCUUGUGGCCAGGUUAAUAGCCUCGGGUAUGGUAACAGUAG |
| | UAAGGAUAAAUGUGAAAAAUGGGUUAUCCGCAGCCAAAUCCUA |
| | AGGGGAAAAAGAAUUACAAAUCCGUAUUUUCAUCCUAUGGAUG |
| | CAGUUCAACGACUAGACGGCAGUGGGUACUGCUCUUUUUUACUC |
| | UGAGUAGUGCUUAAGGUAUAGUCUGUCCUUUUCUGAAAAGAGA |
| | GGGAGGGGAGGUG |
| SEQ ID NO: 34 (GenBank: | CAACCUUUUGAGGGUCAUUAAAGCGGCGUGAAAACGUUCGCUAG |
| L13614.1, fragment; | UGAUCUGAAGCCUUUUCAGGUUGCGACACUGUCAAAUUGCGGGG |
| Pneumocystis wakefieldiae group I | AAGCCCUAAAGAUUCAACUACUAAGCAGCUUGUGGAAACACAGC |
| intron) | UGUGGCCGAGUUAAUAGCCCUGGGUAUAGUAACAAUGUUGAAU |
| | AUGAAUCUUGAUUGAAGAUGAAAUGGGUGAUCCGCAGCCAAGU |
| | CCUAAGGACGUAUAAUGUCUAUGGAUGCAGUUCAACGACUAGAU |
| | GGCAGUGGGUGUUGUUAAGACUUAGGUUUUUACAAUGCUUAAG |
| | GUAUAGUCUAUUCUCUAUCGAAAGAUAGCGUAUGGUG |
| SEQ ID NO: 35 (GenBank: | UUUUUAUUGGUUCUUCUGCAGUGCGCCAAAGGAAGCCUUAGCAG |
| X13687.1, fragment; | CCUGAAAGGGUGUAUCUCCGCGACUAUAAAUAAAAAGGGGAUU |
| Pneumocystis carinii group I | UUAAAUGCUAGUCUGAUAAAAAAAGGCGACAUUGCCAAAUUGC |
| intron) | GGGAAGUCCCUAAAGAUUCAACUACUAAGCAGCUUGUGGAAACA |
| | CAGUUGUGGCCGAGUUAAUAGCCCUGGGUAUAGUAACAAUGUU |
| | GAAUAUGACUCUUAAUUGAGGAAAUGGGUGAUCCGCAGCCAAA |
| | UCCUAAGGACAUUUUAUUGUCUAUGGAUGCAGUUCACAGGCCAG |
| | AUGGCAAUGGGUAUCCUAGUGGGAUAUAUAUAUAUGGAUGCUU |
| | AAGAUAUGGUCGAGCUUCUCUCGAAAGAGAGGAGGUAGCACUG |
| SEQ ID NO: 36 (GenBank: | CACCUUUUGAGGGUCAUGAAAGCGGCGCGAAAGUGUUAGCUAGU |
| M86760, fragment; Pneumocystis | GAUCCGAAAAAUAAAUUCGGGUUGCGACACUGUCAAAUUGCGGG |
| carinii group I intron) | GAGUCCCUAAAGAUUCAACUACUAAGCAGCUUGUGGAAACACAG |
| | UUGUGGCCGAGUUAAUAGCCCUGGGUAUAGUAACAAUGUUGAA |
| | UAUGACUCUUAAUUGAGGAAAUGGGUGAUCCGCAGCCAAAUCCU |
| | AAGGACAUUUUAUUGUCUAUGGAUGCAGUUCAGCGACUAGACG |
| | GCAGUGGGUAUUGUAGAGAUAUGGGGUUAUUUAUGGCCUUAUC |
| | UACAAUGCUUAAGGUAUAGUCUAAUCUCUUUCGAAAGAAAGAG |
| | UAGUGUG |
| SEQ ID NO: 37 (5′ homology arm | ACCGUCGAUUGUCCACUGGUCGCCUUG |
| sequence, Arm III) | |
| SEQ ID NO: 38 (3′ homology arm | CAAGGCACCAGUGGACAAUCGACGGA |
| sequence, Arm III) | |
| SEQ ID NO: 39 | [symbol] CAUGGCCGACAAGCAGAAGAACGGCAUCAAGGCGAACUUCA |
| [R1: [symbol] - [symbol] | AGAUCCGCCACAACAUCGAGGACGGCAGCGUGCAGCUCGCCGAC |
| GOI with P10-1 at the 5′ end and | CACUACCAGCAGAACACCCCCAUCGGCGACGGCCCCGUGCUGCU |
| a target site at the 3′ end; Linker | GCCCGACAACCACUACCUGAGCACCCAGUCCGCCCUGAGCAAAG |
| sequence: P1-ex-1-Loop | ACCCCAACGAGAAGCGCGAUCACAUGGUCCUGCUGGAGUUCGUG |
| sequence (including 5′ portion of | ACCGCCGCCGGGAUCACUCUCGGCAUGGACGAGCUGUACAAGUG |
| P10-2)-P1-ex-2 (3′ portion of | AUAAACCGGUGCUGGAGCCUCGGUGGCCAUGCUUCUUGCCCCUU |
| P10-2); [symbol] ; Tetrahymena | GGGCCUCCCCCCAGCCCCUCCUCCCCUUCCUGCACCCGUACCCCC |
| ribozyme core: SEQ ID NO: 17; | GUGGUCUUUGAAUAAAGUCUGAGUGGGCGGCCUCGAGAAAAAA |
| R2: [symbol] ; Tail] | AAAAAACAAAAAAAAAAAACCAAAAAAAAAAAAUAAAAAACUA |
| related to FIG. 37 | AUUAAAACAGCGGAUGGGUACCCCACCAUCCGACCCACUGGGUG |
| | UAGUACUCUGGUACUUCGUACCUUUGUACGCCUGUUCUUCCCAU |
| | UGUACCCUUCCUGAACUUCCAACCCAAGUAACGUUAGAAGCUCA |
| | ACAUUUAGUACAACAGGAAGCACCACAUCCAGUGGUGUUUAGUA |
| | CAAGCACUUCUGUUUCCCCGGAGCGAGGUAUAGGCUGUACCCAC |
| | UGCCAAAAACCUUUAACCGUUAUCCGCCAACCAACUACGUAAAA |
| | GCUAGUAGUAUUAUGUUUUUAACUAGGCGUUCGAUCAGGUGGA |
| | UUUCCCCUCCACUAGUUUGGUCGAUGAGGCUAGGAAUUCCCCAC |
| | GGGUGACCGUGUCCUAGCCUGCGUGGCGGCCAACCCAGCUUAUG |
| | CUGGGACGCCUUUUUAUAGACAUGGUGUGAAGACUCGCAUGUGC |
| | UUGGUUGUGAUUCCUCCGGCCCCUGAAUGCGGCUAACCUUAACC |
| | CUGGAGCCUUGUGUCACAAACCAGUGAUGAUAAGGUCGUAAUGA |
| | GCAAUUCCGGGACGGGACCGACUACUUUGGGUGUCCGUGUUUCU |
| | UAUUUUUCUUAUUAUUGUCUUAUGGUCACAGCAUAUAUAUAAC |
| | AUAUACUGUGAUCAUGGUGAGCAAGGGCGAGGAGCUGUUCACCG |
| | GGGUGGUGCCCAUCCUGGUCGAGCUGGACGGCGACGUAAACGGC |
| | CACAAGUUCAGCGUGUCUGGCGAGGGCGAGGGCGAUGCCACCUA |
| | CGGCAAGCUGACCCUGAAGUUCAUCUGCACCACCGGCAAGCUGC |
| | CCGUGCCCUGGCCCACCCUCGUGACCACCCUGACCUACGGCGUGC |
| | AGUGCUUCAGCCGCUACCCCGACCACAUGAAGCAGCACGACUUC |
| | UUCAAGUCCGCCAUGCCCGAAGGCUACGUCCAGGAGCGCACCAU |
| | CUUCUUCAAGGACGACGGCAACUACAAGACCCGCGCCGAGGUGA |
| | AGUUCGAGGGCGACACCCUGGUGAACCGCAUCGAGCUGAAGGGC |
| | AUCGACUUCAAGGAGGACGGCAACAUCCUGGGGCACAAGCUGGA |
| | GUACAACUACAACAGCCACAACGUCUAUAUCAUCACAUUUUACA |
| | GGCCAUG [symbol] AAAAGUUAUCAGGCAUGCACCUGGUAGCUA |
| | GUCUUUAAACCAAUAGAUUGCAUCGGUUUAAAAGGCAAGACC |
| | GUCAAAUUGCGGGAAAGGGGUCAACAGCCGUUCAGUACCAAG |
| | UCUCAGGGGAAACUUUGAGAUGGCCUUGCAAAGGGUAUGGUA |
| | AUAAGCUGACGGACAUGGUCCUAACCACGCAGCCAAGUCCUA |
| | AGUCAACAGAUCUUCUGUUGAUAUGGAUGCAGUUCACAGACU |
| | AAAUGUCGGUCGGGGAAGAUGUAUUCUUCUCAUAAGAUAUAG |
| | UCG [symbol] UAACAGCAUAUCUAGAAAAAAAAAAAAAAAAAAAAAAA |
| | AAAAAAAAAAAAAAAAAAAAAAAAAAAAAAA |
| SEQ ID NO: 40 | AACACCAA [symbol] CAUGGCCGACAAGCAGAAGAACGGCAUCAAGGCGA |
| [R1: Loop 1- [symbol] | ACUUCAAGAUCCGCCACAACAUCGAGGACGGCAGCGUGCAGCUC |
| GOI with P10-1 at the 5′ end and | GCCGACCACUACCAGCAGAACACCCCCAUCGGCGACGGCCCCGUG |
| a target site at the 3′ end; Linker | CUGCUGCCCGACAACCACUACCUGAGCACCCAGUCCGCCCUGAGC |
| sequence: P1-ex-1-Loop | AAAGACCCCAACGAGAAGCGCGAUCACAUGGUCCUGCUGGAGUU |
| sequence (including 5′ portion of | CGUGACCGCCGCCGGGAUCACUCUCGGCAUGGACGAGCUGUACA |
| P10-2)-P1-ex-2 (3′ portion of | AGUGAUAAACCGGUGCUGGAGCCUCGGUGGCCAUGCUUCUUGCC |
| P10-2); [symbol] ; Tetrahymena | CCUUGGGCCUCCCCCCAGCCCCUCCUCCCCUUCCUGCACCCGUAC |
| ribozyme core: SEQ ID NO: 17; | CCCCGUGGUCUUUGAAUAAAGUCUGAGUGGGCGGCCUCGAGAAA |
| R2: Loop 2; Tail] | AAAAAAAAACAAAAAAAAAAAACCAAAAAAAAAAAAUAAAAAA |
| related to FIG. 40 | CUAAUUAAAACAGCGGAUGGGUACCCCACCAUCCGACCCACUGG |
| | GUGUAGUACUCUGGUACUUCGUACCUUUGUACGCCUGUUCUUCC |
| | CAUUGUACCCUUCCUGAACUUCCAACCCAAGUAACGUUAGAAGC |
| | UCAACAUUUAGUACAACAGGAAGCACCACAUCCAGUGGUGUUUA |
| | GUACAAGCACUUCUGUUUCCCCGGAGCGAGGUAUAGGCUGUACC |
| | CACUGCCAAAAACCUUUAACCGUUAUCCGCCAACCAACUACGUA |
| | AAAGCUAGUAGUAUUAUGUUUUUAACUAGGCGUUCGAUCAGGU |
| | GGAUUUCCCCUCCACUAGUUUGGUCGAUGAGGCUAGGAAUUCCC |
| | CACGGGUGACCGUGUCCUAGCCUGCGUGGCGGCCAACCCAGCUU |
| | AUGCUGGGACGCCUUUUUAUAGACAUGGUGUGAAGACUCGCAUG |
| | UGCUUGGUUGUGAUUCCUCCGGCCCCUGAAUGCGGCUAACCUUA |
| | ACCCUGGAGCCUUGUGUCACAAACCAGUGAUGAUAAGGUCGUAA |
| | UGAGCAAUUCCGGGACGGGACCGACUACUUUGGGUGUCCGUGUU |
| | UCUUAUUUUUCUUAUUAUUGUCUUAUGGUCACAGCAUAUAUAU |
| | AACAUAUACUGUGAUCAUGGUGAGCAAGGGCGAGGAGCUGUUC |
| | ACCGGGGUGGUGCCCAUCCUGGUCGAGCUGGACGGCGACGUAAA |
| | CGGCCACAAGUUCAGCGUGUCUGGCGAGGGCGAGGGCGAUGCCA |
| | CCUACGGCAAGCUGACCCUGAAGUUCAUCUGCACCACCGGCAAG |
| | CUGCCCGUGCCCUGGCCCACCCUCGUGACCACCCUGACCUACGGC |
| | GUGCAGUGCUUCAGCCGCUACCCCGACCACAUGAAGCAGCACGA |
| | CUUCUUCAAGUCCGCCAUGCCCGAAGGCUACGUCCAGGAGCGCA |
| | CCAUCUUCUUCAAGGACGACGGCAACUACAAGACCCGCGCCGAG |
| | GUGAAGUUCGAGGGCGACACCCUGGUGAACCGCAUCGAGCUGAA |
| | GGGCAUCGACUUCAAGGAGGACGGCAACAUCCUGGGGCACAAGC |
| | UGGAGUACAACUACAACAGCCACAACGUCUAUAUCAUCACAUUU |
| | UACAGGCCAUG [symbol] AAAAGUUAUCAGGCAUGCACCUGGUA |
| | GCUAGUCUUUAAACCAAUAGAUUGCAUCGGUUUAAAAGGCAA |
| | GACCGUCAAAUUGCGGGAAAGGGGUCAACAGCCGUUCAGUAC |
| | CAAGUCUCAGGGGAAACUUUGAGAUGGCCUUGCAAAGGGUAU |
| | GGUAAUAAGCUGACGGACAUGGUCCUAACCACGCAGCCAAGU |
| | CCUAAGUCAACAGAUCUUCUGUUGAUAUGGAUGCAGUUCACA |
| | GACUAAAUGUCGGUCGGGGAAGAUGUAUUCUUCUCAUAAGAU |
| | AUAGUCGAACCACAAUAACAGCAUAUCUAGAAAAAAAAAAAAA |
| | AAAAAAAAAAAAAAAAAAAAAAAAAAAAAAAAAAAAAAAAA |
| SEQ ID NO: 41 | ACCGUCGAUUGUCCACUGGUCGAUUAGUUUAACACCAAGCAUGG |
| [R1: Arm I- [symbol] - | CCGACAAGCAGAAGAACGGCAUCAAGGCGAACUUCAAGAUCCGC |
| Loop 1- [symbol] | CACAACAUCGAGGACGGCAGCGUGCAGCUCGCCGACCACUACCA |
| GOI with P10-1 at the 5′ end and | GCAGAACACCCCCAUCGGCGACGGCCCCGUGCUGCUGCCCGACA |
| a target site at the 3′ end; Linker | ACCACUACCUGAGCACCCAGUCCGCCCUGAGCAAAGACCCCAAC |
| sequence: P1-ex-1-Loop | GAGAAGCGCGAUCACAUGGUCCUGCUGGAGUUCGUGACCGCCGC |
| sequence (including 5′ portion of | CGGGAUCACUCUCGGCAUGGACGAGCUGUACAAGUGAUAAACCG |
| P10-2)-P1-ex-2 (3′ portion of | GUGCUGGAGCCUCGGUGGCCAUGCUUCUUGCCCCUUGGGCCUCC |
| P10-2); [symbol] ; Tetrahymena | CCCCAGCCCCUCCUCCCCUUCCUGCACCCGUACCCCCGUGGUCUU |
| ribozyme core: SEQ ID NO: 17; | UGAAUAAAGUCUGAGUGGGCGGCCUCGAGAAAAAAAAAAAACA |
| R2: Loop 2-5′ half of P9.2- | AAAAAAAAAAACCAAAAAAAAAAAAUAAAAAACUAAUUAAAAC |
| Arm I; Tail] | AGCGGAUGGGUACCCCACCAUCCGACCCACUGGGUGUAGUACUC |
| related to FIG. 43 | UGGUACUUCGUACCUUUGUACGCCUGUUCUUCCCAUUGUACCCU |
| | UCCUGAACUUCCAACCCAAGUAACGUUAGAAGCUCAACAUUUAG |
| | UACAACAGGAAGCACCACAUCCAGUGGUGUUUAGUACAAGCACU |
| | UCUGUUUCCCCGGAGCGAGGUAUAGGCUGUACCCACUGCCAAAA |
| | ACCUUUAACCGUUAUCCGCCAACCAACUACGUAAAAGCUAGUAG |
| | UAUUAUGUUUUUAACUAGGCGUUCGAUCAGGUGGAUUUCCCCUC |
| | CACUAGUUUGGUCGAUGAGGCUAGGAAUUCCCCACGGGUGACCG |
| | UGUCCUAGCCUGCGUGGCGGCCAACCCAGCUUAUGCUGGGACGC |
| | CUUUUUAUAGACAUGGUGUGAAGACUCGCAUGUGCUUGGUUGU |
| | GAUUCCUCCGGCCCCUGAAUGCGGCUAACCUUAACCCUGGAGCC |
| | UUGUGUCACAAACCAGUGAUGAUAAGGUCGUAAUGAGCAAUUCC |
| | GGGACGGGACCGACUACUUUGGGUGUCCGUGUUUCUUAUUUUUC |
| | UUAUUAUUGUCUUAUGGUCACAGCAUAUAUAUAACAUAUACUG |
| | UGAUCAUGGUGAGCAAGGGCGAGGAGCUGUUCACCGGGGUGGU |
| | GCCCAUCCUGGUCGAGCUGGACGGCGACGUAAACGGCCACAAGU |
| | UCAGCGUGUCUGGCGAGGGCGAGGGCGAUGCCACCUACGGCAAG |
| | CUGACCCUGAAGUUCAUCUGCACCACCGGCAAGCUGCCCGUGCC |
| | CUGGCCCACCCUCGUGACCACCCUGACCUACGGCGUGCAGUGCU |
| | UCAGCCGCUACCCCGACCACAUGAAGCAGCACGACUUCUUCAAG |
| | UCCGCCAUGCCCGAAGGCUACGUCCAGGAGCGCACCAUCUUCUU |
| | CAAGGACGACGGCAACUACAAGACCCGCGCCGAGGUGAAGUUCG |
| | AGGGCGACACCCUGGUGAACCGCAUCGAGCUGAAGGGCAUCGAC |
| | UUCAAGGAGGACGGCAACAUCCUGGGGCACAAGCUGGAGUACAA |
| | CUACAACAGCCACAACGUCUAUAUCAUCACAUUUUACAGGCCAU |
| | G [symbol] AAAAGUUAUCAGGCAUGCACCUGGUAGCUAGUCUU |
| | UAAACCAAUAGAUUGCAUCGGUUUAAAAGGCAAGACCGUCAA |
| | AUUGCGGGAAAGGGGUCAACAGCCGUUCAGUACCAAGUCUCA |
| | GGGGAAACUUUGAGAUGGCCUUGCAAAGGGUAUGGUAAUAAG |
| | CUGACGGACAUGGUCCUAACCACGCAGCCAAGUCCUAAGUCA |
| | ACAGAUCUUCUGUUGAUAUGGAUGCAGUUCACAGACUAAAUG |
| | UCGGUCGGGGAAGAUGUAUUCUUCUCAUAAGAUAUAGUCGAA |
| | CCACAAGAACUAAUUACCAGUGGACAAUCGACGGAUAACAGCAU |
| | AUCUAGAAAAAAAAAAAAAAAAAAAAAAAAAAAAAAAAAAAAA |
| | AAAAAAAAAAAAAAAAA |
| SEQ ID NO: 42 (R1 of SEQ ID | ACCGUCGAUUGUCCACUGGUCGAUUAGUUUAACACCAAG |
| NO: 41) | |
| SEQ ID NO: 43 (R2 of SEQ ID | AACCACAAGAACUAAUUACCAGUGGACAAUCGACGGA |
| NO: 41) | |
| SEQ ID NO: 44 | ACCGUCGAUUGUCCACUGGUCGAUUAGUUUUGGAGUAC [symbol] CAU |
| [R1: Arm I- [symbol] - | GGCCGACAAGCAGAAGAACGGCAUCAAGGCGAACUUCAAGAUCC |
| Spacer 1- [symbol] - [symbol] | GCCACAACAUCGAGGACGGCAGCGUGCAGCUCGCCGACCACUAC |
| GOI with P10-1 at the 5′ end and | CAGCAGAACACCCCCAUCGGCGACGGCCCCGUGCUGCUGCCCGAC |
| a target site at the 3′ end; Linker | AACCACUACCUGAGCACCCAGUCCGCCCUGAGCAAAGACCCCAA |
| sequence: P1-ex-1-Loop | CGAGAAGCGCGAUCACAUGGUCCUGCUGGAGUUCGUGACCGCCG |
| sequence (including 5′ portion of | CCGGGAUCACUCUCGGCAUGGACGAGCUGUACAAGUGAUAAACC |
| P10-2)-P1-ex-2 (3′ portion of | GGUGCUGGAGCCUCGGUGGCCAUGCUUCUUGCCCCUUGGGCCUC |
| P10-2); [symbol] ; Tetrahymena | CCCCCAGCCCCUCCUCCCCUUCCUGCACCCGUACCCCCGUGGUCU |
| ribozyme core: SEQ ID NO: 17; | UUGAAUAAAGUCUGAGUGGGCGGCCUCGAGAAAAAAAAAAAAC |
| R2: [symbol] -Spacer 2- | AAAAAAAAAAAACCAAAAAAAAAAAAUAAAAAACUAAUUAAAA |
| 5′ half of P9.2-Arm I; Tail] | CAGCGGAUGGGUACCCCACCAUCCGACCCACUGGGUGUAGUACU |
| related to FIG. 46 | CUGGUACUUCGUACCUUUGUACGCCUGUUCUUCCCAUUGUACCC |
| | UUCCUGAACUUCCAACCCAAGUAACGUUAGAAGCUCAACAUUUA |
| | GUACAACAGGAAGCACCACAUCCAGUGGUGUUUAGUACAAGCAC |
| | UUCUGUUUCCCCGGAGCGAGGUAUAGGCUGUACCCACUGCCAAA |
| | AACCUUUAACCGUUAUCCGCCAACCAACUACGUAAAAGCUAGUA |
| | GUAUUAUGUUUUUAACUAGGCGUUCGAUCAGGUGGAUUUCCCCU |
| | CCACUAGUUUGGUCGAUGAGGCUAGGAAUUCCCCACGGGUGACC |
| | GUGUCCUAGCCUGCGUGGCGGCCAACCCAGCUUAUGCUGGGACG |
| | CCUUUUUAUAGACAUGGUGUGAAGACUCGCAUGUGCUUGGUUG |
| | UGAUUCCUCCGGCCCCUGAAUGCGGCUAACCUUAACCCUGGAGC |
| | CUUGUGUCACAAACCAGUGAUGAUAAGGUCGUAAUGAGCAAUUC |
| | CGGGACGGGACCGACUACUUUGGGUGUCCGUGUUUCUUAUUUUU |
| | CUUAUUAUUGUCUUAUGGUCACAGCAUAUAUAUAACAUAUACU |
| | GUGAUCAUGGUGAGCAAGGGCGAGGAGCUGUUCACCGGGGUGG |
| | UGCCCAUCCUGGUCGAGCUGGACGGCGACGUAAACGGCCACAAG |
| | UUCAGCGUGUCUGGCGAGGGCGAGGGCGAUGCCACCUACGGCAA |
| | GCUGACCCUGAAGUUCAUCUGCACCACCGGCAAGCUGCCCGUGC |
| | CCUGGCCCACCCUCGUGACCACCCUGACCUACGGCGUGCAGUGC |
| | UUCAGCCGCUACCCCGACCACAUGAAGCAGCACGACUUCUUCAA |
| | GUCCGCCAUGCCCGAAGGCUACGUCCAGGAGCGCACCAUCUUCU |
| | UCAAGGACGACGGCAACUACAAGACCCGCGCCGAGGUGAAGUUC |
| | GAGGGCGACACCCUGGUGAACCGCAUCGAGCUGAAGGGCAUCGA |
| | CUUCAAGGAGGACGGCAACAUCCUGGGGCACAAGCUGGAGUACA |
| | ACUACAACAGCCACAACGUCUAUAUCAUCACAUUUUACAGGCCA |
| | UG [symbol] AAAAGUUAUCAGGCAUGCACCUGGUAGCUAGUCU |
| | UUAAACCAAUAGAUUGCAUCGGUUUAAAAGGCAAGACCGUCA |
| | AAUUGCGGGAAAGGGGUCAACAGCCGUUCAGUACCAAGUCUC |
| | AGGGGAAACUUUGAGAUGGCCUUGCAAAGGGUAUGGUAAUAA |
| | GCUGACGGACAUGGUCCUAACCACGCAGCCAAGUCCUAAGUC |
| | AACAGAUCUUCUGUUGAUAUGGAUGCAGUUCACAGACUAAAU |
| | GUCGGUCGGGGAAGAUGUAUUCUUCUCAUAAGAUAUAGUCG [symbol] |
| | [symbol] CCUCUCCUUAAUGGGAGCUAGCGGAUGAAGUGAUGCAACAC |
| | UGGAGCCGCUGGGAACUAAUUACCAGUGGACAAUCGACGGAUA |
| | ACAGCAUAUCUAGAAAAAAAAAAAAAAAAAAAAAAAAAAAAAA |
| | AAAAAAAAAAAAAAAAAAAAAAAA |
| SEQ ID NO: 45 | CCGUCGAUUGUCCACUGGUCAAA [symbol] AAAAACAAAAAACA |
| [R1: Arm IV-Spacer 1- | AAAAAAACAAAAAAAAAACCAAAAAAACAAAACACAUUAAAAC |
| [symbol] - [symbol] | AGCCUGUGGGUUGUUCCCACCCGCAGGGCCCACUGGGCGCUAGC |
| GOI with a target site at the 3′ | ACACUGGUAUCCCGGUACCCUUGUGCGCCUGUUUUAUAUACCCU |
| end; Linker sequence: P1-ex-1- | CCCCCUUAUGUAACUUAGAAGUAUGAUUCAAACGGUCGACAGGC |
| Loop sequence-P1-ex-2; [symbol] ; | GGCUCAGUGCACCAACUGAGUCAUGACCAAGCACUUCUGUUACC |
| Anabaena ribozyme core: SEQ | CCGGACUGAGUAUCAAUAAGCUGUUCACACGGCUGAAGGAGAAA |
| ID NO: 48; | ACGUUCGUUACCCGGCCAAUUACUUCGAGAAACCUAGUACCACC |
| R2: [symbol] -Spacer 2- | AUGAAGGUUGCGCAGUGUUUCGCUCCACACAACCCCAGUGUAGA |
| Arm IV; Tail] | UCAGGUCGAUGAGUCACCGCAUUCCCCACGGGCGACCGUGGCGG |
| related to FIG. 49 | UGGCUGCGUUGGCGGCCUGCCCAUGGGGCAACCCAUGGGACGCU |
| | UCAAUACUGACAUGGUGUGAAGAGUCUAUUGAGCUAAUUGGUA |
| | GUCCUCCGGCCCCUGAAUGCGGCUAAUCCCAACUGUGGAGCAGA |
| | UACUCACAAACCAGUGAGCGGUCUGUCGUAACGGGCAACUCCGC |
| | AGCGGAACCGACUACUUUGGGUGUCCGUGUUUCUUUUUAUUCUU |
| | ACAUUGGCUGCUUAUGGUGACAAUUGACAAAUUGUUACCAUAU |
| | AGCUAUUGGAUUGGCCAUCCGGUGACAAACAGAGCUAUUGUUUA |
| | CUUGUUUGUUGGUUUCAUACCAUUAAAUUACAAGGUCUUAGAA |
| | ACUCUCAACUUUAUUUUGACACUCAAUACAGCAAAGCCACCAUG |
| | GAAGAUGCGAAGAACAUCAAGAAGGGACCUGCCCCGUUUUACCC |
| | UUUGGAGGACGGUACAGCAGGAGAACAGCUCCACAAGGCGAUGA |
| | AACGCUACGCCCUGGUCCCCGGAACGAUUGCGUUUACCGAUGCA |
| | CAUAUUGAGGUAGACAUCACAUACGCAGAAUACUUCGAAAUGUC |
| | GGUGAGGCUGGCGGAAGCGAUGAAGAGAUAUGGUCUUAACACU |
| | AAUCACCGCAUCGUGGUGUGUUCGGAGAACUCAUUGCAGUUUUU |
| | CAUGCCGGUCCUUGGAGCACUUUUCAUCGGGGUCGCAGUCGCGC |
| | CAGCGAACGACAUCUACAAUGAGCGGGAACUCUUGAAUAGCAUG |
| | GGAAUCUCCCAGCCGACGGUCGUGUUUGUCUCCAAAAAGGGGCU |
| | GCAGAAAAUCCUCAACGUGCAGAAGAAGCUCCCCAUUAUUCAAA |
| | AGAUCAUCAUUAUGGAUAGCAAGACAGAUUACCAAGGGUUCCAG |
| | UCGAUGUAUACCUUUGUGACAUCGCAUUUGCCGCCAGGGUUUAA |
| | CGAGUAUGACUUCGUCCCCGAGUCAUUUGACAGAGAUAAAACCA |
| | UCGCGCUGAUUAUGAACUCCUCGGGUAGCACCGGUUUGCCAAAG |
| | GGGGUGGCGUUGCCCCACCGCACUGCUUGUGUGCGGUUCUCGCA |
| | CGCUAGGGACCCUAUCUUUGGUAAUCAGAUCAUUCCCGACACAG |
| | CAAUCCUGUCCGUGGUACCUUUUCAUCACGGUUUUGGCAUGUUC |
| | ACGACUCUCGGCUAUUUGAUUUGCGGUUUCAGGGUCGUACUUAU |
| | GUAUCGGUUCGAGGAAGAGCUAUUUUUGAGAUCCUUGCAAGAU |
| | UACAAGAUCCAGUCGGCCCUCCUUGUGCCAACGCUUUUCUCAUU |
| | CUUUGCGAAAUCGACACUUAUUGAUAAGUAUGACCUUUCCAAUC |
| | UGCAUGAGAUUGCCUCAGGGGGAGCGCCGCUUAGCAAGGAAGUC |
| | GGGGAGGCAGUGGCCAAGCGCUUCCACCUUCCCGGAAUCCGGCA |
| | GGGAUACGGGCUCACGGAGACAACAUCCGCGAUCCUUAUCACGC |
| | CCGAGGGUGACGAUAAGCCGGGAGCCGUCGGAAAAGUGGUCCCC |
| | UUCUUUGAAGCCAAGGUCGUAGACCUCGACACGGGAAAAACCCU |
| | CGGAGUGAACCAGAGGGGCGAGCUCUGCGUGAGAGGGCCGAUGA |
| | UCAUGUCAGGUUACGUGAAUAACCCUGAAGCGACGAAUGCGCUG |
| | AUCGACAAGGAUGGGUGGUUGCAUUCGGGAGACAUUGCCUAUU |
| | GGGAUGAGGAUGAGCACUUCUUUAUCGUAGAUCGACUUAAGAG |
| | CUUGAUCAAAUACAAAGGCUAUCAGGUAGCGCCUGCCGAGCUCG |
| | AGUCAAUCCUGCUCCAGCACCCCAACAUUUUCGACGCCGGAGUG |
| | GCCGGGUUGCCCGAUGACGACGCGGGUGAGCUGCCAGCGGCCGU |
| | GGUAGUCCUCGAACAUGGGAAAACAAUGACCGAAAAGGAGAUCG |
| | UGGACUACGUAGCAUCACAAGUGACGACUGCGAAGAAACUGAGG |
| | GGAGGGGUAGUCUUUGUGGACGAGGUCCCGAAAGGCUUGACUG |
| | GGAAGCUUGACGCUCGCAAAAUCCGGGAAAUCCUGAUUAAGGCA |
| | AAGAAAGGCGGGAAAAUCGCUGUCUGAUAAAAAAAAACAAAAA |
| | AACAAAACAAACCUUAAAUAAUU [symbol] CCUUAAAGAAGAAAUUC |
| | UUUAAGUGGAUGCUCUCAAACUCAGGGAAACCUAAAUCUAGU |
| | UAUAGACAAGGCAAUCCUGAGCCAAGCCGAAGUAGUAAUUAG |
| | UAAGUCAACAAUAGAUGACUUACAACUAAUCGGAAGGUGCAG |
| | AGACUCGACGGGAGCUACCCUAACGUCAAGACGAGGGUAAAG |
| | AGAGAGUCCA [symbol] AAAGACCAGUGGACAAUCGACGGAUAA |
| | CAGCAUAUCUAGAAAAAAAAAAAAAAAAAAAAAAAAAAAAAAA |
| | AAAAAAAAAAAAAAAAAAAAAAA |
| SEQ ID NO: 46 | CCGUCGAUUGUCCACUGGUCGAUUAGUUU [symbol] CAUGGCCGACAA |
| [R1: Arm IV- [symbol] - | GCAGAAGAACGGCAUCAAGGCGAACUUCAAGAUCCGCCACAACA |
| [symbol] - [symbol] | UCGAGGACGGCAGCGUGCAGCUCGCCGACCACUACCAGCAGAAC |
| GOI with a target site at the 3′ | ACCCCCAUCGGCGACGGCCCCGUGCUGCUGCCCGACAACCACUAC |
| end; Linker sequence: Loop | CUGAGCACCCAGUCCGCCCUGAGCAAAGACCCCAACGAGAAGCG |
| sequence; [symbol] ; Tetrahymena | CGAUCACAUGGUCCUGCUGGAGUUCGUGACCGCCGCCGGGAUCA |
| ribozyme core: SEQ ID NO: 17; | CUCUCGGCAUGGACGAGCUGUACAAGUGAUAAACCGGUGCUGGA |
| R2: [symbol] - | GCCUCGGUGGCCAUGCUUCUUGCCCCUUGGGCCUCCCCCCAGCCC |
| 5′ half of P9.2-Arm IV; Tail] | CUCCUCCCCUUCCUGCACCCGUACCCCCGUGGUCUUUGAAUAAA |
| related to FIG. 52 | GUCUGAGUGGGCGGCCUCGAGAAAAAAAAAAAACAAAAAAAAA |
| | AAACCAAAAAAAAAAAAUAAAAAACUAAUUAAAACAGCGGAUG |
| | GGUACCCCACCAUCCGACCCACUGGGUGUAGUACUCUGGUACUU |
| | CGUACCUUUGUACGCCUGUUCUUCCCAUUGUACCCUUCCUGAAC |
| | UUCCAACCCAAGUAACGUUAGAAGCUCAACAUUUAGUACAACAG |
| | GAAGCACCACAUCCAGUGGUGUUUAGUACAAGCACUUCUGUUUC |
| | CCCGGAGCGAGGUAUAGGCUGUACCCACUGCCAAAAACCUUUAA |
| | CCGUUAUCCGCCAACCAACUACGUAAAAGCUAGUAGUAUUAUGU |
| | UUUUAACUAGGCGUUCGAUCAGGUGGAUUUCCCCUCCACUAGUU |
| | UGGUCGAUGAGGCUAGGAAUUCCCCACGGGUGACCGUGUCCUAG |
| | CCUGCGUGGCGGCCAACCCAGCUUAUGCUGGGACGCCUUUUUAU |
| | AGACAUGGUGUGAAGACUCGCAUGUGCUUGGUUGUGAUUCCUCC |
| | GGCCCCUGAAUGCGGCUAACCUUAACCCUGGAGCCUUGUGUCAC |
| | AAACCAGUGAUGAUAAGGUCGUAAUGAGCAAUUCCGGGACGGG |
| | ACCGACUACUUUGGGUGUCCGUGUUUCUUAUUUUUCUUAUUAUU |
| | GUCUUAUGGUCACAGCAUAUAUAUAACAUAUACUGUGAUCAUG |
| | GUGAGCAAGGGCGAGGAGCUGUUCACCGGGGUGGUGCCCAUCCU |
| | GGUCGAGCUGGACGGCGACGUAAACGGCCACAAGUUCAGCGUGU |
| | CUGGCGAGGGCGAGGGCGAUGCCACCUACGGCAAGCUGACCCUG |
| | AAGUUCAUCUGCACCACCGGCAAGCUGCCCGUGCCCUGGCCCAC |
| | CCUCGUGACCACCCUGACCUACGGCGUGCAGUGCUUCAGCCGCU |
| | ACCCCGACCACAUGAAGCAGCACGACUUCUUCAAGUCCGCCAUG |
| | CCCGAAGGCUACGUCCAGGAGCGCACCAUCUUCUUCAAGGACGA |
| | CGGCAACUACAAGACCCGCGCCGAGGUGAAGUUCGAGGGCGACA |
| | CCCUGGUGAACCGCAUCGAGCUGAAGGGCAUCGACUUCAAGGAG |
| | GACGGCAACAUCCUGGGGCACAAGCUGGAGUACAACUACAACAG |
| | CCACAACGUCUAUAUCACAUUUUACA [symbol] AAAAGUUAUCAG |
| | GCAUGCACCUGGUAGCUAGUCUUUAAACCAAUAGAUUGCAUC |
| | GGUUUAAAAGGCAAGACCGUCAAAUUGCGGGAAAGGGGUCAA |
| | CAGCCGUUCAGUACCAAGUCUCAGGGGAAACUUUGAGAUGGC |
| | CUUGCAAAGGGUAUGGUAAUAAGCUGACGGACAUGGUCCUAA |
| | CCACGCAGCCAAGUCCUAAGUCAACAGAUCUUCUGUUGAUAU |
| | GGAUGCAGUUCACAGACUAAAUGUCGGUCGGGGAAGAUGUAU |
| | UCUUCUCAUAAGAUAUAGUCG [symbol] GAACUAAUUACCAGUGGACA |
| | AUCGACGGAUAACAGCAUAUCUAGAAAAAAAAAAAAAAAAAAA |
| | AAAAAAAAAAAAAAAAAAAAAAAAAAAAAAAAAAA |
| SEQ ID NO: 47 | CCGUCGAUUGUCCACUGGUCGAUUAGUUU [symbol] CAUGGCCGACAA |
| [R1: Arm IV- [symbol] - | GCAGAAGAACGGCAUCAAGGCGAACUUCAAGAUCCGCCACAACA |
| [symbol] - [symbol] | UCGAGGACGGCAGCGUGCAGCUCGCCGACCACUACCAGCAGAAC |
| GOI with P10-1 at the 5′ end and | ACCCCCAUCGGCGACGGCCCCGUGCUGCUGCCCGACAACCACUAC |
| a target site at the 3′ end; Linker | CUGAGCACCCAGUCCGCCCUGAGCAAAGACCCCAACGAGAAGCG |
| sequence: P1-ex-1-Loop | CGAUCACAUGGUCCUGCUGGAGUUCGUGACCGCCGCCGGGAUCA |
| sequnence (including 5′ portion | CUCUCGGCAUGGACGAGCUGUACAAGUGAUAAACCGGUGCUGGA |
| of P10-2)-P1-ex-2 (3′ portion of | GCCUCGGUGGCCAUGCUUCUUGCCCCUUGGGCCUCCCCCCAGCCC |
| P10-2); [symbol] ; Tetrahymena | CUCCUCCCCUUCCUGCACCCGUACCCCCGUGGUCUUUGAAUAAA |
| ribozyme core: SEQ ID NO: 17; | GUCUGAGUGGGCGGCCUCGAGAAAAAAAAAAAACAAAAAAAAA |
| R2: [symbol] -5′ half of | AAACCAAAAAAAAAAAAUAAAAAACUAAUUAAAACAGCGGAUG |
| P9.2-Arm IV; Tail] | GGUACCCCACCAUCCGACCCACUGGGUGUAGUACUCUGGUACUU |
| related to FIG. 55 | CGUACCUUUGUACGCCUGUUCUUCCCAUUGUACCCUUCCUGAAC |
| | UUCCAACCCAAGUAACGUUAGAAGCUCAACAUUUAGUACAACAG |
| | GAAGCACCACAUCCAGUGGUGUUUAGUACAAGCACUUCUGUUUC |
| | CCCGGAGCGAGGUAUAGGCUGUACCCACUGCCAAAAACCUUUAA |
| | CCGUUAUCCGCCAACCAACUACGUAAAAGCUAGUAGUAUUAUGU |
| | UUUUAACUAGGCGUUCGAUCAGGUGGAUUUCCCCUCCACUAGUU |
| | UGGUCGAUGAGGCUAGGAAUUCCCCACGGGUGACCGUGUCCUAG |
| | CCUGCGUGGCGGCCAACCCAGCUUAUGCUGGGACGCCUUUUUAU |
| | AGACAUGGUGUGAAGACUCGCAUGUGCUUGGUUGUGAUUCCUCC |
| | GGCCCCUGAAUGCGGCUAACCUUAACCCUGGAGCCUUGUGUCAC |
| | AAACCAGUGAUGAUAAGGUCGUAAUGAGCAAUUCCGGGACGGG |
| | ACCGACUACUUUGGGUGUCCGUGUUUCUUAUUUUUCUUAUUAUU |
| | GUCUUAUGGUCACAGCAUAUAUAUAACAUAUACUGUGAUCAUG |
| | GUGAGCAAGGGCGAGGAGCUGUUCACCGGGGUGGUGCCCAUCCU |
| | GGUCGAGCUGGACGGCGACGUAAACGGCCACAAGUUCAGCGUGU |
| | CUGGCGAGGGCGAGGGCGAUGCCACCUACGGCAAGCUGACCCUG |
| | AAGUUCAUCUGCACCACCGGCAAGCUGCCCGUGCCCUGGCCCAC |
| | CCUCGUGACCACCCUGACCUACGGCGUGCAGUGCUUCAGCCGCU |
| | ACCCCGACCACAUGAAGCAGCACGACUUCUUCAAGUCCGCCAUG |
| | CCCGAAGGCUACGUCCAGGAGCGCACCAUCUUCUUCAAGGACGA |
| | CGGCAACUACAAGACCCGCGCCGAGGUGAAGUUCGAGGGCGACA |
| | CCCUGGUGAACCGCAUCGAGCUGAAGGGCAUCGACUUCAAGGAG |
| | GACGGCAACAUCCUGGGGCACAAGCUGGAGUACAACUACAACAG |
| | CCACAACGUCUAUAUCACAUUUUACAAUGGUAUAGAAAAGUUAU |
| | CAGGCAUGCACCUGGUAGCUAGUCUUUAAACCAAUAGAUUGC |
| | AUCGGUUUAAAAGGCAAGACCGUCAAAUUGCGGGAAAGGGGU |
| | CAACAGCCGUUCAGUACCAAGUCUCAGGGGAAACUUUGAGAU |
| | GGCCUUGCAAAGGGUAUGGUAAUAAGCUGACGGACAUGGUCC |
| | UAACCACGCAGCCAAGUCCUAAGUCAACAGAUCUUCUGUUGA |
| | UAUGGAUGCAGUUCACAGACUAAAUGUCGGUCGGGGAAGAUG |
| | UAUUCUUCUCAUAAGAUAUAGUCGGAGAACUAAUUACCAGUGG |
| | ACAAUCGACGGAUAACAGCAUAUCUAGAAAAAAAAAAAAAAAA |
| | AAAAAAAAAAAAAAAAAAAAAAAAAAAAAAAAAAAAAA |
| SEQ ID NO: 48 | CCUUAAAGAAGAAAUUCUUUAAGUGGAUGCUCUCAAACUCAGGG |
| (ribozyme core sequence derived | AAACCUAAAUCUAGUUAUAGACAAGGCAAUCCUGAGCCAAGCCG |
| from Anabaena sp. PCC7120 | AAGUAGUAAUUAGUAAGUCAACAAUAGAUGACUUACAACUAAU |
| group I intron) | CGGAAGGUGCAGAGACUCGACGGGAGCUACCCUAACGUCAAGAC |
| | GAGGGUAAAGAGAGAGUCCA |
| SEQ ID NO: 49 | AAAUAAUUGAGCCUUAAAGAAGAAAUUCUUUAAGUGGAUGCUC |
| (GenBank: AY768517.1, | UCAAACUCAGGGAAACCUAAAUCUAGUUAUAGACAAGGCAAUCC |
| fragment; Anabaena sp. | UGAGCCAAGCCGAAGUAGUAAUUAGUAAGUUAACAAUAGAUGA |
| PCC7120 group I intron; IGS, 5′ | CUUACAACUAAUCGGAAGGUGCAGAGACUCGACGGGAGCUACCC |
| half of P9.0 and [symbol] | UAACGUCAAGACGAGGGUAAAGAGAGAGUCCAAUUCUCAAAGC |
| [symbol] are marked) | CAAUAGGCAGUAGCGAAAGCUGCAA [symbol] |
| SEQ ID NO: 50 | TTAAAACAGCGGATGGGTACCCCACCATCCGACCCACTGGGTGTA |
| (5′ UTR comprising an IRES | GTACTCTGGTACTTCGTACCTTTGTACGCCTGTTCTTCCCATTGTAC |
| sequence from Human rhinovirus | CCTTCCTGAACTTCCAACCCAAGTAACGTTAGAAGCTCAACATTTA |
| B with a [symbol] according to | GTACAACAGGAAGCACCACATCCAGTGGTGTTTAGTACAAGCACT |
| some embodiments; ORF | TCTGTTTCCCCGGAGCGAGGTATAGGCTGTACCCACTGCCAAAAAC |
| encoding GFP with a [symbol] | CTTTAACCGTTATCCGCCAACCAACTACGTAAAAGCTAGTAGTATT |
| according to some other | ATGTTTTTAACTAGGCGTTCGATCAGGTGGATTTCCCCTCCACTAG |
| embodiments; 3′ UTR) | TTTGGTCGATGAGGCTAGGAATTCCCCACGGGTGACCGTGTCCTAG |
| | CCTGCGTGGCGGCCAACCCAGCTTATGCTGGGAC [symbol] TTATAG |
| | ACATGGTGTGAAGACTCGCATGTGCTTGGTTGTGATTCCTCCGGCC |
| | CCTGAATGCGGCTAACCTTAACCCTGGAGCCTTGTGTCACAAACCA |
| | GTGATGATAAGGTCGTAATGAGCAATTCCGGGACGGGACCGACTAC |
| | TTTGGGTGTCCGTGTTTCTTATTTTTCTTATTATTGTCTTATGGTCA |
| | CAGCATATATATAACATATACTGTGATCATGGTGAGCAAGGGCG |
| | AGGAGCTGTTCACCGGGGTGGTGCCCATCCTGGTCGAGCTGG |
| | ACGGCGACGTAAACGGCCACAAGTTCAGCGTGTCTGGCGAGG |
| | GCGAGGGCGATGCCACCTACGGCAAGCTGACCCTGAAGTTCA |
| | TCTGCACCACCGGCAAGCTGCCCGTGCCCTGGCCCACCCTCGT |
| | GACCACCCTGACCTACGGCGTGCAGTGCTTCAGCCGCTACCCC |
| | GACCACATGAAGCAGCACGACTTCTTCAAGTCCGCCATGCCCG |
| | AAGGCTACGTCCAGGAGCGCACCATCTTCTTCAAGGACGACGG |
| | CAACTACAAGACCCGCGCCGAGGTGAAGTTCGAGGGCGACAC |
| | CCTGGTGAACCGCATCGAGCTGAAGGGCATCGACTTCAAGGA |
| | GGACGGCAACATCCTGGGGCACAAGCTGGAGTACAACTACAA |
| | CAGCCACAACGT [symbol] CATGGCCGACAAGCAGAAGAACGGC |
| | ATCAAGGCGAACTTCAAGATCCGCCACAACATCGAGGACGGCA |
| | GCGTGCAGCTCGCCGACCACTACCAGCAGAACACCCCCATCG |
| | GCGACGGCCCCGTGCTGCTGCCCGACAACCACTACCTGAGCA |
| | CCCAGTCCGCCCTGAGCAAAGACCCCAACGAGAAGCGCGATC |
| | ACATGGTCCTGCTGGAGTTCGTGACCGCCGCCGGGATCACTCT |
| | CGGCATGGACGAGCTGTACAAGTGATAAACCGGTGCTGGAGCC |
| | TCGGTGGCCATGCTTCTTGCCCCTTGGGCCTCCCCCCAGCCCCTCCT |
| | CCCCTTCCTGCACCCGTACCCCCGTGGTCTTTGAATAAAGTCTGAG |
| | TGGGCGGCCTCGAGAAAAAAAAAAAACAAAAAAAAAAAACCAAA |
| | AAAAAAAAATAAAAAACTAA |
| SEQ ID NO: 51 | TTAAAACAGCCTGTGGGTTGTTCCCACCCGCAGGGCCCACTGGGC |
| (5′ UTR comprising an IRES | GCTAGCACACTGGTATCCCGGTACCCTTGTGCGCCTGTTTTATATA |
| sequence from Enterovirus B; | CCCTCCCCCTTATGTAACTTAGAAGTATGATTCAAACGGTCGACAG |
| ORF encoding luciferase; 3′ | GCGGCTCAGTGCACCAACTGAGTCATGACCAAGCACTTCTGTTACC |
| UTR comprising a [symbol] ) | CCGGACTGAGTATCAATAAGCTGTTCACACGGCTGAAGGAGAAAA |
| | CGTTCGTTACCCGGCCAATTACTTCGAGAAACCTAGTACCACCATG |
| | AAGGTTGCGCAGTGTTTCGCTCCACACAACCCCAGTGTAGATCAG |
| | GTCGATGAGTCACCGCATTCCCCACGGGCGACCGTGGCGGTGGCT |
| | GCGTTGGCGGCCTGCCCATGGGGCAACCCATGGGACGCTTCAATA |
| | CTGACATGGTGTGAAGAGTCTATTGAGCTAATTGGTAGTCCTCCGG |
| | CCCCTGAATGCGGCTAATCCCAACTGTGGAGCAGATACTCACAAA |
| | CCAGTGAGCGGTCTGTCGTAACGGGCAACTCCGCAGCGGAACCGA |
| | CTACTTTGGGTGTCCGTGTTTCTTTTTATTCTTACATTGGCTGCTTA |
| | TGGTGACAATTGACAAATTGTTACCATATAGCTATTGGATTGGCCA |
| | TCCGGTGACAAACAGAGCTATTGTTTACTTGTTTGTTGGTTTCATA |
| | CCATTAAATTACAAGGTCTTAGAAACTCTCAACTTTATTTTGACAC |
| | TCAATACAGCAAAGCCACCATGGAAGATGCGAAGAACATCAAG |
| | AAGGGACCTGCCCCGTTTTACCCTTTGGAGGACGGTACAGCAG |
| | GAGAACAGCTCCACAAGGCGATGAAACGCTACGCCCTGGTCC |
| | CCGGAACGATTGCGTTTACCGATGCACATATTGAGGTAGACAT |
| | CACATACGCAGAATACTTCGAAATGTCGGTGAGGCTGGCGGAA |
| | GCGATGAAGAGATATGGTCTTAACACTAATCACCGCATCGTGG |
| | TGTGTTCGGAGAACTCATTGCAGTTTTTCATGCCGGTCCTTGG |
| | AGCACTTTTCATCGGGGTCGCAGTCGCGCCAGCGAACGACATC |
| | TACAATGAGCGGGAACTCTTGAATAGCATGGGAATCTCCCAGC |
| | CGACGGTCGTGTTTGTCTCCAAAAAGGGGCTGCAGAAAATCCT |
| | CAACGTGCAGAAGAAGCTCCCCATTATTCAAAAGATCATCATT |
| | ATGGATAGCAAGACAGATTACCAAGGGTTCCAGTCGATGTATA |
| | CCTTTGTGACATCGCATTTGCCGCCAGGGTTTAACGAGTATGA |
| | CTTCGTCCCCGAGTCATTTGACAGAGATAAAACCATCGCGCTG |
| | ATTATGAACTCCTCGGGTAGCACCGGTTTGCCAAAGGGGGTGG |
| | CGTTGCCCCACCGCACTGCTTGTGTGCGGTTCTCGCACGCTAG |
| | GGACCCTATCTTTGGTAATCAGATCATTCCCGACACAGCAATC |
| | CTGTCCGTGGTACCTTTTCATCACGGTTTTGGCATGTTCACGA |
| | CTCTCGGCTATTTGATTTGCGGTTTCAGGGTCGTACTTATGTAT |
| | CGGTTCGAGGAAGAGCTATTTTTGAGATCCTTGCAAGATTACA |
| | AGATCCAGTCGGCCCTCCTTGTGCCAACGCTTTTCTCATTCTTT |
| | GCGAAATCGACACTTATTGATAAGTATGACCTTTCCAATCTGC |
| | ATGAGATTGCCTCAGGGGGAGCGCCGCTTAGCAAGGAAGTCG |
| | GGGAGGCAGTGGCCAAGCGCTTCCACCTTCCCGGAATCCGGC |
| | AGGGATACGGGCTCACGGAGACAACATCCGCGATCCTTATCAC |
| | GCCCGAGGGTGACGATAAGCCGGGAGCCGTCGGAAAAGTGGT |
| | CCCCTTCTTTGAAGCCAAGGTCGTAGACCTCGACACGGGAAAA |
| | ACCCTCGGAGTGAACCAGAGGGGCGAGCTCTGCGTGAGAGGG |
| | CCGATGATCATGTCAGGTTACGTGAATAACCCTGAAGCGACGA |
| | ATGCGCTGATCGACAAGGATGGGTGGTTGCATTCGGGAGACA |
| | TTGCCTATTGGGATGAGGATGAGCACTTCTTTATCGTAGATCG |
| | ACTTAAGAGCTTGATCAAATACAAAGGCTATCAGGTAGCGCCT |
| | GCCGAGCTCGAGTCAATCCTGCTCCAGCACCCCAACATTTTCG |
| | ACGCCGGAGTGGCCGGGTTGCCCGATGACGACGCGGGTGAGC |
| | TGCCAGCGGCCGTGGTAGTCCTCGAACATGGGAAAACAATGA |
| | CCGAAAAGGAGATCGTGGACTACGTAGCATCACAAGTGACGA |
| | CTGCGAAGAAACTGAGGGGAGGGGTAGTCTTTGTGGACGAGG |
| | TCCCGAAAGGCTTGACTGGGAAGCTTGACGCTCGCAAAATCCG |
| | GGAAATCCTGATTAAGGCAAAGAAAGGCGGGAAAATCGCTGT |
| | CTGATAAAAAAAAACAAAAAAACAAAACAAAC [symbol] AAAAACAAA |
| | AAACAAAAAAAACAAAAAAAAAACCAAAAAAACAAAACACA |
| SEQ ID NO: 52 | CCGUCGAUUGUCCACUGGUC |
| (5′ homology arm of Arm IV) | |
| SEQ ID NO: 53 | GACCAGUGGACAAUCGACGG |
| (3′ homology arm of Arm IV) | |
| SEQ ID NO: 54 | CCGUCGAUUGUCCACUGGUCAAAGAGAAUG |
| (R1 of SEQ ID NO: 45) | |
| SEQ ID NO: 55 | AUUCUCAAAGACCAGUGGACAAUCGACGG |
| (R2 of SEQ ID NO: 45) | |

1. An RNA construct comprising,
a first recognizer sequence (R1) comprising a first pairing sequence;
a nucleotide sequence of interest (GOI) comprising a target site at its 3′ end;
a ribozyme core sequence operably linked to an internal guide sequence (IGS), wherein the ribozyme core sequence encodes a ribozyme core having the catalytic activity of a group I intron ribozyme; and
a second recognizer sequence (R2) comprising a second pairing sequence substantially complementary to the first pairing sequence;
wherein
the 5′ end nucleotide of the IGS and the 3′ end nucleotide of the target site form a non-Watson-Crick base pair to define a 5′ splice site;
R1 and R2 are positioned at opposite ends of the RNA construct, such that hybridization of the first and second pairing sequences results in formation of a duplex-containing structure to define a 3′ splice site;
the GOI is positioned 5′ to the ribozyme core sequence and IGS; and
the RNA construct is capable of generating a circular RNA comprising the nucleotide sequence of interest through the catalytic activity of the ribozyme core.

2. The RNA construct according to claim 1 comprising, from 5′ end to 3′ end,
R1 comprising a first pairing sequence and a 3′ end nucleotide ‘N’ (ωN);
GOI comprising a target site at its 3′ end,
IGS;
Ribozyme core sequence; and
R2 comprising a second pairing sequence;
wherein
ωN is any naturally occurring or modified nucleotide; and
the first pairing sequence and the second pairing sequence are substantially complementary to form a duplex-containing structure upstream of the ωN to define the 3′ splice site.

3. The RNA construct according to claim 2, wherein ωN is guanine (ωG).

4. The RNA construct according to claim 1, wherein the ribozyme core sequence comprises a nucleotide sequence encoding the scaffold domain and catalytic domain of a group I intron; optionally wherein the ribozyme core sequence comprises or consists of the sequence from the IGS end to the sequence before the 5′ half of P9.0 duplex of a group I intron.

5. The RNA construct according to claim 1, wherein the ribozyme core sequence is derived from a group IC1 (e.g., from Tetrahymena sp. (e.g., T. thermophile, T. cosmopolitanis, T. hyperangularis, T. malaccensis or T. pigmentosa) or Pneumocystis sp. (e.g., Pneumocystis carinii), IC2, IC3 (e.g., from Anabaena sp. PCC7120 or Azoarcus sp. BH72) or IA2 (e.g., from Bacteriophage Twort) intron.

6. The RNA construct according to claim 1,
(A) wherein the ribozyme core sequence is derived from a Pneumocystis sp. group I intron; optionally wherein the Pneumocystis sp. group I intron comprises a nucleotide sequence selected from SEQ ID NOs: 32-36; optionally wherein the ribozyme core sequence comprises or consists of the nucleotide sequence of SEQ ID NO:19 or a nucleotide sequence having at least 95% sequence identity thereto;
(B) wherein the ribozyme core sequence is derived from a Tetrahymena sp. group I intron; optionally wherein the Tetrahymena thermophila group I intron comprises the nucleotide sequence of SEQ ID NO:12; optionally wherein the ribozyme core sequence comprises or consists of the nucleotide sequence of SEQ ID NO:17 or a nucleotide sequence having at least 95% sequence identity thereto; or
(C) wherein the ribozyme core sequence is derived from an Anabaena sp. group I intron; optionally wherein the ribozyme core sequence comprises or consists of the nucleotide sequence of SEQ ID NO:48 or a nucleotide sequence having at least 95% sequence identity thereto.

7. (canceled)

8. (canceled)

9. The RNA construct according to claim 1, wherein the duplex-containing structure comprises one or more base pairs.

10. The RNA construct according to claim 1, wherein the first pairing sequence comprises a nucleotide ‘N₁’ that is able to form a base pair with a nucleotide ‘n₁’ of the second pairing sequence, wherein ‘N₁’ is located at an ωN-i position in the RNA construct, and wherein i is an integer of 1-21; optionally wherein i is an integer of 1-11 or i is 1 or 2.

11. The RNA construct according to claim 10, wherein ‘N₁’ is the 3′ end nucleotide of a first contiguous sequence of 2-6 nucleotides in the first pairing sequence, ‘n₁’ is the 5′ end nucleotide of a second contiguous sequence in the second pairing sequence, wherein the first contiguous sequence is reverse complementary to the second contiguous sequence.

12. The RNA construct according to claim 1, wherein
the first and second pairing sequences each independently comprises 1-200 nucleotides;
optionally wherein the first pairing sequence comprises 2-20, 2-12, 4-10, 6, 7 or 8 nucleotides; and/or the second pairing sequence comprises 2-100, 5-80, 8-60, 10-50, 15, 20, 30, 40, 50, 60, 70, 80, 90 or 100; optionally wherein the second pairing sequence comprises 5-80 or 8-60 nucleotides.

13. The RNA construct according to claim 1, wherein
R1 further comprises a 5′ homology arm sequence located upstream of the first pairing sequence and R2 further comprises a 3′ homology arm sequence located downstream of the second pairing sequence, and the 5′ and 3′ homology arm sequences are substantially complementary.

14. An RNA construct comprising, from 5′ end to 3′ end,
a first recognizer sequence (R1) comprising a nucleotide sequence ‘(Nₓ)ₛ(N_{y})ₜ(ωN)’ at its 3′ end;
a nucleotide sequence of interest (GOI) comprising a target site at its 3′ end;
an internal guide sequence (IGS);
a ribozyme core sequence encoding a ribozyme core which has the catalytic activity of a group I intron ribozyme; and
a second recognizer sequence (R2) comprising a nucleotide sequence ‘(nₓ)_{w}’;
wherein
the 5′ end nucleotide of the IGS and the 3′ end nucleotide of the target site form a non-Watson-Crick base pair to define a 5′ splice site;
ωN, ‘Nₓ’, ‘nₓ’, and ‘N_{y}’ are each independently any naturally occurring or modified nucleotide;
t is an integer of 0-20;
s and w are each independently an integer of 1-200;
‘(Nₓ)ₛ’ and ‘(nₓ)_{w}’ are substantially complementary to form a duplex-containing structure upstream of the ωN to define a 3′ splice site; and
the RNA construct is capable of generating a circular RNA comprising the nucleotide sequence of interest through the catalytic activity of the ribozyme core.

15.-21. (canceled)

22. An RNA construct comprising, from 5′ end to 3′ end,
a first nucleotide sequence comprising a sequence from a nucleotide ‘N_{q}’ to the 3′ end of a group I intron,
a nucleotide sequence of interest (GOI) comprising a target site at its 3′ end,
an internal guide sequence (IGS), and
a second nucleotide sequence comprising a sequence from the IGS end to a nucleotide ‘Nₚ’ of a group I intron;
wherein
the 5′ end nucleotide of the IGS and the 3′ end nucleotide of the target site form a non-Watson-Crick base pair to define a 5′ splice site;
‘Nₚ’ and ‘N_{q}’ are independently selected from any nucleotide from the 3′ end nucleotide of the 5′ half to the 5′ end nucleotide of the 3′ half of P9.0 duplex of the group I intron, and
‘Nₚ’ is located upstream of ‘N_{q}’ in the group I intron.

23.-27. (canceled)

28. The RNA construct according to claim 1, wherein the non-Waton-Crick base pair formed between the 5′ end nucleotide of the IGS and the 3′ end nucleotide of the target site is
(a) guanine-uracil (G-u), wherein ‘G’ is the 5′ end nucleotide of the IGS and ‘u’ is the 3′ end nucleotide of the target site; or
(b) adenine-cytosine (A-c), wherein ‘A’ is the 5′ end nucleotide of the IGS and ‘c’ is the 3′ end nucleotide of the target site; or
(c) guanine-adenine (G-a), wherein ‘G’ is the 5′ end nucleotide of the IGS and ‘a’ is the 3′ end nucleotide of the target site.

29. The RNA construct according to claim 1, wherein the IGS and the target site form a P1 duplex mimic.

30. The RNA construct according to claim 1, wherein
the IGS has the structure of 5′-X(N)ₘ-3′,
the target site has the structure of 5′-(n)ₘx-3′,
‘X’ and ‘x’ are the nucleotides that form the non-Watson-Crick base pair,
each ‘N’ and ‘n’ is a nucleotide independently selected from A, G, C and U, and
m is an integer of 2-8, or m is an integer of 3-6, or m is an integer of 4-5;
optionally wherein 5′-(N)ₘ-3′ and 5′-(n)ₘ-3′ are reverse complementary.

31. The RNA construct according to claim 1, wherein
the IGS comprises a sequence ‘GNNNNN’ and the target site comprises a sequence ‘nnnnnu’; or
the IGS comprises a sequence ‘ANNNNN’ and the target site comprises a sequence ‘nnnnnc’;
wherein ‘NNNNN’ and ‘nnnnn’ are reverse complementary.

32. The RNA construct according to claim 1, wherein the RNA construct further comprises a linker sequence located between the target site and IGS.

33. The RNA construct according to claim 32, wherein
(A) the linker sequence comprises an unpaired sequence, and wherein the target site, the linker sequence and the IGS form a stem-loop structure;
(B) the linker sequence comprises, from 5′ end to 3′ end, a third pairing sequence, a loop sequence and a fourth pairing sequence, wherein the third and fourth pairing sequences form a P1 extension mimic; preferably, the P1 extension mimic comprises 1-3 reverse complementary base pairs; or
(C) the linker sequence comprises a fifth pairing sequence which can pair with a sixth pairing sequence in the 5′ region of the GOI to form a P10 duplex mimic; preferably, the P10 duplex mimic comprises 3-10 base pairs.

34.-37. (canceled)

38. The RNA construct according to claim 1, wherein the circular RNA does not contain an exogenous exon sequence.

39. A DNA construct comprising a sequence encoding the RNA construct according to claim 1.

40. A method of preparing a circular RNA comprising (i) providing a DNA construct according to claim 39 in a reaction solution, thereby allowing synthesis of the RNA construct by in vitro transcription of the DNA construct and allowing the RNA construct to self-splice, to produce a circular RNA, and (ii) recovering the circular RNA thus produced.


SEQUENCE LISTING (55 sequences)

SEQ ID NO: 1 - RNA, 2027 nt, synthetic construct
  source 1..2027: mol_type = other RNA
accgtcgattgtccactggtcgattagttttggagtactcgcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatcacattttacaggccatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaattaccagtggacaatcgacggataacagcatatctagaaaaaaaaaaaaaaaaaaaaaaaaaaaaaaaaaaaaaaaaaaaaaaaaaaaaaa

SEQ ID NO: 2 - RNA, 2027 nt, synthetic construct
  source 1..2027: mol_type = other RNA
accgtcgattgtccactggtcgattagttttggagtactcgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttacacattttacatctataagaaggcaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaattaccagtggacaatcgacggataacagcatatctagaaaaaaaaaaaaaaaaaaaaaaaaaaaaaaaaaaaaaaaaaaaaaaaaaaaaaa

SEQ ID NO: 3 - RNA, 1964 nt, synthetic construct
  source 1..1964: mol_type = other RNA
accgtcgattgtccactggtcgccttgacgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgccttttaccttctataagaaggcgaaagcggcgtgaaaacgttagctagtgatctggaataaattcagattgcgacactgtcaaattgcggggaagccctaaatattcaactactaagcagtttgtggaaacacagctgtggccgagttaatagccctgggtatagtaacaatgttgaatatgaatcttttgggagatgaaatgggtgatccgcagccaagtcctaagggcatttttgtctatggatgcagttcaacgactagatggcagtgggtattgtaaggaattgcagttttcttgcagtgcttaaggtatagtctgtcaaggcaccagtggacaatcgacggataacagcatatctagaaaaaaaaaaaaaaaaaaaaaaaaaaaaaaaaaaaaaaaaaaaaaaaaaaaaaaa

SEQ ID NO: 4 - RNA, 2045 nt, synthetic construct
  source 1..2045: mol_type = other RNA
accgtcgattgtccactggtcttacatctataagaaggcaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttacacattaccagtggacaatcgacggataacagcatatctagaaaaaaaaaaaaaaaaaaaaaaaaaaaaaaaaaaaaaaaaaaaaaaaaaaaaaa

SEQ ID NO: 5 - RNA, 1966 nt, synthetic construct
  source 1..1966: mol_type = other RNA
ggacgggcaagttacatctataagaaggcaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagttaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttacacattcttgcccgtcctctagaaaaaaaaaaaaaaaaaaaaaaaaaaaaaaaaaaaaaaaaaaaaaaaaaaaaaa

SEQ ID NO: 6 - RNA, 1963 nt, synthetic construct
  source 1..1963: mol_type = other RNA
accgtcgattgtccactggtcgccttggtgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgccttttaccttctataagaaggcgaaagcggcgtgaaaacgttagctagtgatctggaataaattcagattgcgacactgtcaaattgcggggaagccctaaatattcaactactaagcagtttgtggaaacacagctgtggccgagttaatagccctgggtatagtaacaatgttgaatatgaatcttttgggagatgaaatgggtgatccgcagccaagtcctaagggcatttttgtctatggatgcagttcaacgactagatggcagtgggtattgtaaggaattgcagttttcttgcagtgcttaaggtatagtctatcaaggcaccagtggacaatcgacggataacagcatatctagaaaaaaaaaaaaaaaaaaaaaaaaaaaaaaaaaaaaaaaaaaaaaaaaaaaaaa

SEQ ID NO: 7 - RNA, 2009 nt, synthetic construct
  source 1..2009: mol_type = other RNA
accgtcgattgtccactggtctggagtactcgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttacacattttacatctataagaaggcaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctggaccagtggacaatcgacggataacagcatatctagaaaaaaaaaaaaaaaaaaaaaaaaaaaaaaaaaaaaaaaaaaaaaaaaaaaaaa

SEQ ID NO: 8 - RNA, 2027 nt, synthetic construct
  source 1..2027: mol_type = other RNA
accgtcgattgtccactggtcgattagttttggagtactcgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgccttcttacacattttacatctataaaaaggcaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaattaccagtggacaatcgacggataacagcatatctagaaaaaaaaaaaaaaaaaaaaaaaaaaaaaaaaaaaaaaaaaaaaaaaaaaaaaa

SEQ ID NO: 9 - RNA, 1964 nt, synthetic construct
  source 1..1964: mol_type = other RNA
tggagtactcgcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatcacattttacaggccatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctggtaacagcatatctagaaaaaaaaaaaaaaaaaaaaaaaaaaaaaaaaaaaaaaaaaaaaaaaaaa

SEQ ID NO: 10 - RNA, 1966 nt, synthetic construct
  source 1..1966: mol_type = other RNA
accgtcgattgtccactggtcgattagttttcgcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatcacattttacaggccatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggagaactaattaccagtggacaatcgacggataacagcatatctagaaaaaaaaaaaaaaaaaaaaaaaaaaaaaaaaaaaaaaaaaaaaaaaaaaaaaa

SEQ ID NO: 11 - RNA, 1910 nt, synthetic construct
  source 1..1910: mol_type = other RNA
gtgttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgccttttaccttctataagaaggcgaaagcggcgtgaaaacgttagctagtgatctggaataaattcagattgcgacactgtcaaattgcggggaagccctaaatattcaactactaagcagtttgtggaaacacagctgtggccgagttaatagccctgggtatagtaacaatgttgaatatgaatcttttgggagatgaaatgggtgatccgcagccaagtcctaagggcatttttgtctatggatgcagttcaacgactagatggcagtgggtattgtaaggaattgcagttttcttgcagtgcttaaggtatagtctattaacagcatatctagaaaaaaaaaaaaaaaaaaaaaaaaaaaaaaaaaaaaaaaaaaaaaaaaaaaaaa

SEQ ID NO: 12 - RNA, 413 nt, Tetrahymena thermophila
  source 1..413: mol_type = rRNA
aaatagcaatatttacctttggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 13 - RNA, 21 nt, synthetic construct
  source 1..21: mol_type = other RNA
accgtcgattgtccactggtc

SEQ ID NO: 14 - RNA, 20 nt, synthetic construct
  source 1..20: mol_type = other RNA
accagtggacaatcgacgga

SEQ ID NO: 15 - RNA, 11 nt, synthetic construct
  source 1..11: mol_type = other RNA
ggacgggcaag

SEQ ID NO: 16 - RNA, 11 nt, synthetic construct
  source 1..11: mol_type = other RNA
cttgcccgtcc

SEQ ID NO: 17 - RNA, 285 nt, Tetrahymena thermophila
  source 1..285: mol_type = rRNA
aaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcg

SEQ ID NO: 18 - RNA, 387 nt, Tetrahymena thermophila
  source 1..387: mol_type = rRNA
aaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 19 - RNA, 297 nt, Pneumocystis carinii
  source 1..297: mol_type = rRNA
gaaagcggcgtgaaaacgttagctagtgatctggaataaattcagattgcgacactgtcaaattgcggggaagccctaaatattcaactactaagcagtttgtggaaacacagctgtggccgagttaatagccctgggtatagtaacaatgttgaatatgaatcttttgggagatgaaatgggtgatccgcagccaagtcctaagggcatttttgtctatggatgcagttcaacgactagatggcagtgggtattgtaaggaattgcagttttcttgcagtgcttaaggtatagtct

SEQ ID NO: 20 - RNA, 84 nt, synthetic construct
  source 1..84: mol_type = other RNA
gacctctccttaatgggagctagcggatgaagtgatgcaacactggagccgctgggaactaattaccagtggacaatcgacgga

SEQ ID NO: 21 - RNA, 41 nt, synthetic construct
  source 1..41: mol_type = other RNA
accgtcgattgtccactggtcgattagttttggagtactcg

SEQ ID NO: 22 - RNA, 75 nt, synthetic construct
  source 1..75: mol_type = other RNA
gacctctccttaatgggagctagcggatgaagtgatgcaacactggagccgctggaccagtggacaatcgacgga

SEQ ID NO: 23 - RNA, 32 nt, synthetic construct
  source 1..32: mol_type = other RNA
accgtcgattgtccactggtctggagtactcg

SEQ ID NO: 24 - RNA, 55 nt, synthetic construct
  source 1..55: mol_type = other RNA
gacctctccttaatgggagctagcggatgaagtgatgcaacactggagccgctgg

SEQ ID NO: 25 - RNA, 11 nt, synthetic construct
  source 1..11: mol_type = other RNA
tggagtactcg

SEQ ID NO: 26 - RNA, 31 nt, synthetic construct
  source 1..31: mol_type = other RNA
gagaactaattaccagtggacaatcgacgga

SEQ ID NO: 27 - RNA, 33 nt, synthetic construct
  source 1..33: mol_type = other RNA
accgtcgattgtccactggtcgattagttttcg

SEQ ID NO: 28 - RNA, 28 nt, synthetic construct
  source 1..28: mol_type = other RNA
gtcaaggcaccagtggacaatcgacgga

SEQ ID NO: 29 - RNA, 30 nt, synthetic construct
  source 1..30: mol_type = other RNA
accgtcgattgtccactggtcgccttgacg

SEQ ID NO: 30 - RNA, 28 nt, synthetic construct
  source 1..28: mol_type = other RNA
atcaaggcaccagtggacaatcgacgga

SEQ ID NO: 31 - RNA, 30 nt, synthetic construct
  source 1..30: mol_type = other RNA
accgtcgattgtccactggtcgccttggtg

SEQ ID NO: 32 - RNA, 344 nt, Pneumocystis carinii
  source 1..344: mol_type = rRNA
caccttctgagggtcatgaaagcggcgtgaaaacgttagctagtgatctggaataaattcagattgcgacactgtcaaattgcggggaagccctaaagattcaactactaagcagtttgtggaaacacagctgtggccgagttaatagccctgggtatagtaacaatgttgaatatgaatcttttgcgagatgaaatgggtgatccgcagccaagtcctaagggcatttttgtctatggatgcagttcaacgactagatggcagtgggtattgtaaggaattgcagttttcttgcagtgcttaaggtatagtctatcctctttcgaaagaaagagtatattgtg

SEQ ID NO: 33 - RNA, 361 nt, Pneumocystis carinii
  source 1..361: mol_type = rRNA
caaccttttgagggtcatgaaagcagcgtgaaaacgtttgctagtggtcaagttgggtattctgatttgactgcgacactgtcaaattgcggggaagccctaaagccttattcaccaagcaattgtggaaacactcttgtggccaggttaatagcctcgggtatggtaacagtagtaaggataaatgtgaaaaatgggttatccgcagccaaatcctaaggggaaaaagaattacaaatccgtattttcatcctatggatgcagttcaacgactagacggcagtgggtactgctcttttttactctgagtagtgcttaaggtatagtctgtccttttctgaaaagagagggaggggaggtg

SEQ ID NO: 34 - RNA, 342 nt, Pneumocystis wakefieldiae
  source 1..342: mol_type = rRNA
caaccttttgagggtcattaaagcggcgtgaaaacgttcgctagtgatctgaagccttttcaggttgcgacactgtcaaattgcggggaagccctaaagattcaactactaagcagcttgtggaaacacagctgtggccgagttaatagccctgggtatagtaacaatgttgaatatgaatcttgattgaagatgaaatgggtgatccgcagccaagtcctaaggacgtataatgtctatggatgcagttcaacgactagatggcagtgggtgttgttaagacttaggtttttacaatgcttaaggtatagtctattctctatcgaaagatagcgtatggtg

SEQ ID NO: 35 - RNA, 390 nt, Pneumocystis carinii
  source 1..390: mol_type = rRNA
tttttattggttcttctgcagtgcgccaaaggaagccttagcagcctgaaagggtgtatctccgcgactataaataaaaaggggattttaaatgctagtctgataaaaaaaggcgacattgccaaattgcgggaagtccctaaagattcaactactaagcagcttgtggaaacacagttgtggccgagttaatagccctgggtatagtaacaatgttgaatatgactcttaattgaggaaatgggtgatccgcagccaaatcctaaggacattttattgtctatggatgcagttcacaggccagatggcaatgggtatcctagtgggatatatatatatggatgcttaagatatggtcgagcttctctcgaaagagaggaggtagcactg

SEQ ID NO: 36 - RNA, 355 nt, Pneumocystis carinii
  source 1..355: mol_type = rRNA
caccttttgagggtcatgaaagcggcgcgaaagtgttagctagtgatccgaaaaataaattcgggttgcgacactgtcaaattgcggggagtccctaaagattcaactactaagcagcttgtggaaacacagttgtggccgagttaatagccctgggtatagtaacaatgttgaatatgactcttaattgaggaaatgggtgatccgcagccaaatcctaaggacattttattgtctatggatgcagttcagcgactagacggcagtgggtattgtagagatatggggttatttatggccttatctacaatgcttaaggtatagtctaatctctttcgaaagaaagagtagtgtg

SEQ ID NO: 37 - RNA, 27 nt, synthetic construct
  source 1..27: mol_type = other RNA
accgtcgattgtccactggtcgccttg

SEQ ID NO: 38 - RNA, 26 nt, synthetic construct
  source 1..26: mol_type = other RNA
caaggcaccagtggacaatcgacgga

SEQ ID NO: 39 - RNA, 1907 nt, synthetic construct
  source 1..1907: mol_type = other RNA
tcgcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatcacattttacaggccatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggataacagcatatctagaaaaaaaaaaaaaaaaaaaaaaaaaaaaaaaaaaaaaaaaaaaaaaaaaaaaaa

SEQ ID NO: 40 - RNA, 1919 nt, synthetic construct
  source 1..1919: mol_type = other RNA
aacaccaagcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatcacattttacaggccatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcgaaccacaataacagcatatctagaaaaaaaaaaaaaaaaaaaaaaaaaaaaaaaaaaaaaaaaaaaaaaaaaaaaaa

SEQ ID NO: 41 - RNA, 1978 nt, synthetic construct
  source 1..1978: mol_type = other RNA
accgtcgattgtccactggtcgattagtttaacaccaagcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatcacattttacaggccatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcgaaccacaagaactaattaccagtggacaatcgacggataacagcatatctagaaaaaaaaaaaaaaaaaaaaaaaaaaaaaaaaaaaaaaaaaaaaaaaaaaaaaa

SEQ ID NO: 42 - RNA, 39 nt, synthetic construct
  source 1..39: mol_type = other RNA
accgtcgattgtccactggtcgattagtttaacaccaag

SEQ ID NO: 43 - RNA, 37 nt, synthetic construct
  source 1..37: mol_type = other RNA
aaccacaagaactaattaccagtggacaatcgacgga

SEQ ID NO: 44 - RNA, 2027 nt, synthetic construct
  source 1..2027: mol_type = other RNA
accgtcgattgtccactggtcgattagttttggagtacgagcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatcacattttacaggccatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcgtccctctccttaatgggagctagcggatgaagtgatgcaacactggagccgctgggaactaattaccagtggacaatcgacggataacagcatatctagaaaaaaaaaaaaaaaaaaaaaaaaaaaaaaaaaaaaaaaaaaaaaaaaaaaaaa

SEQ ID NO: 45 - RNA, 2818 nt, synthetic construct
  source 1..2818: mol_type = other RNA
ccgtcgattgtccactggtcaaagagaatgaaaaacaaaaaacaaaaaaaacaaaaaaaaaaccaaaaaaacaaaacacattaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagccaccatggaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaactcctcgggtagcaccggtttgccaaagggggtggcgttgccccaccgcactgcttgtgtgcggttctcgcacgctagggaccctatctttggtaatcagatcattcccgacacagcaatcctgtccgtggtaccttttcatcacggttttggcatgttcacgactctcggctatttgatttgcggtttcagggtcgtacttatgtatcggttcgaggaagagctatttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaatccggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataaccctgaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtctgataaaaaaaaacaaaaaaacaaaacaaaccttaaataattgagccttaaagaagaaattctttaagtggatgctctcaaactcagggaaacctaaatctagttatagacaaggcaatcctgagccaagccgaagtagtaattagtaagtcaacaatagatgacttacaactaatcggaaggtgcagagactcgacgggagctaccctaacgtcaagacgagggtaaagagagagtccaattctcaaagaccagtggacaatcgacggataacagcatatctagaaaaaaaaaaaaaaaaaaaaaaaaaaaaaaaaaaaaaaaaaaaaaaaaaaaaaa

SEQ ID NO: 46 - RNA, 1955 nt, synthetic construct
  source 1..1955: mol_type = other RNA
ccgtcgattgtccactggtcgattagttttcgcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacattttacagtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggagaactaattaccagtggacaatcgacggataacagcatatctagaaaaaaaaaaaaaaaaaaaaaaaaaaaaaaaaaaaaaaaaaaaaaaaaaaaaaa

SEQ ID NO: 47 - RNA, 1958 nt, synthetic construct
  source 1..1958: mol_type = other RNA
ccgtcgattgtccactggtcgattagttttcgcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaattaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacattttacaatggtatagaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggagaactaattaccagtggacaatcgacggataacagcatatctagaaaaaaaaaaaaaaaaaaaaaaaaaaaaaaaaaaaaaaaaaaaaaaaaaaaaaa

SEQ ID NO: 48 - RNA, 195 nt, Anabaena sp.
  source 1..195: mol_type = tRNA
ccttaaagaagaaattctttaagtggatgctctcaaactcagggaaacctaaatctagttatagacaaggcaatcctgagccaagccgaagtagtaattagtaagtcaacaatagatgacttacaactaatcggaaggtgcagagactcgacgggagctaccctaacgtcaagacgagggtaaagagagagtcca

SEQ ID NO: 49 - RNA, 249 nt, Anabaena sp.
  source 1..249: mol_type = tRNA
aaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 50 - DNA, 1521 bp, synthetic construct
  source 1..1521: mol_type = other DNA
ttaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcttatgctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccggtgctggagcctcggtggccatgcttcttgccccttgggcctccccccagcccctcctccccttcctgcacccgtacccccgtggtctttgaataaagtctgagtgggcggcctcgagaaaaaaaaaaaacaaaaaaaaaaaaccaaaaaaaaaaaataaaaaactaa

SEQ ID NO: 51 - DNA, 2483 bp, synthetic construct
  source 1..2483: mol_type = other DNA
ttaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagccaccatggaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaactcctcgggtagcaccggtttgccaaagggggtggcgttgccccaccgcactgcttgtgtgcggttctcgcacgctagggaccctatctttggtaatcagatcattcccgacacagcaatcctgtccgtggtaccttttcatcacggttttggcatgttcacgactctcggctatttgatttgcggtttcagggtcgtacttatgtatcggttcgaggaagagctatttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaatccggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataaccctgaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtctgataaaaaaaaacaaaaaaacaaaacaaaccttaaaaacaaaaaacaaaaaaaacaaaaaaaaaaccaaaaaaacaaaacaca

SEQ ID NO: 52 - RNA, 20 nt, synthetic construct
  source 1..20: mol_type = other RNA
ccgtcgattgtccactggtc

SEQ ID NO: 53 - RNA, 20 nt, synthetic construct
  source 1..20: mol_type = other RNA
gaccagtggacaatcgacgg

SEQ ID NO: 54 - RNA, 30 nt, synthetic construct
  source 1..30: mol_type = other RNA
ccgtcgattgtccactggtcaaagagaatg

SEQ ID NO: 55 - RNA, 29 nt, synthetic construct
  source 1..29: mol_type = other RNA
attctcaaagaccagtggacaatcgacgg